(19) United States
(12) Patent Application Publication
(10) Pub. No.: US 2016/0287717 A1
(43) Pub. Date: Oct. 6, 2016

(54) Core and Surface Modification of Mesoporous Silica Nanoparticles to Achieve Cell Specific Targeting In Vivo.

(71) Applicants: STC.UNM, Albuquerque, NM (US); SANDIA CORPORATION, Albuquerque, NM (US)

(72) Inventors: Charles Jeffrey Brinker, Albuquerque, NM (US); Jason TOWNSON, Albuquerque, NM (US); Yu-Shen LIN, Albuquerque, NM (US); Paul N. DURFEE, Rio Rancho, NM (US)

(21) Appl. No.: 15/023,093

(22) PCT Filed: Sep. 18, 2014
(86) PCT No.: PCT/US2014/056312

§ 371 Date: Mar. 18, 2016

Related U.S. Application Data
(60) Provisional application No. 61/879,524, filed on Sep. 18, 2013.
(60) Provisional application No. 61/879,512, filed on Sep. 18, 2013.

(51) Int. Cl. A61K 47/48; A61K 9/51

(52) U.S. Cl. CPC A61K 47/48215; A61K 9/5146; A61K 9/5115; A61K 47/48861; A61K 47/48884; A61K 47/48192

(57) ABSTRACT

In one aspect, the invention provides mesoporous silica nanoparticles (MSNPs), monodisperse populations of MSNPs and related protocells which exhibit single cell binding specificity to the substantial exclusion of non-targeted cells. For example, MSNPs and protocells of the invention may be used to target specific delivery of therapeutic agents to cancer cells or to specific blood vessel types (e.g. in the arterial, venous and/or capillary vessels or any combination of vessels). Related protocells, pharmaceutical compositions and therapeutic and diagnostic methods are also provided.

### CROSS-REFERENCE TO RELATED APPLICATIONS

This application claims the benefit of priority from U.S. Provisional Patent Application Ser. No. 61/879,512, filed Sep. 18, 2013, and entitled “Core and Surface Modification of Mesoporous Silica Nanoparticles to Achieve Cell Specific Targeting In Vivo”, and U.S. Provisional Patent Application Ser. No. 61/879,524, filed Sep. 18, 2013, and entitled “Core and Surface Modification of Mesoporous Silica Nanoparticles to Achieve Vascular Type Specific Arrest (i.e., in the Arteriol, Venous or Capillary Bed) For Delivery of Cargo Including Imaging and/or Therapeutic Cargo to Endothelial Cells In Vivo”. These provisional applications and their complete contents are hereby incorporated by reference in their entirety.

### STATEMENT REGARDING FEDERAL FUNDING

This invention was made with government support under DE-AC04-94AL85000 awarded by the U.S. Department of Energy and U01 CA15179201 by the National Institutes of Health. The government has certain rights in the invention.

### FIELD OF THE INVENTION

In one aspect, the invention provides mesoporous silica nanoparticles (MSNPs) and related protocells which exhibit single cell binding specificity to the substantial exclusion of non-targeted cells. For example, MSNPs and protocells of the invention may be used to target specific delivery of therapeutic agents to cancer cells or to specific blood vessel types (e.g. in the arterial, venous and/or capillary vessels or any combination of vessels). Related protocells, pharmaceutical compositions and therapeutic and diagnostic methods are also provided. Pharmaceutical compositions comprising MSNPs and protocells adapted for administration via intravenous, intramuscular, intraperitoneal, retro-orbital and subcutaneous injection routes and methods of administration, treatment and diagnostics utilizing these compositions are additional aspects of the present invention.

### BACKGROUND OF THE INVENTION

Nanoparticle (NP)/cell interactions, particularly in complex in vivo microenvironments, are regulated by an intricate spatiotemporal interplay of numerous biological and NP characteristics. Multiple NP physicochemical properties including, at the most basic level, material composition, size, shape, surface charge, and surface chemistry, have all been reported to play significant roles.¹⁻³ However, the relative importance of these diverse NP physicochemical properties in regulating interactions with various biological systems remains incompletely understood.¹ As such, achieving or avoiding cell-type specific interactions in vivo requires an improved understanding of the relative roles of these diverse NP properties, as well an ability to exert a high level of control over these properties during NP synthesis.

While the existing paradigm dictates that decreased size, neutral or negative zeta (ζ) potential, and extent of PEGylation are correlated with increased circulation time (i.e., reduced interaction with host cells),⁴ the manner in which these combined physicochemical properties conspire to direct in vivo cellular interactions has not been elucidated through careful systematic studies, and the nature of these interactions is likely to vary significantly by particle formulation and cell type. As amination of particles is commonly used in various particle modification schemes to enable labeling or targeting, enhance binding and internalization,⁵ etc., here we attempt to further elucidate how the exposure of surface amines affects interaction of PEGylated, colloidally stable MSNP with diverse cell types both in vitro and in vivo.

### SUMMARY OF THE INVENTION

In vitro and in vivo behavior of the MSNPs described and claimed herein illustrate the relative importance of charged molecule exposure and spatial arrangement versus zeta (ζ) potential and/or particle size as determinants of nonspecific binding and biodistribution. We have discovered that uniform spatial distribution of charge presented within a PEG or PEG-like background for quaternary amines (e.g. PEG-NMe3+) confers both colloidal stability and protein corona neutrality, which in turn correlate with minimal nonspecific binding in vivo and prolonged circulation (and potentially opsonization neutrality), as evidenced by DLS. Such NP characteristics are expected to be ideal for maximizing the enhanced permeability and retention (EPR) effect or for binding and delivery to targeted circulating cells. In contrast, charge-matched PEG-PEI particles/interactions (e.g., amines having primary amines) displaying surface-exposed, branched amines, although colloidally stable, immediately form a protein corona and exhibit rapid nonspecific binding to endothelial and WBCs and arrest within the CAM. These characteristics are of potential interest for in vivo WBC and vascular labeling.

In one embodiment, the invention provides a population of optionally (preferably) PEGylated, monodisperse mesoporous silica nanoparticles (MSNPs) that are aminated with a composition comprising a primary amine group and that exhibit a non-uniform surface charge distribution and colloidal stability, wherein the MSNPs have a diameter ranging from about 25 nm to about 300 nm (more preferably, depending on route of administration of less than 50 about nm, even more preferably, less than about 30 nm), a pore size of between about 1 nm to about 200 nm, a surface area of between about 100-1,000 m²/g, and a Zeta potential (ζ) of between about −40 mV to about +40 mV (often greater than 0 mV to promote non-specific binding) and wherein upon administration in vivo, the MSNPs exhibit non-specific binding to white blood cells and arterial, venous and/or capillary vessels or combinations thereof. Core and surface modification of these MSNPs achieve vascular type specific arrest (i.e., in the arterial, venous or capillary bed) for delivery in vivo of cargo including imaging agents (e.g. rhodamine B isothiocynate) and/or therapeutic agents to endothelial cells.

“A composition comprising a primary amine group” or “primary amine-containing silane” is used to describe a silane compound containing a primary amine group which can be incorporated into MSNPs during production/formation and such compositions include, but are not limited to, a composition selected from the group consisting of trimethoxy-silylpropyl-modified polyethyleneimine (MW=1500-1800, PEI-silane), (3-aminopropyl)triethoxysilane, (3-Aminopropyl)trimethoxysilane, 3-Aminopropylmethyldiethoxysilane, 3-Aminopropyldimethylethoxysilane and mixtures thereof. In general, the amount of the primary amine containing silanes which are used to produce MSNPs in certain embodiments according to the present invention represent about 0.05% to about 25% (about 0.1% to about 20%, about 0.5% to about 15%, about 1% to about 10%, about 2.5% to about 7.5%, about 0.25% to about 5%, about 0.75% to about 15%) by weight of these monomers in combination with the silane monomers which are typically used to form MSNPs, which monomers will optionally (preferably) include PEG-containing silane monomers as otherwise described herein.

In another embodiment, the invention provides a population of optionally (preferably) PEGylated, monodisperse mesoporous silica nanoparticles (MSNPs) that are aminated with a composition that does not comprise a primary amine group (preferably, a quaternary amine, but such compositions may also include a tertiary amine and/or a secondary amine, depending on the desired zeta potential and the amount of non-specific binding to endothelial cells desired) and that exhibit a uniform surface charge distribution and colloidal stability, wherein the MSNPs have a diameter ranging from about 25 nm to about 300 nm (more preferably, less than 50 nm, even more preferably, less than 30 nm, depending upon route of administration), a pore size of between about 1 nm to about 200 nm, a surface area of between about 100-1,000 m²/g, and a Zeta potential (ζ) of between about −40 mV to about +40 mV (more preferably less than 0 mV in order to lessen/minimize non-specific binding to endothethilial cells/tissue and enhance distribution and residence times over a larger number of tissues and areas) and wherein upon administration in vivo, the MSNPs exhibit minimal non-specific binding and prolonged circulation. Core and surface modification of these MSNPs enable in vivo targeting of cargo including imaging agents (e.g. rhodamine B isothiocynate) and/or therapeutic agents to targets preferably including (1) a cancer cell (2) kidney tissue (3) lung tissue (4) pancreatic tissue (5) a bacterium, or (6) a virus.

“A composition that does not comprise a primary amine group” includes, but is not limited to, a composition containing a quaternary amine selected from the group consisting of N-trimethoxysilylpropyl-N,N,N-trimethyl ammonium chloride (TMAC-silane, MW 258), and related silyl compounds which contain a quaternary amine group (a quaternary amine-modified silane compound). These compounds contain a quaternary amine group and a silyl group which can be used as silyl-containing monomers (in conjunction with other silyl monomers) to form MSNPs hereunder. These compounds are preferably quaternary amine containing groups because they provide a uniform charge surface, especially in conjunction with PEG or PEG-like groups (often zwitterionic silyl groups), but may also include a compound containing a tertiary amine and/or a secondary amine (e.g. a tertiary amine modified silane or a secondary amine modified silane). Exemplary tertiary amine-modified silanes for use in the present invention include N¹-(3-Trimethoxylsilylpropyl)diethylenetriamine, among others, including 3-(trimethoxysilyl)propyl-di-n-octylmethyl-ammonium chloride; 3-(trimethoxysilyl)propyl-n-octyldimethyl-ammonium chloride; 3-(trimethoxysilyl)propyl-di-n-nonylmethyl-ammonium chloride; 3-(trimethoxysilyl)propyl-n-nonyldimethyl-ammonium chloride; 3-(trimethoxysilyl)propyl-di-decylmethyl-ammonium chloride; 3-(trimethoxysilyl)propyl-di-n-undecylmethyl-ammonium chloride; 3-(trimethoxysilyl)propyl-n-undecyldimethyl-ammonium chloride; 3-(trimethoxysilyl)propyl-di-n-dodecylmethyl-ammonium chloride; 3-(trimethoxysilyl)propyl-n-dodecyldimethyl-ammonium chloride; 3-(trimethoxysilyl)propyl-di-n-tridecyldimethyl-ammonium chloride; 3-(trimethoxysilyl)propyl-n-tridecyldimethyl-ammonium chloride; 3-(trimethoxysilyl)propyl-di-n-tetradecylmethyl-ammonium chloride; 3-(trimethoxysilyl)propyl-n-tetradecyldimethyl-ammonium chloride; 3-(triethoxysilyl)propyl-di-n-octylmethyl-ammonium chloride; 3-(triethoxysilyl)propyl-n-octyldimethyl-ammonium chloride; 3-(triethoxysilyl)propyl-di-n-nonylmethyl-ammonium chloride; 3(triethoxysilyl)propyl-n-nonyldimethyl-ammonium chloride; 3-(triethoxysilyl)propyl-di-n-decylmethyl-ammonium chloride; 3-(triethoxysilyl)propyl-n-decyldimethyl-ammonium chloride; 3-(triethoxysilyl)propyl-di-n-undecylmethyl-ammonium chloride; 3-(triethoxysilyl)propyl-n-undecyldimethyl-ammonium chloride; 3-(triethoxysilyl)propyl-di-n-dodecylmethyl-ammonium chloride; 3-(triethoxysilyl)propyl-n-dodecyldimethyl-ammonium chloride; 3-(triethoxysilyl)propyl-di-n-tridecylmethyl-ammonium chloride; 3-(triethoxysilyl)propyl-n-tridecyldimethyl-ammonium chloride; 3-(triethoxysilyl)propyl-di-n-tetradecylmethyl-ammonium chloride; 3-(triethoxysilyl)propyl-n-tetradecyldimethyl-ammonium chloride; 3-(tripropoxysilyl)propyl-di-n-octylmethyl-ammonium chloride; 3-(tripropoxysilyl)propyl-n-octyldimethyl-ammonium chloride; 3-(tripropoxysilyl)propyl-di-n-nonylmethyl-ammonium chloride; 3-(tripropoxysilyl)propyl-n-nonyldimethyl-ammonium chloride; 3-(tripropoxysilyl)propyl-di-n-decylmethyl-ammonium chloride; 3-(tripropoxysilyl)propyl-n-decyldimethyl-ammonium chloride; 3-(tripropoxysilyl)propyl-di-n-undecylmethyl-ammonium chloride; 3-(tripropoxysilyl)propyl-n-undecyldimethyl-ammonium chloride; 3-(tripropoxysilyl)propyl-di-n-dodecylmethyl-ammonium chloride; 3-(tripropoxysilyl)propyl-n-dodecyldimethyl-ammonium chloride; 3-(tripropoxysilyl)propyl-di-n-tridecylmethyl-ammonium chloride; 3-(tripropoxysilyl)propyl-n-tridecyldimethyl-ammonium chloride; 3-(tripropoxysilyl)propyl-di-n-tetradecylmethyl-ammonium chloride; 3-(tripropoxysilyl)propyl-n-tetradecyldimethyl-ammonium chloride; 3-(tributoxysilyl)propyl-di-n-octylmethyl-ammonium chloride; 3-(tributoxysilyl)propyl-n-octyldimethyl-ammonium chloride; 3-(tributoxysilyl)propyl-di-n-nonylmethyl-ammonium chloride; 3-(tributoxysilyl)propyl-n-nonyldimethyl-ammonium chloride; 3-(tributoxysilyl)propyl-di-n-decylmethyl-ammonium chloride; 3-(tributoxysilyl)propyl-n-decyldimethyl-ammonium chloride; 3-(tributoxysilyl)propyl-di-n-undecylmethyl-ammonium chloride; 3-(tributoxysilyl)propyl-n-undecyldimethyl-ammonium chloride; 3-(tributoxysilyl)propyl-di-n-dodecylmethyl-ammonium chloride; 3-(tributoxysilyl)propyl-n-dodecyldimethyl-ammonium chloride; 3-(tributoxysilyl)propyl-di-n-tridecylmethyl-ammonium chloride; 3-(tributoxysilyl)propyl-n-tridecyldimethyl-ammonium chloride; 3-(tributoxysilyl)propyl-di-n-tetradecylmethyl-ammonium chloride; 3-(tributoxysilyl)propyl-n-tetradecyldimethyl-ammonium chloride and mixtures thereof. Exemplary secondary amine-modified silanes (e.g., diamines which may contain both primary and secondary amines) for use in the present invention include N-[3-(Trimethoxysilyl)propyl]ethylenediamine, N-2-(Aminoethyl)-3-aminopropylmethyldimethoxysilane, N-(2-Aminoethyl)-3-aminoisobutyldimethylmethoxysilane, N-(6-Aminohexyl)aminopropyltrimethoxysilance and mixtures thereof. In general, the amount of the quaternary amine-containing silanes which optionally are used to produce MSNPs according to the present invention represent about about 0.05% to about 25% (about 0.1% to about 20%, about 0.5% to about 15%, about 1% to about 10%, about 2.5% to about 7.5%, about 0.25% to about 5%, about 0.75% to about 15%) by weight of these monomers in combination with the silane monomers which are typically used to form MSNPs. Of course secondary and tertiary amine-containing silanes may also be used within the same general weight range to effect binding non-specific binding characteristics which fall somewhere between the primary amines (high non-specific binding) and the quaternary amines (very low non-specific binding).

The size, charge, charge exposure and PEGylation of the MSNPs and protocells described herein can be controlled such that specifically tuned particles can be controllably deposited within certain tissue types (e.g. in the arterial, venous and/or capillary vessels or any combination of vessels). To enhance binding specificity, the MSNPs may be combined with targeting peptide or ligand. We have determined that an increasing cationic charge results in more localized binding to vasculature, whereas a less cationic, neutral or anionic charge results in broader in vivo dispersal.

The invention includes protocells in which the novel MSNPs described herein are encapsulated within a lipid bi- or multilayer.

By modifying MSNPs core (size, shape, mass) and surface properties, we can alter in vivo biodistribution by changing the proportion of particles arrested in different types of vasculature (e.g. capillary versus arterial or venous system). This control over the particles allows for physiochemical targeting of specific vasculature (and thereby tissues) and can be further modified to incorporate single cell type specific binding in the vasculature. (The term “binding” as used herein includes MSNPs and/or protocell binding to bacteria in vivo.)

The in vitro and in vivo behavior of the MSNPs described and claimed herein illustrate the relative importance of charged molecule exposure and spatial arrangement versus Zeta (ζ) potential and/or particle size as determinants of nonspecific binding and biodistribution. As described further in Example 3 herein, we have discovered that uniform spatial distribution of charge presented within a PEG background for PEG-NMe3+ confers both colloidal stability and protein corona neutrality, which in turn correlate with minimal nonspecific binding in vivo and prolonged circulation (and potentially opsonization neutrality), as evidenced by DLS. Such NP characteristics are expected to be ideal for maximizing the enhanced permeability and retention (EPR) effect or for binding and delivery to targeted, including circulating cells. In contrast, charge-matched PEG-PEI particles displaying surface-exposed, primary amines, including branched amines, although colloidally stable, immediately form a protein corona and exhibit rapid nonspecific binding to endothelial and WBCs and arrest within the CAM. These characteristics are of potential interest for in vivo WBC and vascular labeling.

In still other embodiments, the invention includes methods of treatment and diagnostic methods which use the novel MSNPs and protocells described herein to treat and/or diagnose a variety of disorders, including cancers, bacterial and viral infections, vascular disorders and inflammatory diseases and disorders as otherwise described herein.

The present invention also relates to the discovery that MSNPs and protocells which are monodisperse and less than 50 nm in average diameter, often 30 nm or less in diameter (in many instances MSNPs and protocells which are less than 25 nm in diameter may be preferred, especially for subcutaneous administration) can be used to effectively deliver cargo therefrom (especially therapeutic agents) after administration to a patient or subject by intravenous, intramuscular, intraperitoneal, retro-orbital and subcutaneous injection routes. In a particular embodiment, compositions according to the present invention which are administered pursuant to the present invention, and in particular subcutaneously which have not been modified with an amine or if modified, modified with a quaternary amine pursuant to the present invention, are shown to have excellent biodistribution after administration, in contrast to compositions wherein the protocells are larger in diameter (e.g. above about 30-50 nm in diameter) and which contain primary, and to a lesser extent, secondary and tertiary amines. Accordingly, the present invention may be used effectively for administering agents which have not been traditionally administered to patients for therapeutic and or diagnostic purposes by intravenous, intramuscular, intraperitoneal, retro-orbital and subcutaneous injection routes in a much more efficient manner than prior art methods and additionally, the present compositions and methods may be formulated for numerous therapeutic agents, including drugs, nucleic acids and polypeptides, among others and/or diagnostic agents which have exhibited poor biodistribution/bioavailability before the advent of the present invention.

These and other aspects of the invention are described further in the Detailed Description of the Invention.

### BRIEF DESCRIPTION OF THE FIGURES

FIG. 1 shows the importance of colloidal stability for nanotherapeutics made pursuant to the present invention. As determined in the experiment(s) of Example 1. The results indicate that particle size and colloidal stability are important features for the in vivo use in therapeutics.

FIG. 2 shows the high resolution of cellular-nanoparticle interaction imaging in vivo. Left—50 nm and 25 nm mesoporous silica nanoparticles flowing in chick chorioallantoic (CAM) vasculature. Right—50 nm SP94 peptide particles flowing the CAM and bound to a target cancer cell. As determined in the experiment(s) of Example 1.

FIG. 3 shows that targeting of ligands increases the cell specific accumulation of MSNPs in vivo. Modification of surface chemistry (aminated), charge (cationic) and targeted ligand. Targeted with anti-EGFR affibody. As determined in the experiment(s) of Example 1.

FIG. 4 shows an in vivo targeting attempt using PEI and EGFR. As determined in the experiment(s) of Example 1.

FIG. 5 shows a cell targeting attempt which evidences that the number or concentration of particles will influence the ability of the MDNP to target cells. As determined in the experiment(s) of Example 1.

FIG. 6 shows targeted particle binding. 20 ug/ml EGFT Affibody particles (50 nm) with A431 cells. As determined in the experiment(s) of Example 1.

FIG. 7 shows the in vivo tumor targeting of MSNPs (PEG/NMe₃⁺) made pursuant to the present invention. The figure shows the biodistribution of IV injected NIR dye-labeled 50 nm PEG-NMe₃⁺ co-modified MSNP to the BxPC3-luc tumor xenograft model in nude mice with or without TGFβi treatment. As determined in the experiment(s) of Example 1.

FIG. 8 shows SP94 targeted MSNPs in vivo. As determined in the experiment(s) of Example 1.

FIG. 9 shows the binding of 50 nm CD19 Ab nanparticles with certain identified cells. As determined in the experiment(s) of Example 1.

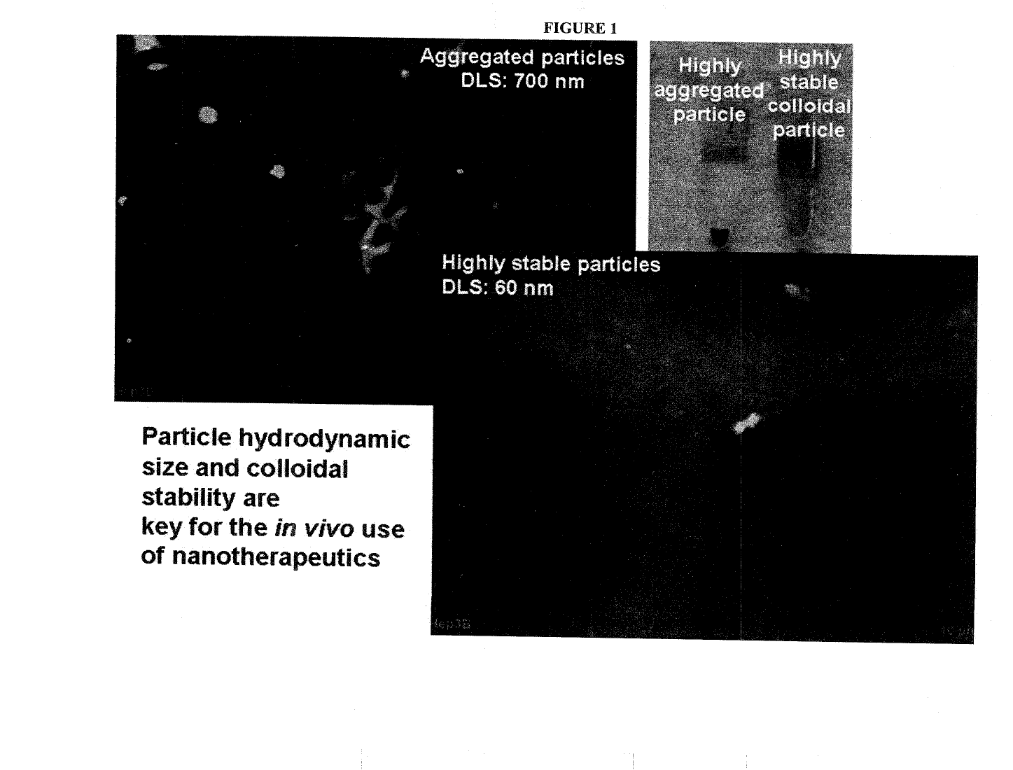

FIG. 1A shows the importance of size in altering the rate and location of deposition of MSNPs according to the present invention in blood vessels in vivo. As determined in the experiment(s) of Example 2. The results evidence that size alters rate and location of deposition. The deposition of particles in low pressure/turbulent vessels appear to correspond to the pattern observed in mice. Flow patterns are an important factor which also influences the probability of contact and deposition which translates to low pressure/velocity/path transitions in organs (e.g. liver, spleen, bone marrow).

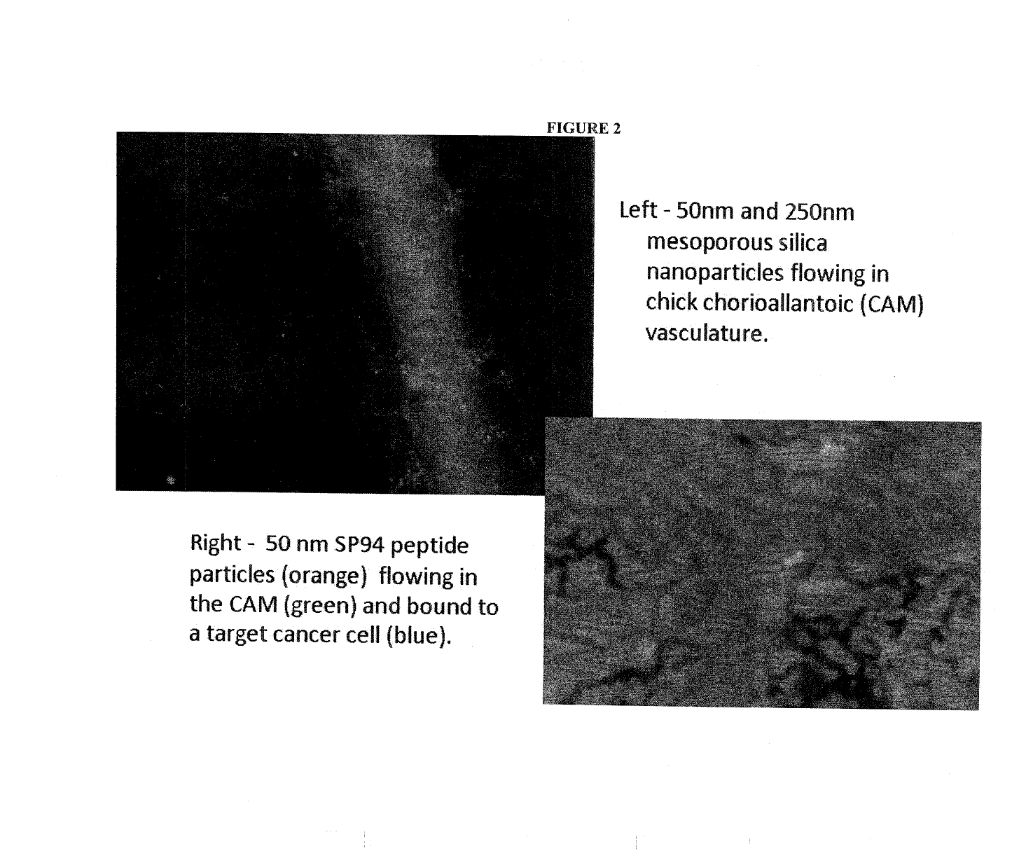

FIG. 2A shows that the cationic charge exposure of the MSNPs modifies the rate and location of the binding of the MSNPs. As determined in the experiment(s) of Example 2. Figure similar to FIG. 1AX.

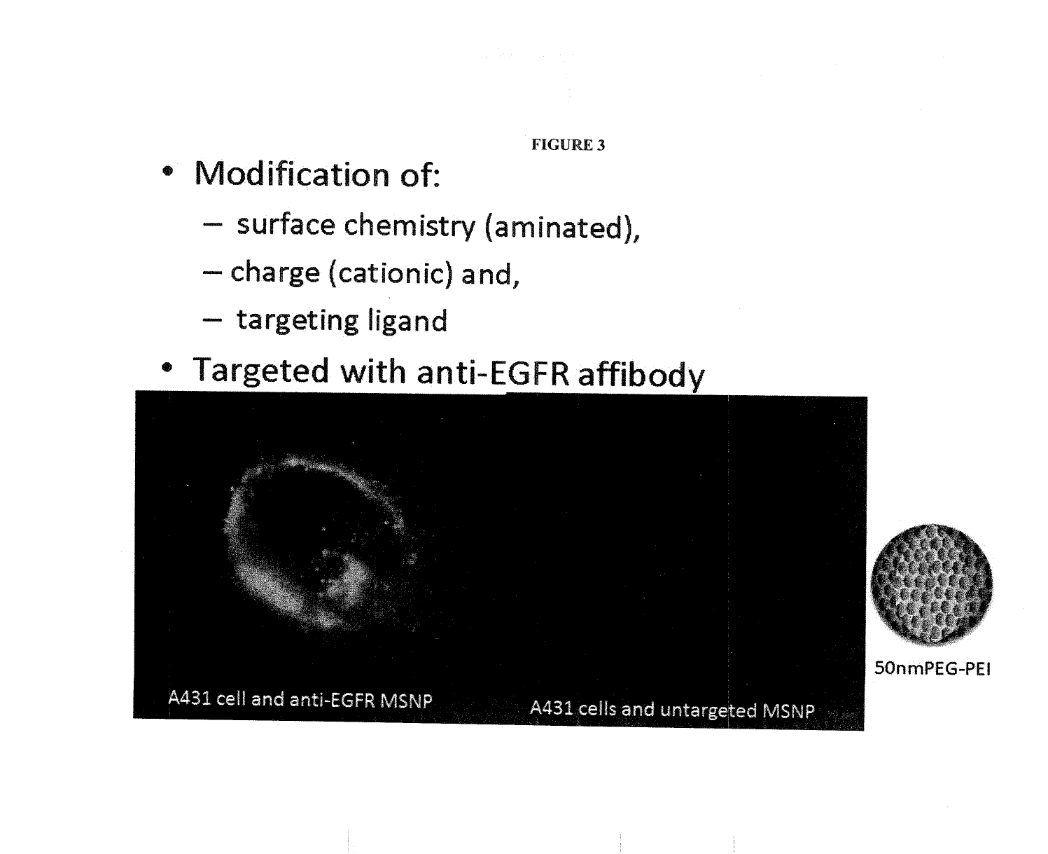

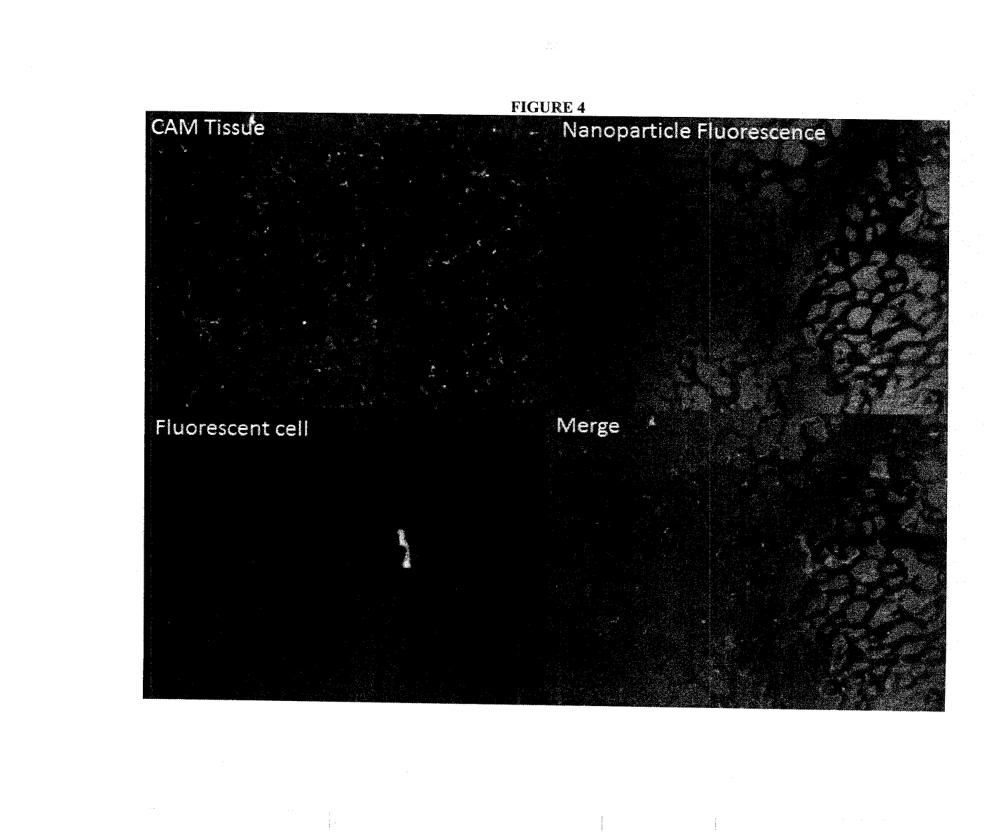

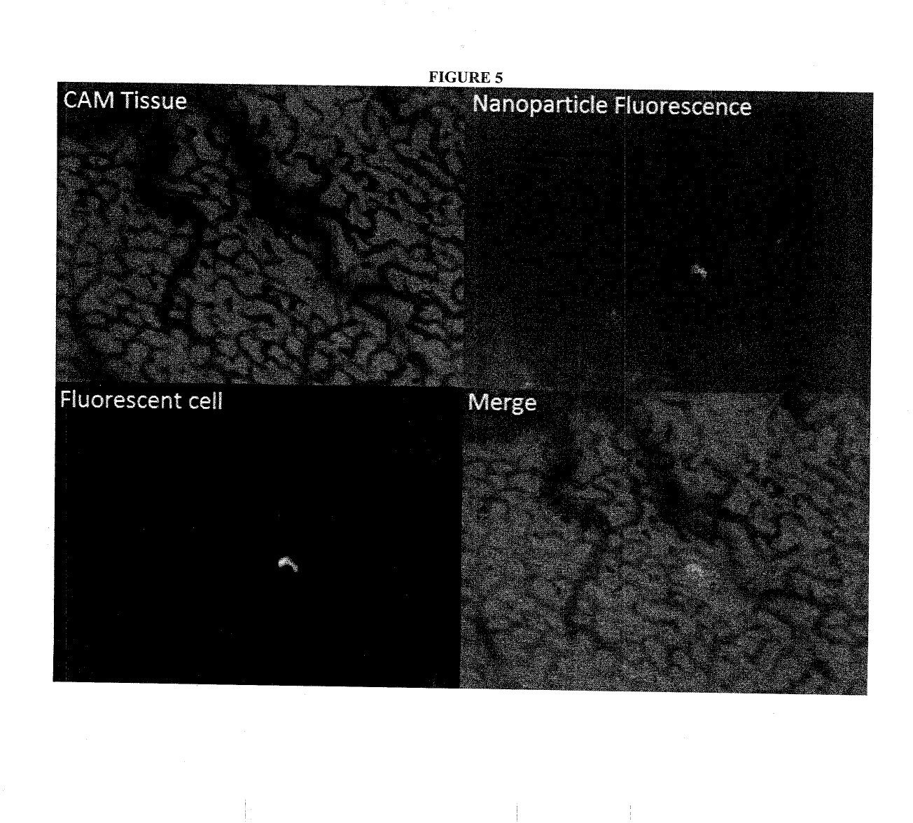

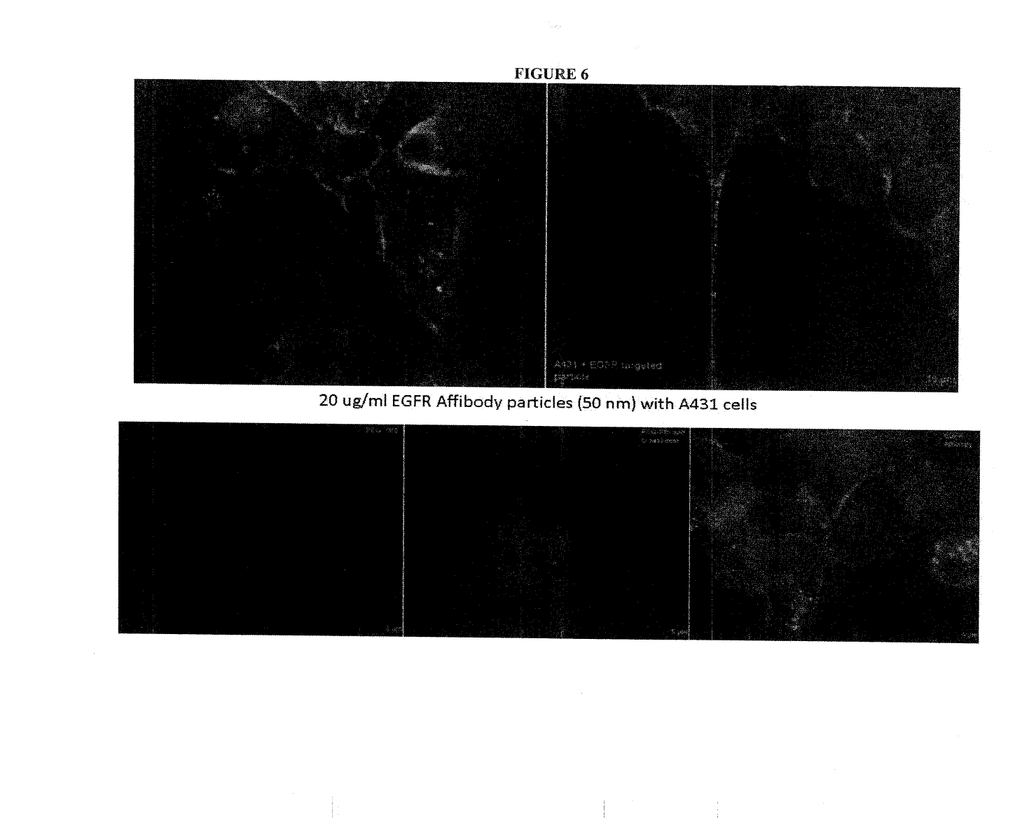

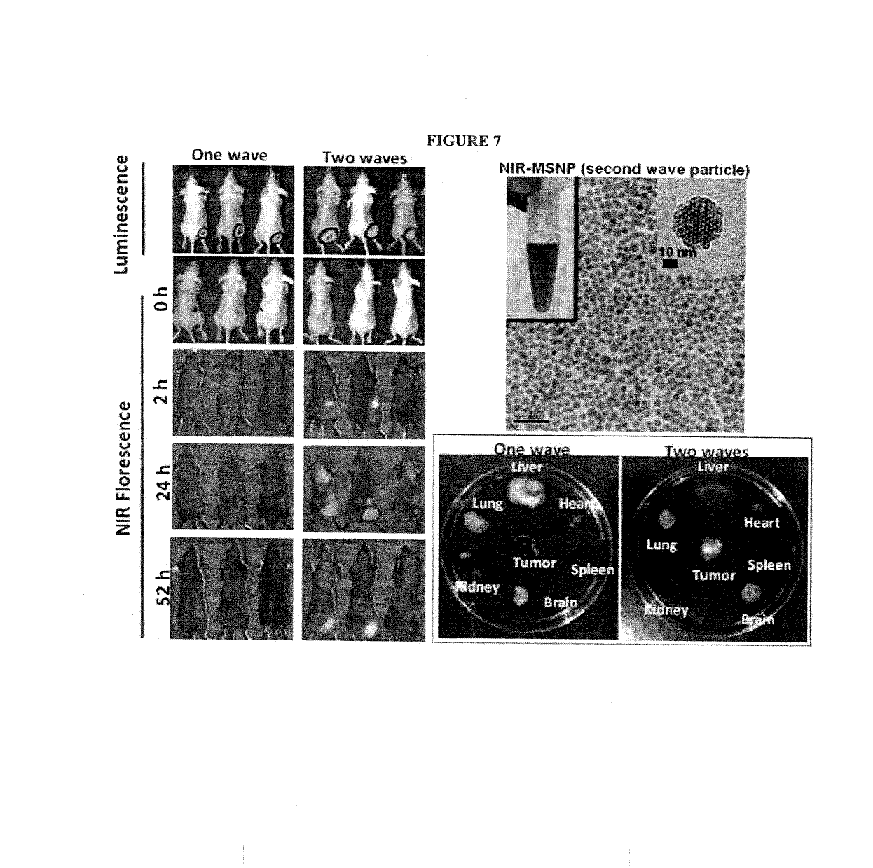

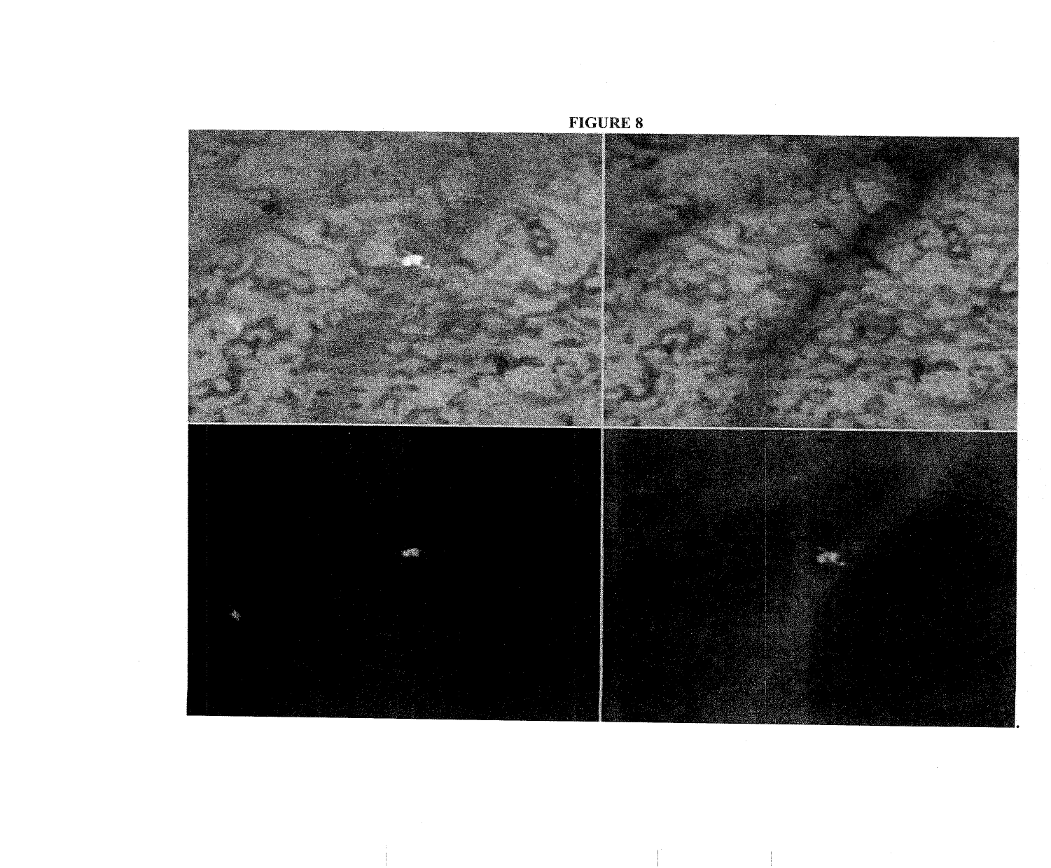

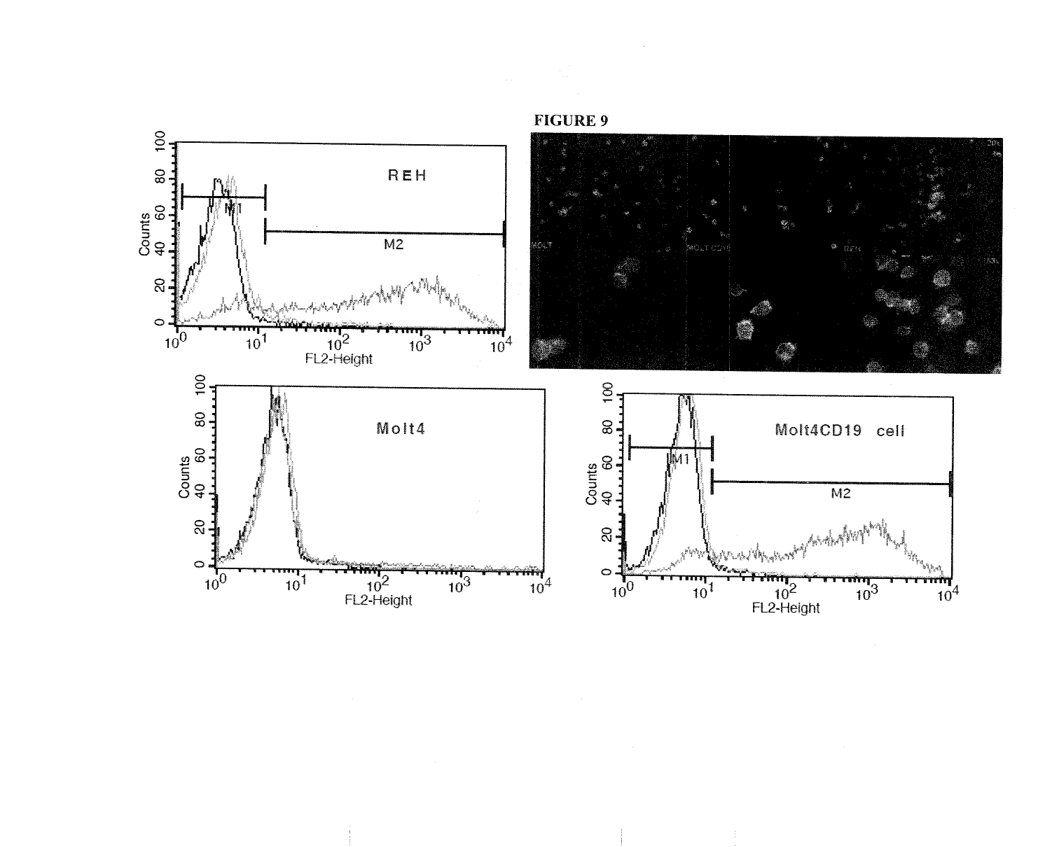

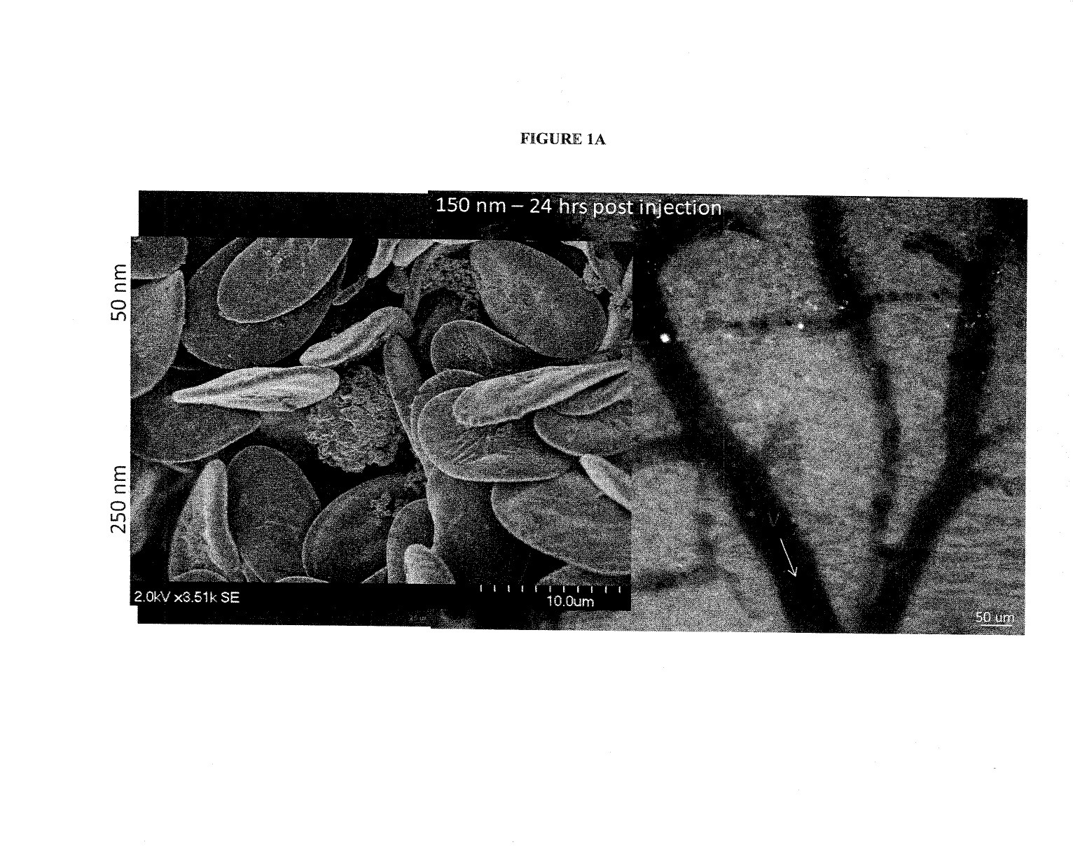

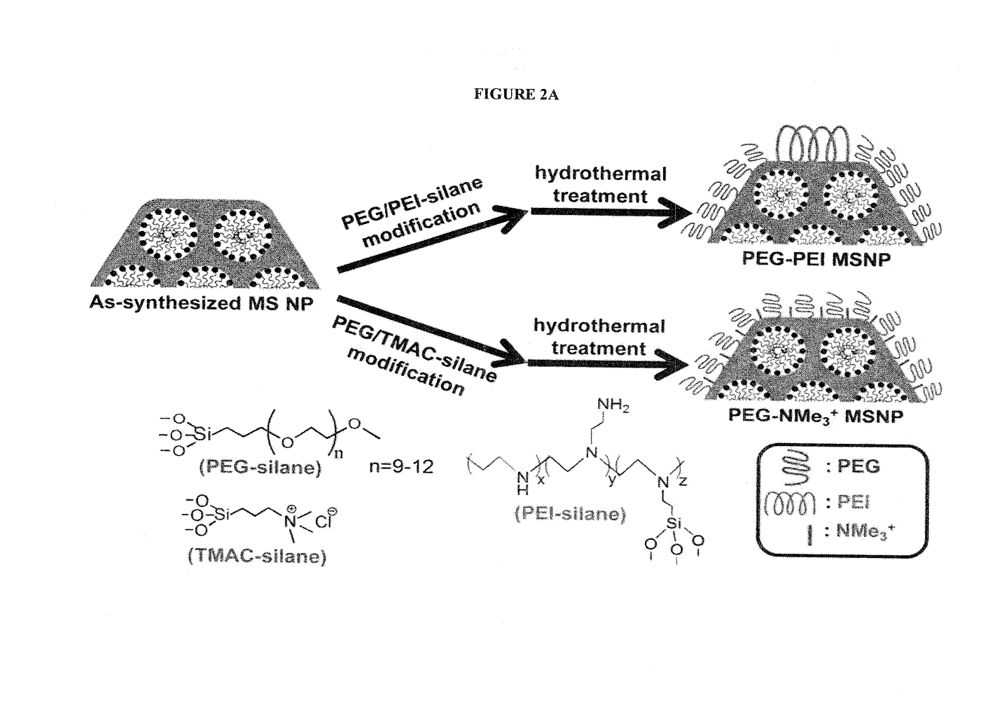

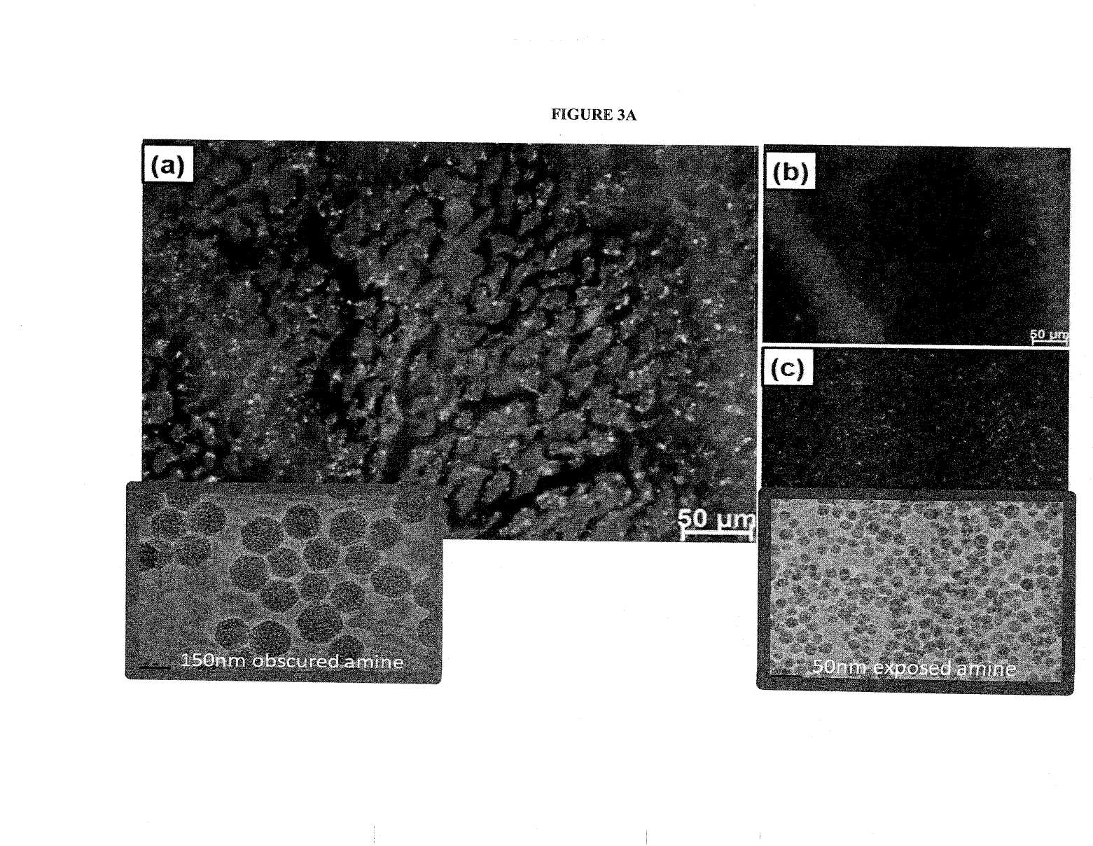

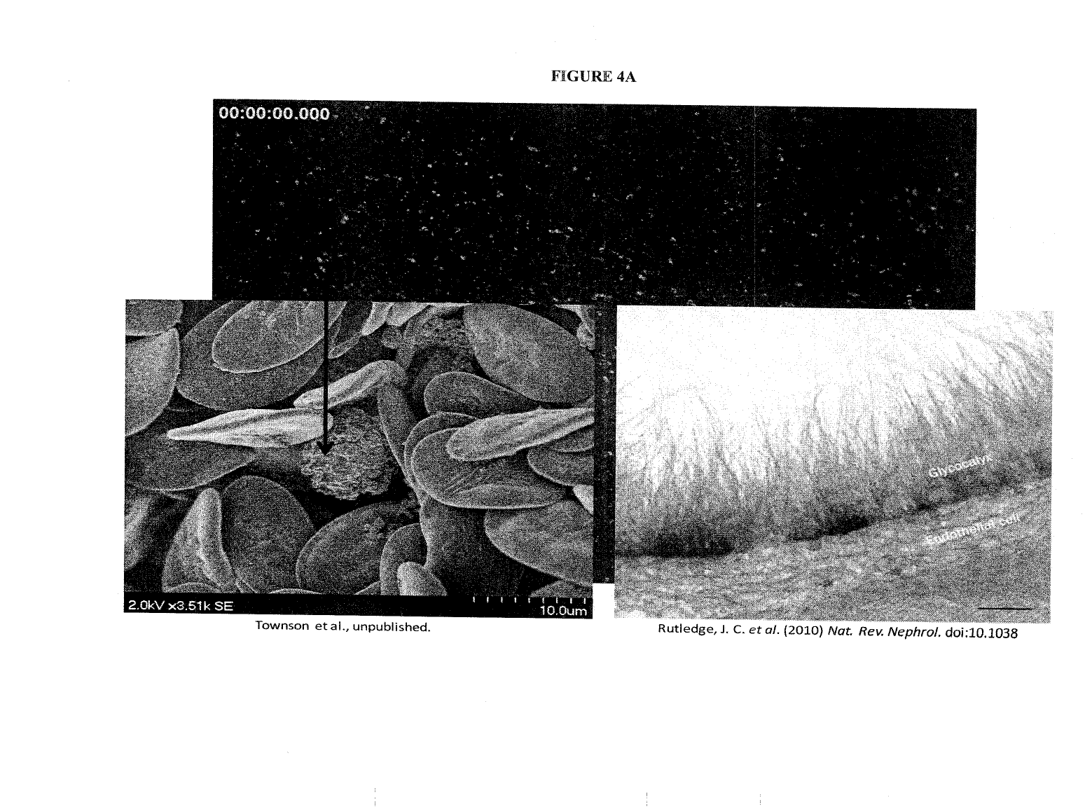

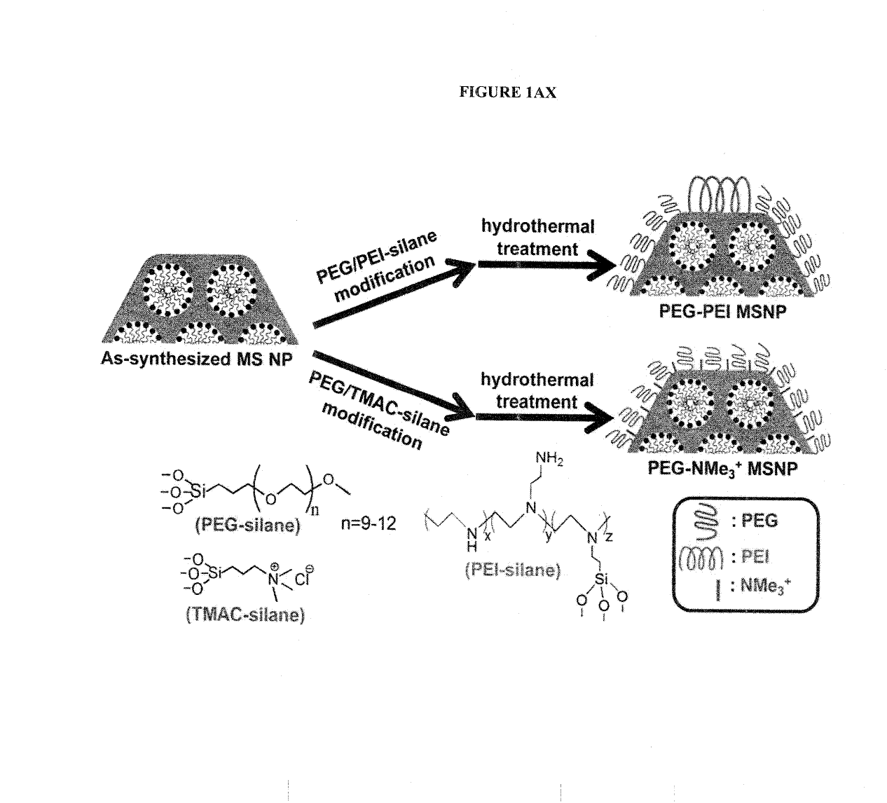

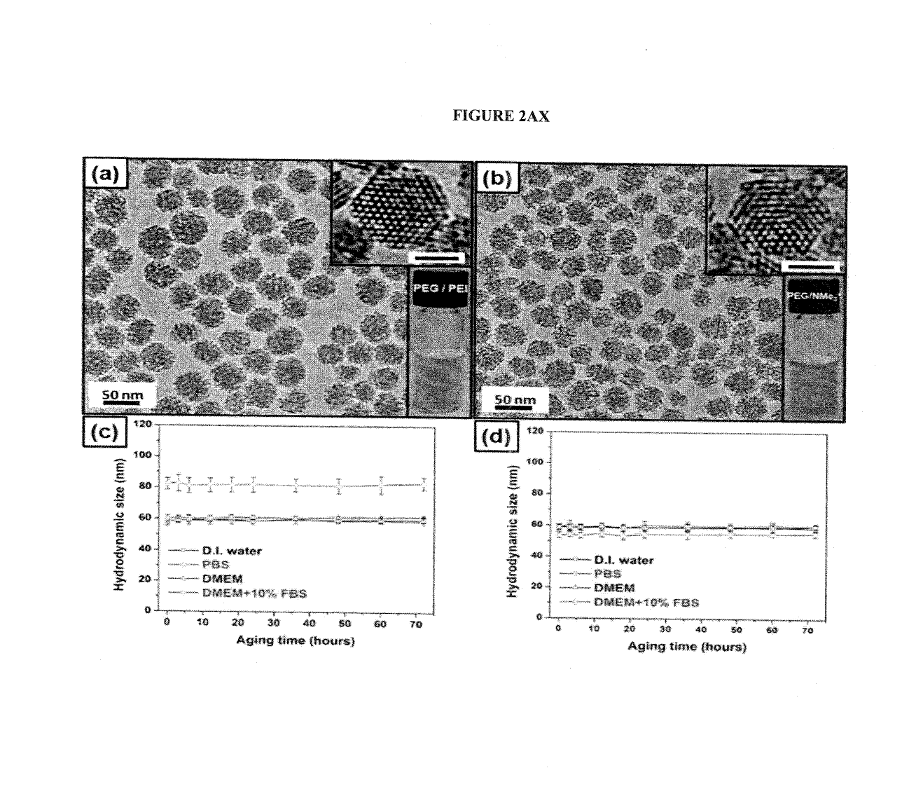

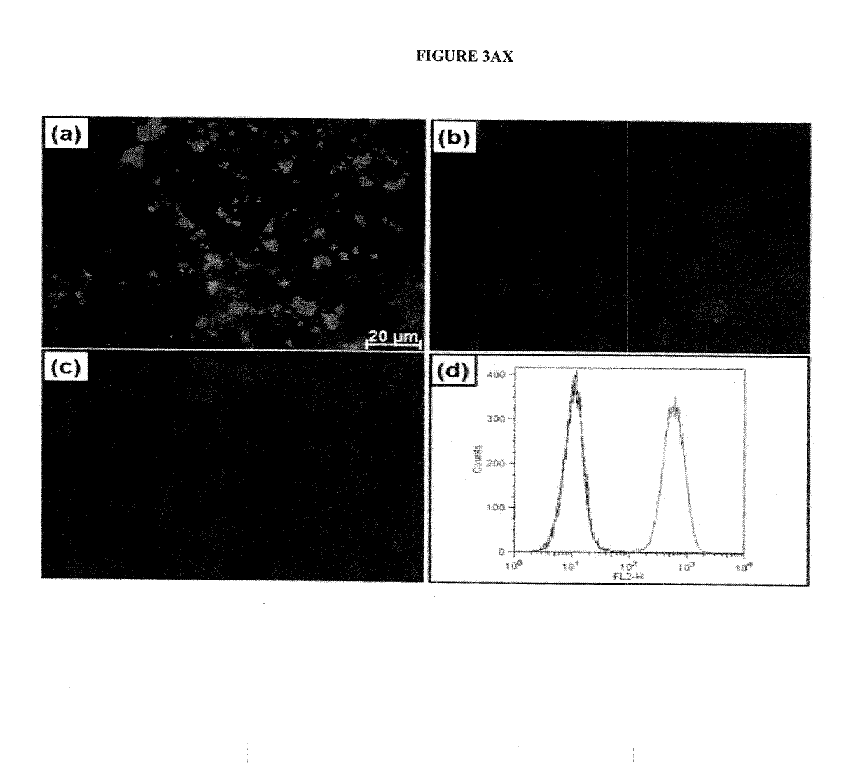

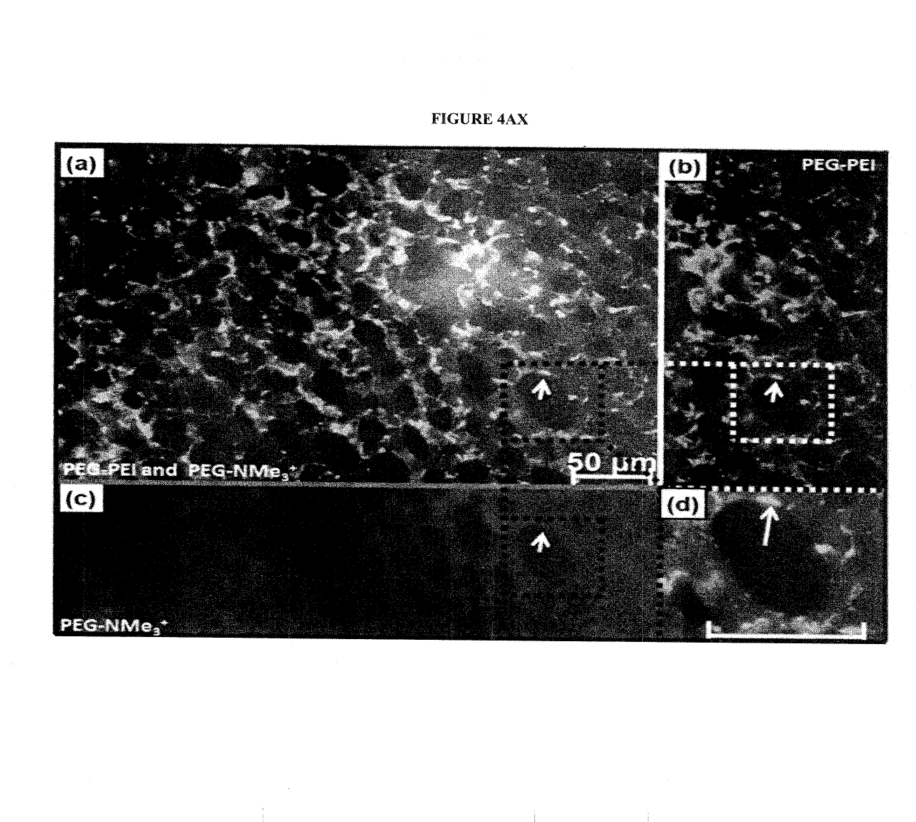

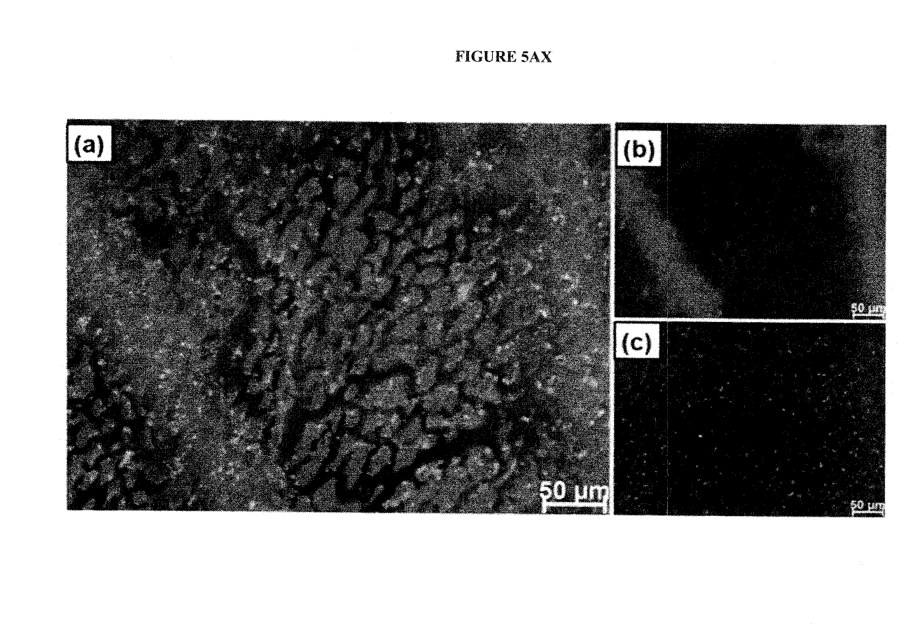

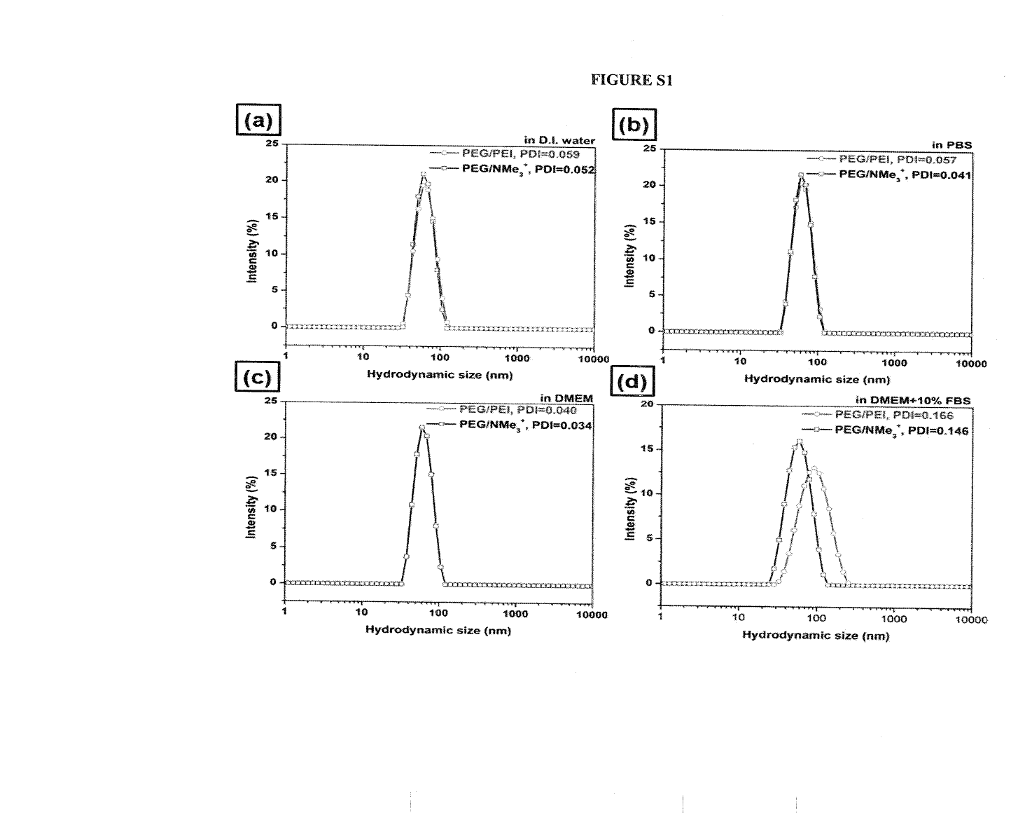

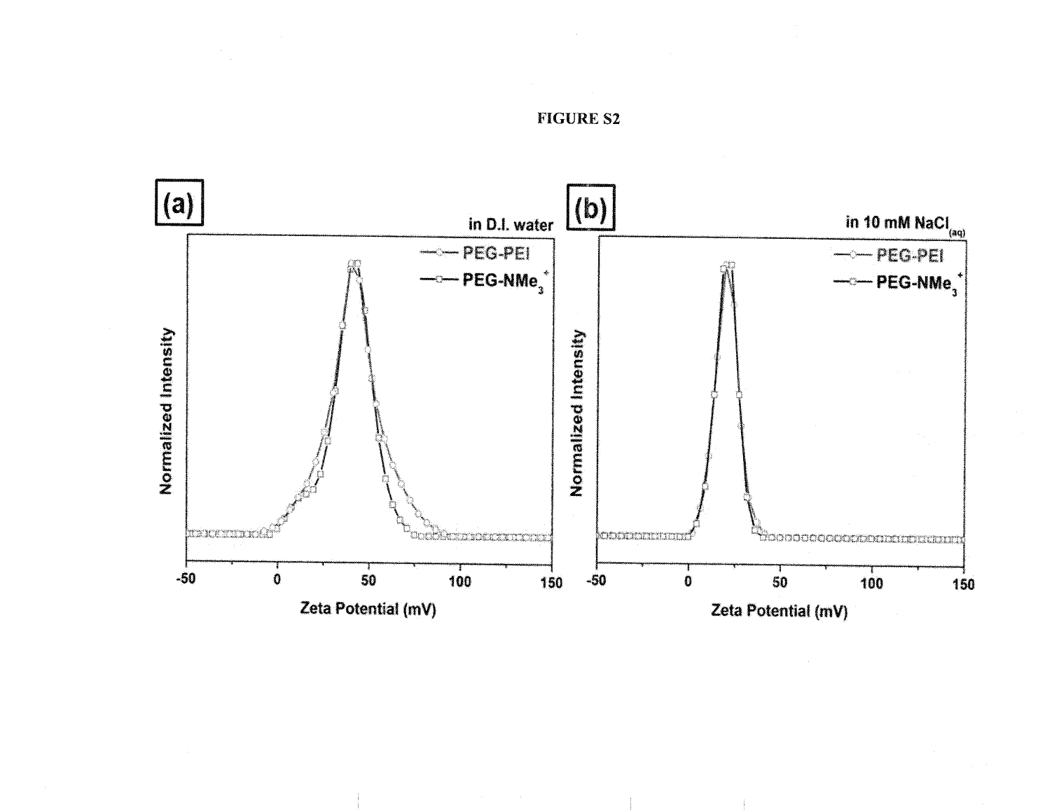

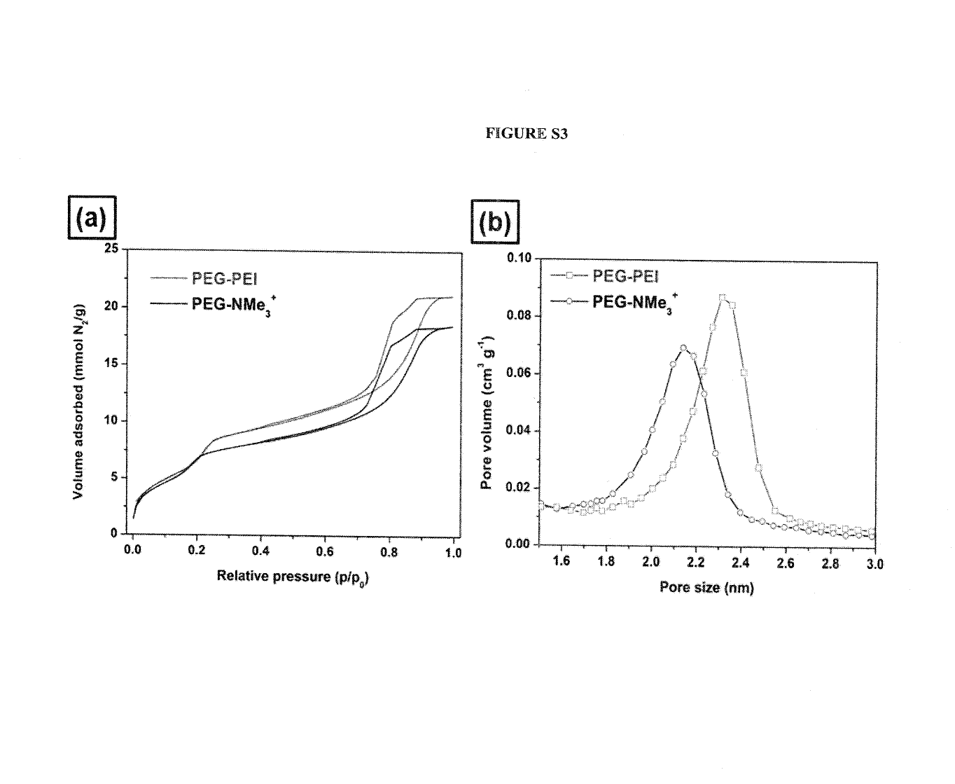

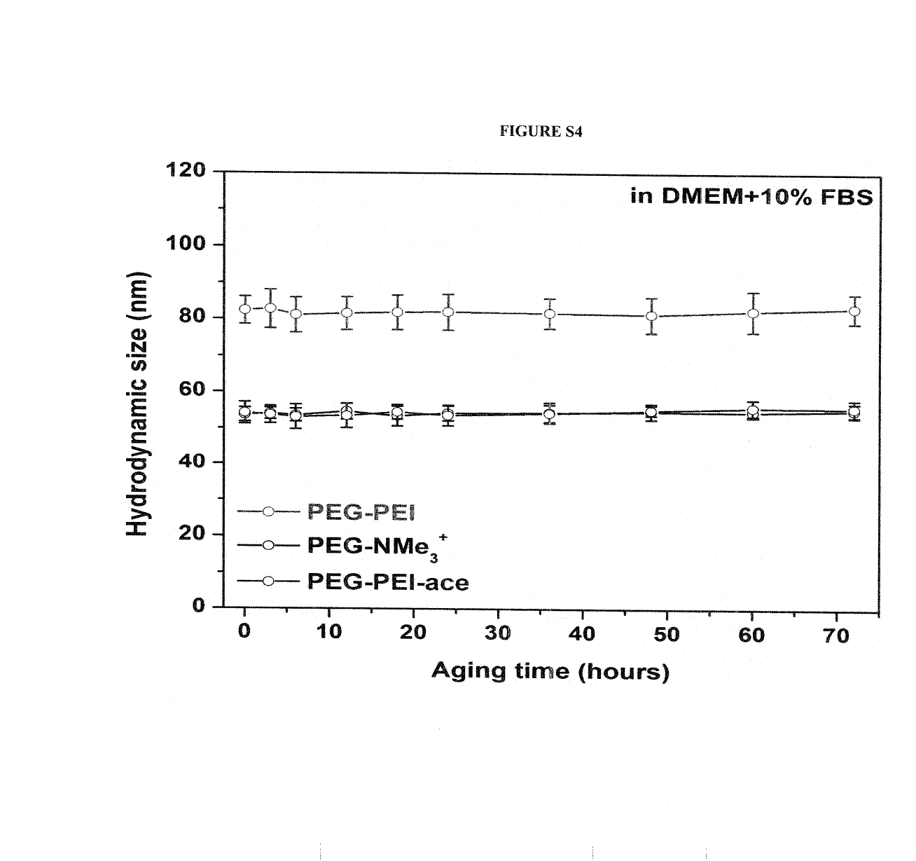

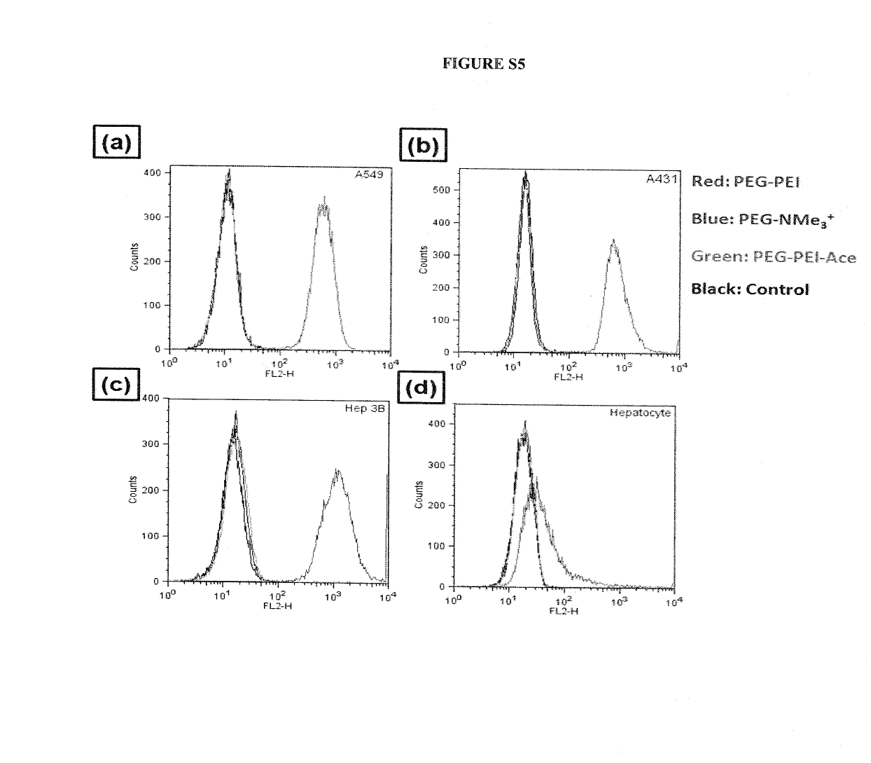

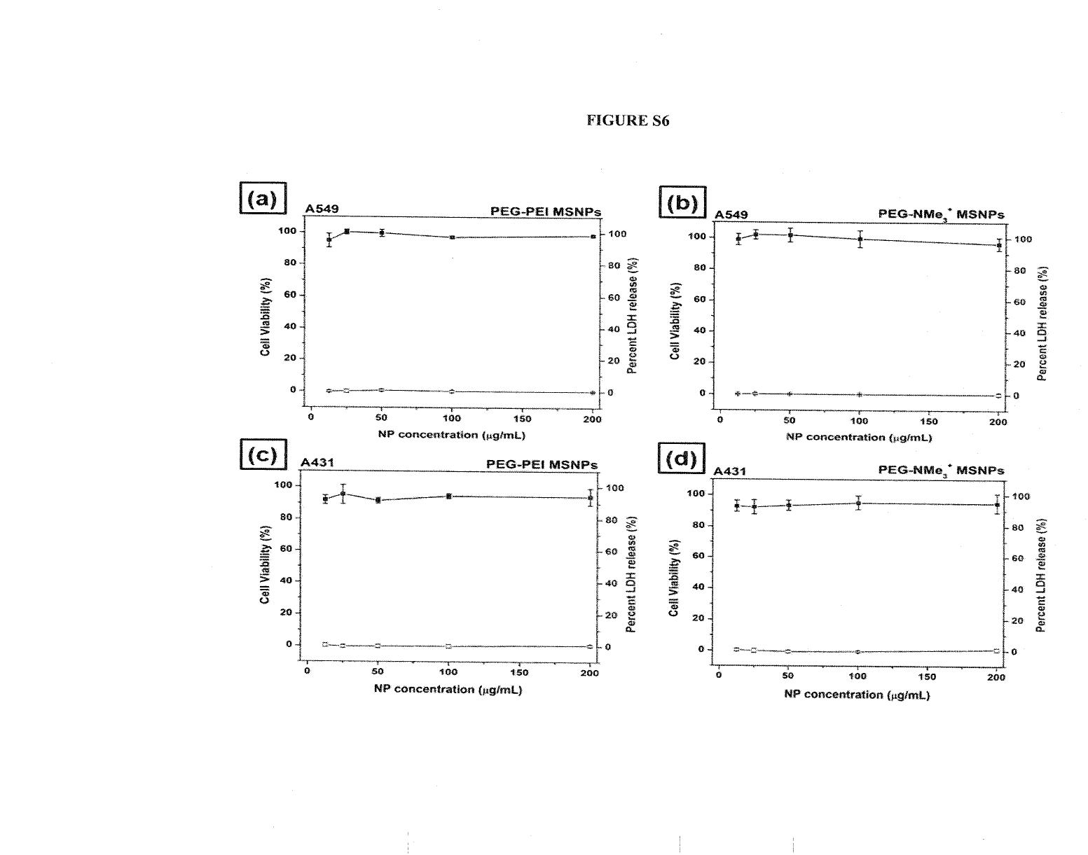

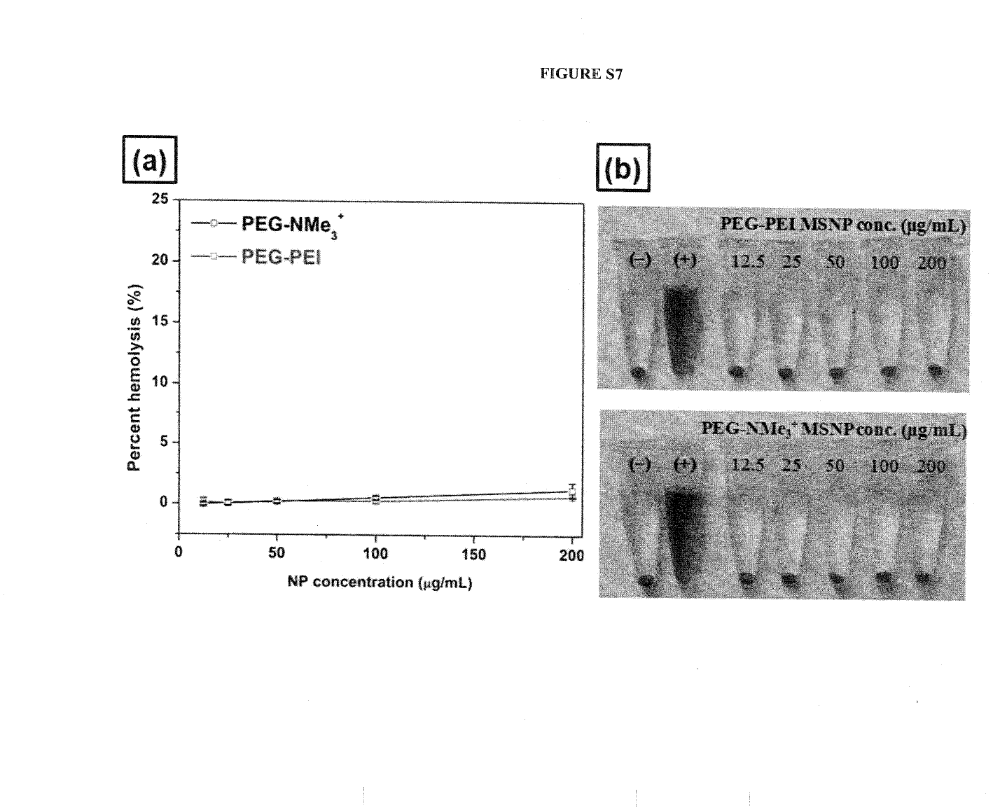

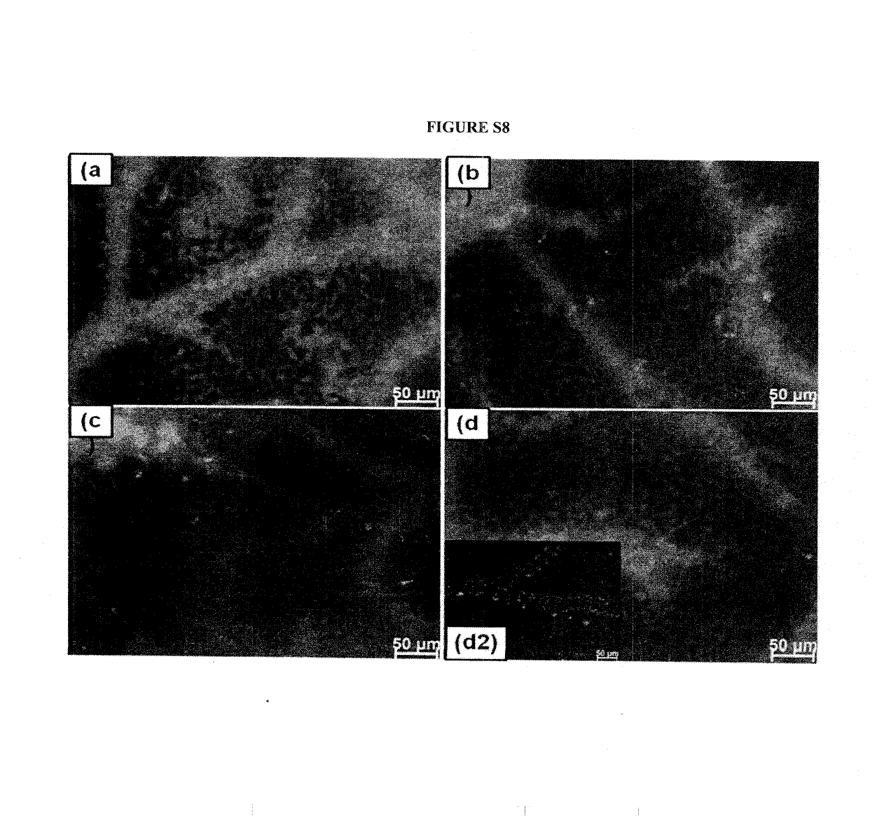

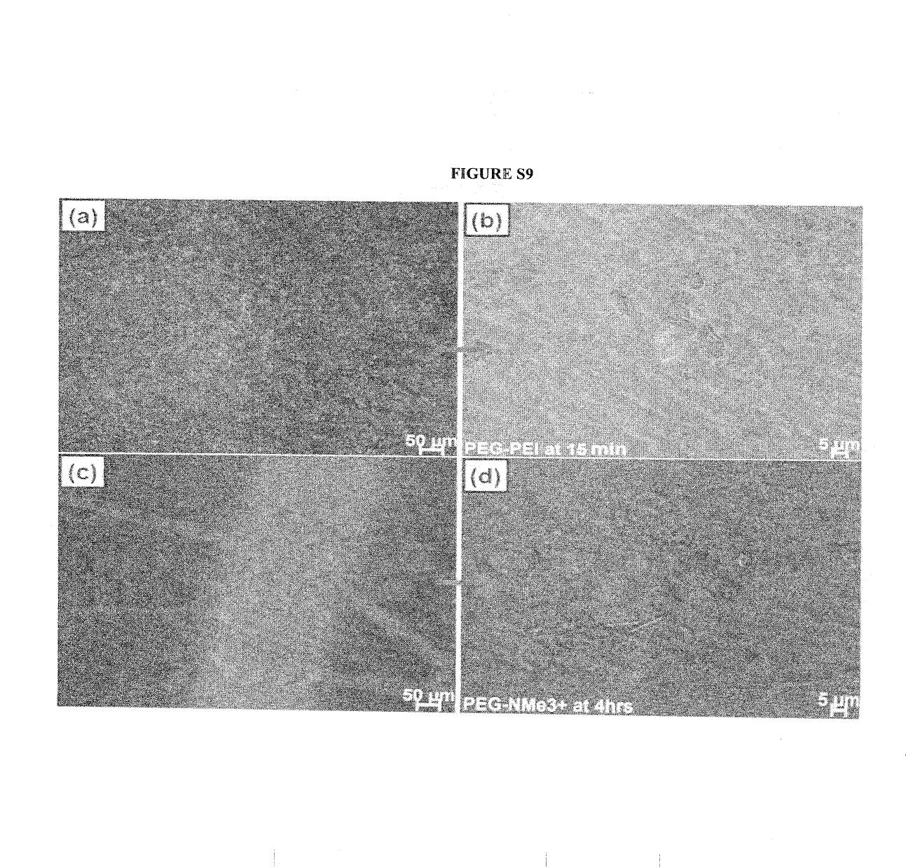

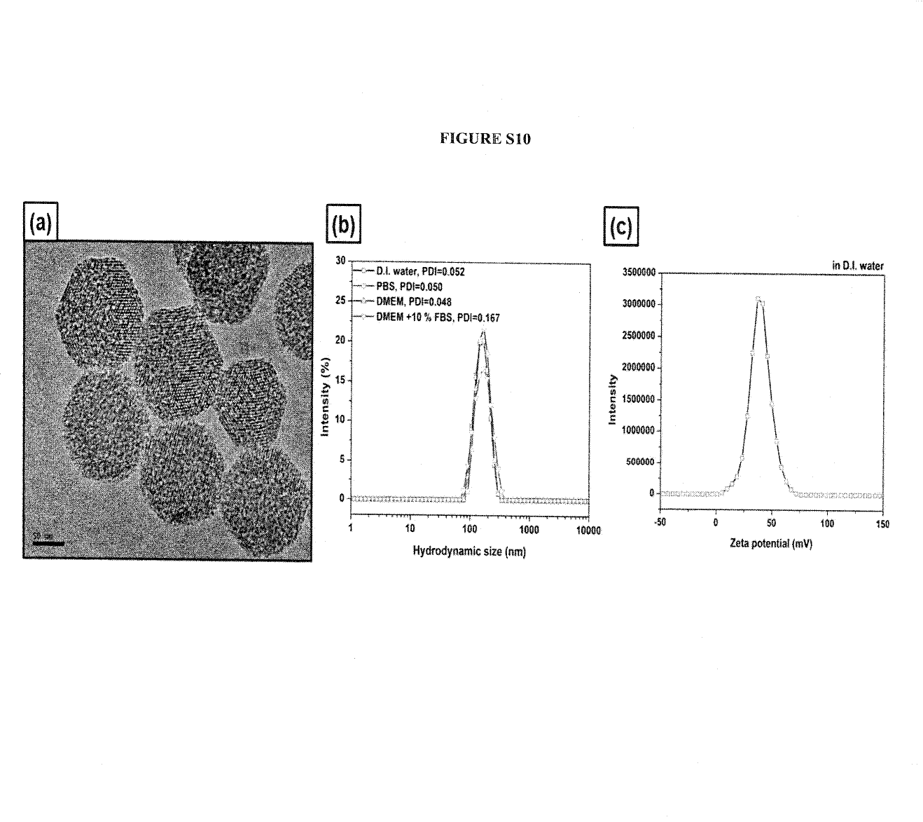

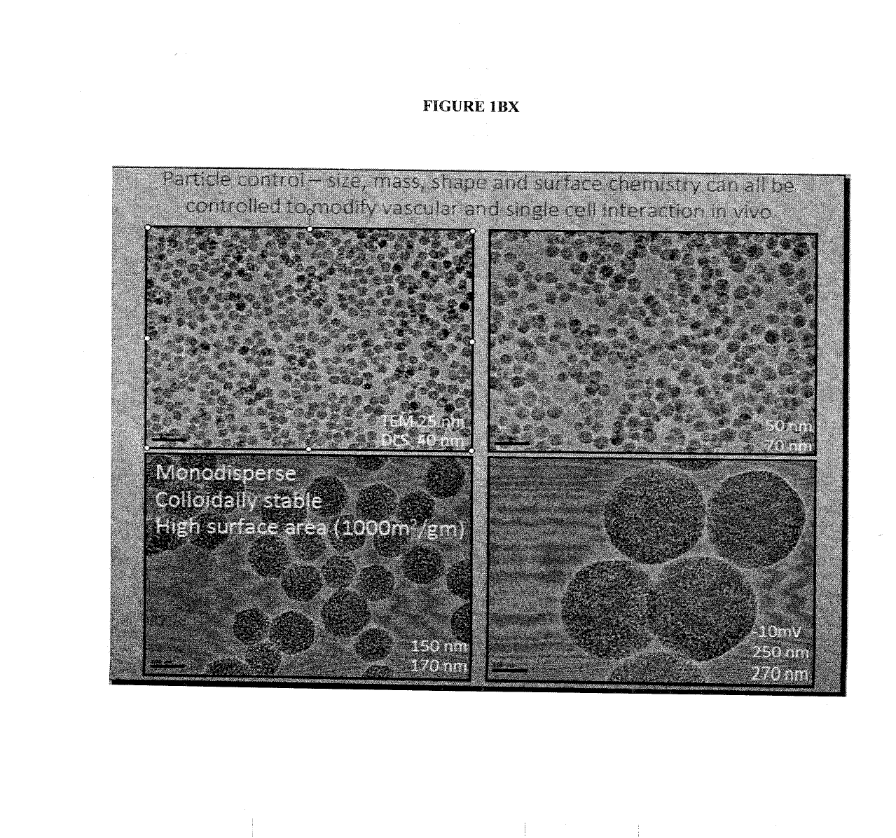

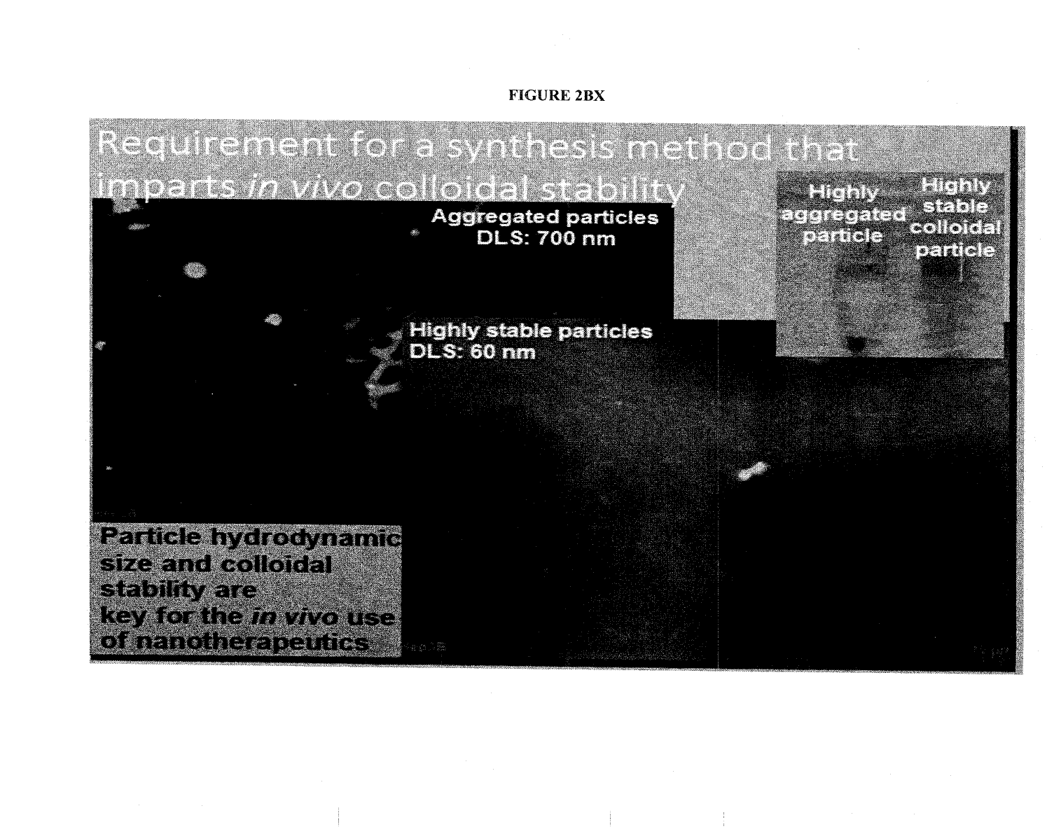

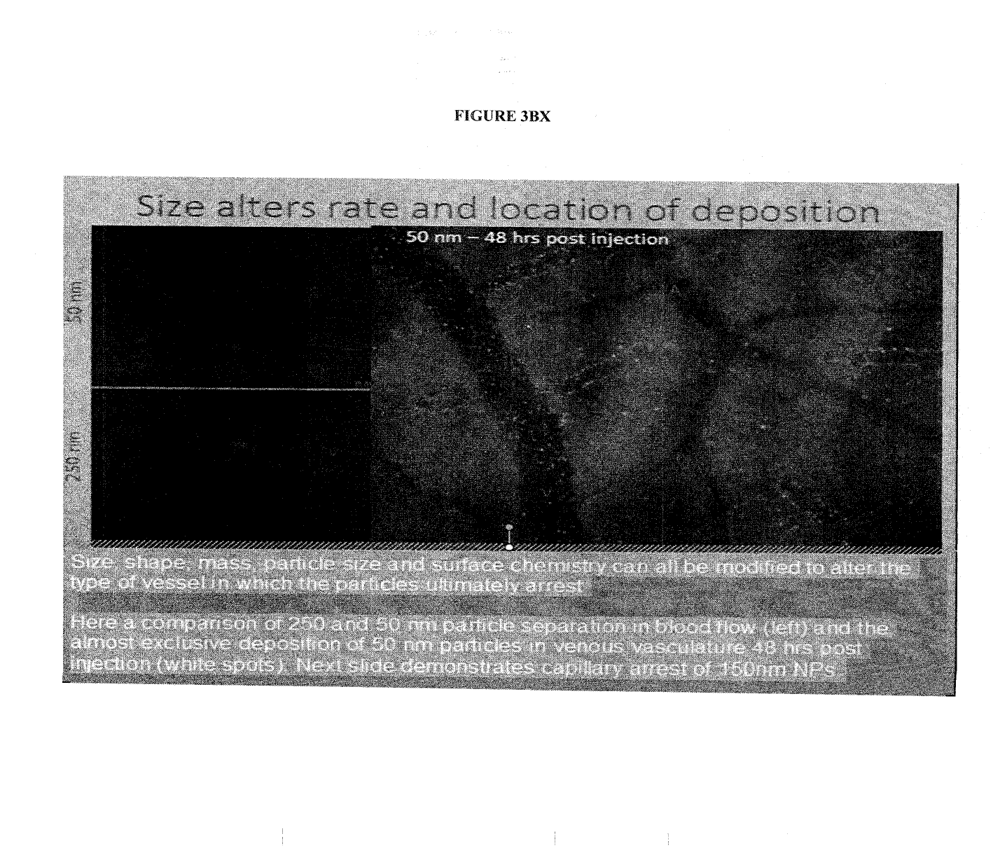

FIG. 3A shows the charge exposure of the MSNPs relative to the size of the particles. As determined in the experiment(s) of Example 2.

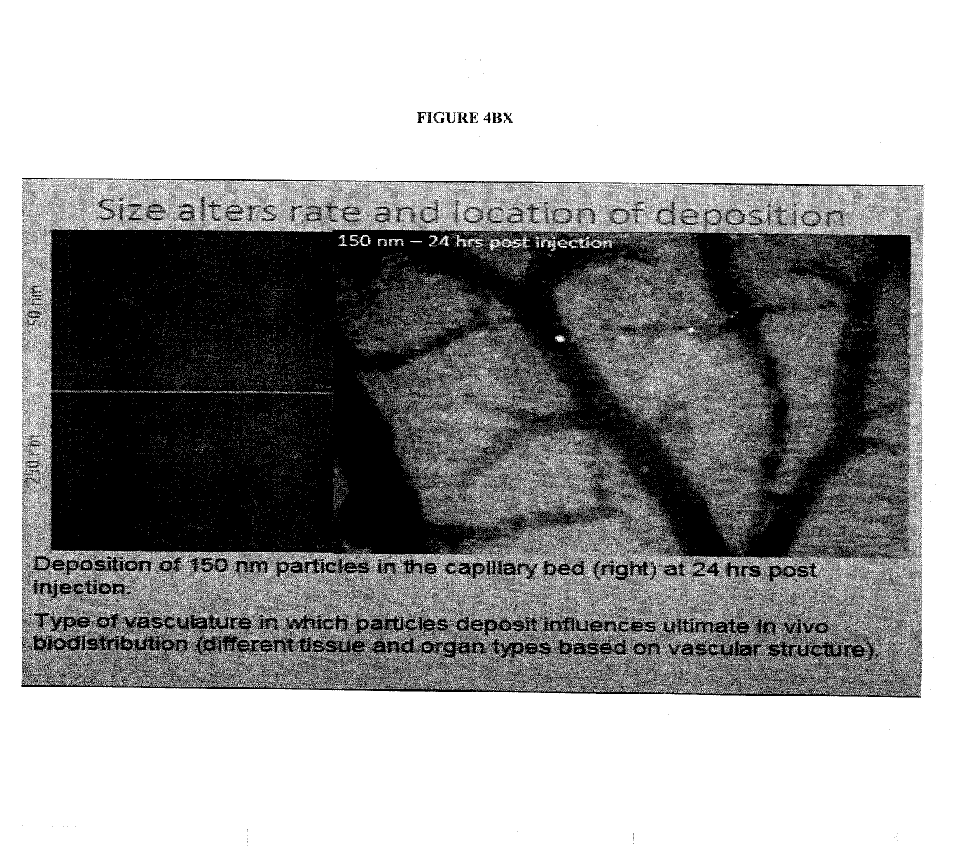

FIG. 4A shows an in vivo targeting attempt using PEI and EGFR. As determined in the experiment(s) of Example 2. Endothelial and white blood cell binding is exhibited.

FIG. 1AX. Schematic illustration of the design and synthesis of two positively charged PEGylated MSNPs, designated as PEG-PEI and PEG-NMe3+. As determined in the experiment(s) of Example 3.

FIG. 2AX. (a,b) TEM and optical images of rhodamine B-labeled PEGPEI (a) and PEG-NMe3+ (b) MSNPs following removal of surfactant, scale bar=25 nm. (c, d) Hydrodynamic diameters of PEG-PEI (c) and PEG-NMe3+ (d) MSNP vs aging time in various solutions. As determined in the experiment(s) of Example 3.

FIG. 3AX. Differential binding of red fluorescently labeled PEG-PEI, PEG-NMe3+, and PEG-PEI-ace particles to A549 cells in vitro. PEG-PEI (a) but not PEG-NMe3+ (b) or PEG-PEI-ace (c) particles are observed to bind to A549 cells (blue—dapi stained nuclei) via fluorescent microscopy. Particle binding is confirmed by flow cytometry (d). PEGPEI (red), PEG-NMe3+ (blue), PEG-PEI-ace (green), and cells only (black). As determined in the experiment(s) of Example 3.

FIG. 4AX. Differential in vivo binding and flow of size- and charge-matched PEG-PEI (orange) and PEG-NMe3+ (green) MSNP in chickCAM 10-min post injection. (a) Merged image, (b) PEG-PEI showing arrest on endothelial cells, and (c) PEG-NMe3+ image showing circulating MSNPs. (d) Magnification of PEG-PEI MSNP binding (arrow) on endothelial cells (scale bar 50 μm). As determined in the experiment(s) of Example 3.

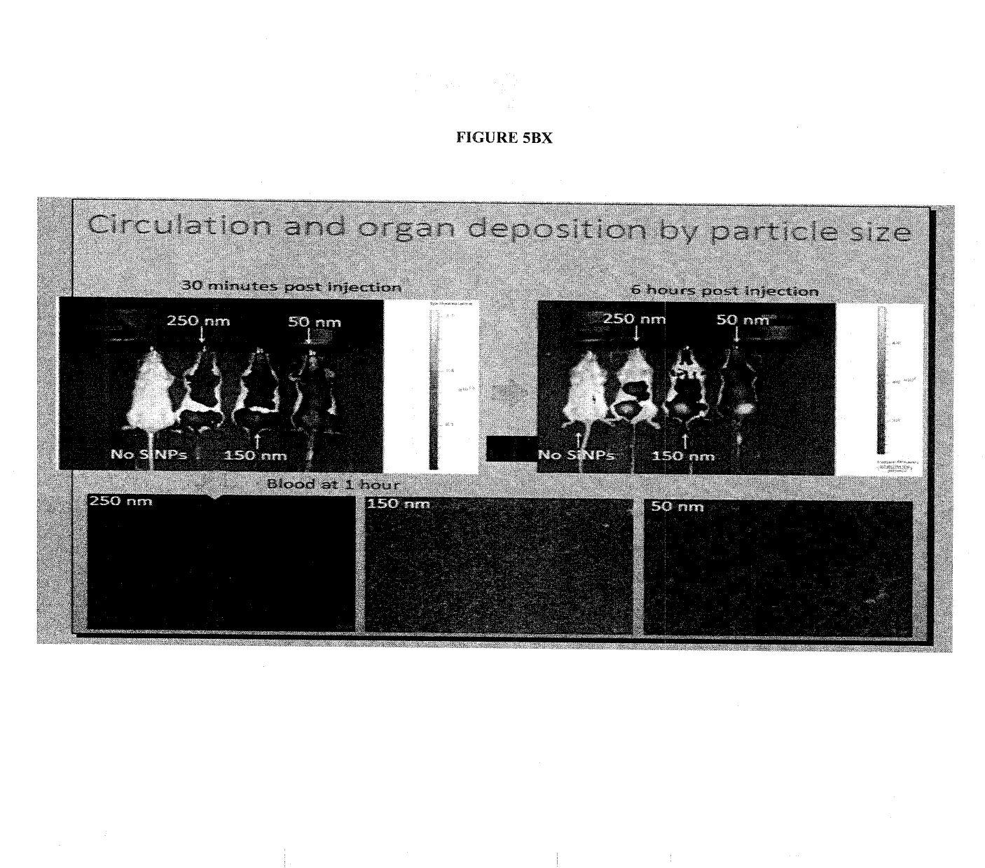

FIG. 5AX. Size vs exposed amine-mediated binding. PEG-PEI MSNPs, 50 nm (green, +40 mV), were observed to bind to endothelial cells immediately after injection. PEG-NMe3+MSNPs, 150 nm (red, +40 mV), circulated for hours post injection. (a) Merged image, (b) 150 nm PEG-NMe3+, (c) 50 nm PEG-PEI. (The field of view of b and c is the same as that of a.) As determined in the experiment(s) of Example 3.

FIG. S1. Representative hydrodynamic size distribution of PEG-PEI and PEG-NMe₃⁺ MSNPs (1 mg/mL) measured at RT in various solutions: (a) D.I. water, (b) PBS, (c) DMEM, and (d) DMEM+10% FBS.

FIG. S2. Surface charge distribution of PEG/PEI and PEG/NMe₃⁺ MSNPs in (a) D.I. water and (b) 10 mM NaCl_{(aq)}. As determined in the experiment(s) of Example 3.

FIG. S3. (a) N₂ adsorption-desorption isotherms and (b) BJH pore size distribution of PEG-PEI and PEG-NMe₃⁺ MSNPs. As determined in the experiment(s) of Example 3.

FIG. S4. Long term particle stability of PEG-PEI, PEG-NMe₃⁺, and acetylated PEG-PEI MSNPs. As determined in the experiment(s) of Example 3.

FIG. S5. Flow cytometry of (a) A549, (b) A431, (c) Hep3B, and (d) hepatocyte cells after 30 min incubation to 10 μg/mL of PEG-PEI and PEG-NMe₃⁺ MSNPs at 37° C. under 5% CO₂. As determined in the experiment(s) of Example 3.

FIG. S6. Cell viability of A549 (a,b) and A431 (c,d) cells determined from WST-8 and LDH assay after 24 h exposure to various concentrations (12.5, 25, 50, 100, and 200 μg/mL) of PEG-PEI and PEG-NMe₃⁺ MSNPs. Data represent mean±SD (n=4). As determined in the experiment(s) of Example 3.

FIG. S7. (a) Percentage of lysed human red blood cells (hRBCs) after exposure to 12.5, 25, 50, 100, and 200 μg/mL) of PEG-PEI and PEG-NMe₃⁺ MSNPs for 2 h at 37° C. Data represent mean±SD (n=3). (b) Digital photographs of hRBCs after 2 h incubation to PEG-PEI and PEG-NMe₃⁺ MSNPs at different concentrations (12.5 to 200 μg/mL). The presence of red hemoglobin in the supernatant indicates membrane damaged hRBCs. The (−) and (+) symbols represent positive control and negative control, respectively. As determined in the experiment(s) of Example 3.

FIG. S8. Vascular circulation of PEG-NMe₃⁺ and PEG-PEI-Ace particles over time. (a-c) PEG-NMe₃⁺ MSNPs (a) immediately, (b) 1 h and (c) 6 h post injection. Note in all cases particles are primarily circulating and vasculature with little binding to endothelium observed. (d) PEG-PEI-Ace MSNPs (3 h post injection) continue to circulate with no apparent endothelium binding observed. (d2) 21 h post injection PEG-PEI-Ace particle binding is observed in large venules differing from immediate capillary binding observed with unmodified PEG-PEI particles. As determined in the experiment(s) of Example 3.

FIG. S9. In vivo and ex vivo red and white blood cell-NP interactions. (a) PEG-PEI MSNP binding immediately post injection in CAM. Particles are observed lining the endothelium as well as adhered to 6-8 μm sized cells but not red blood cells. (b) Blood samples removed 15 min post injection of PEG-PEI MSNPs. Few particles observed in solution with most apparent adhered to white blood cells (red spots) but not red blood cells. (c) PEG-NMe₃⁺ particles 3 hrs post injection. (d) Whole blood removed 4 h post injection of PEG-NMe₃⁺ NPs. Particles are apparent in blood with no apparent binding to white or red blood cells. As determined in the experiment(s) of Example 3.

FIG. S10. (a) TEM image of 150 nm PEG-NMe₃⁺ MSNPs. (b) Representative hydrodynamic size distribution of 150 nm PEG-NMe₃⁺ MSNPs (1 mg/mL) measured at RT in various solutions: D.I. water, PBS, DMEM, and DMEM+10% FBS. (c) Zeta potential distribution of 150 nm PEG-NMe₃⁺ MSNPs measured in D.I. water. As determined in the experiment(s) of Example 3.

FIG. 1BX shows monodisperse, colloidally stable MSNPs of the invention having a variety of sizes and charges. As determined in the experiment(s) of Example 4.

FIG. 2BX depicts (1) polydisperse, non-colloidally stable particle shown arresting in vasculature in upper left image (and settled blue pellet in tube in upper right).

(2) (Lower right) Stabilized particles (orange) are observed blowing within the blood vessels and binding to target cancer cells (green). As determined in the experiment(s) of Example 4.

FIG. 3BX shows a comparison of 250 and 50 nm particle separation in blood flow (left) and the almost exclusive deposition of 50 nm particles in venous vasculature 48 hrs post injection (white spots). As determined in the experiment(s) of Example 4.

FIG. 4BX illustrates that particle size alters the rate and location of MSNPs deposition. As determined in the experiment(s) of Example 4.

FIG. 5BX illustrates circulation and organ deposition by particle size. As determined in the experiment(s) of Example 4.

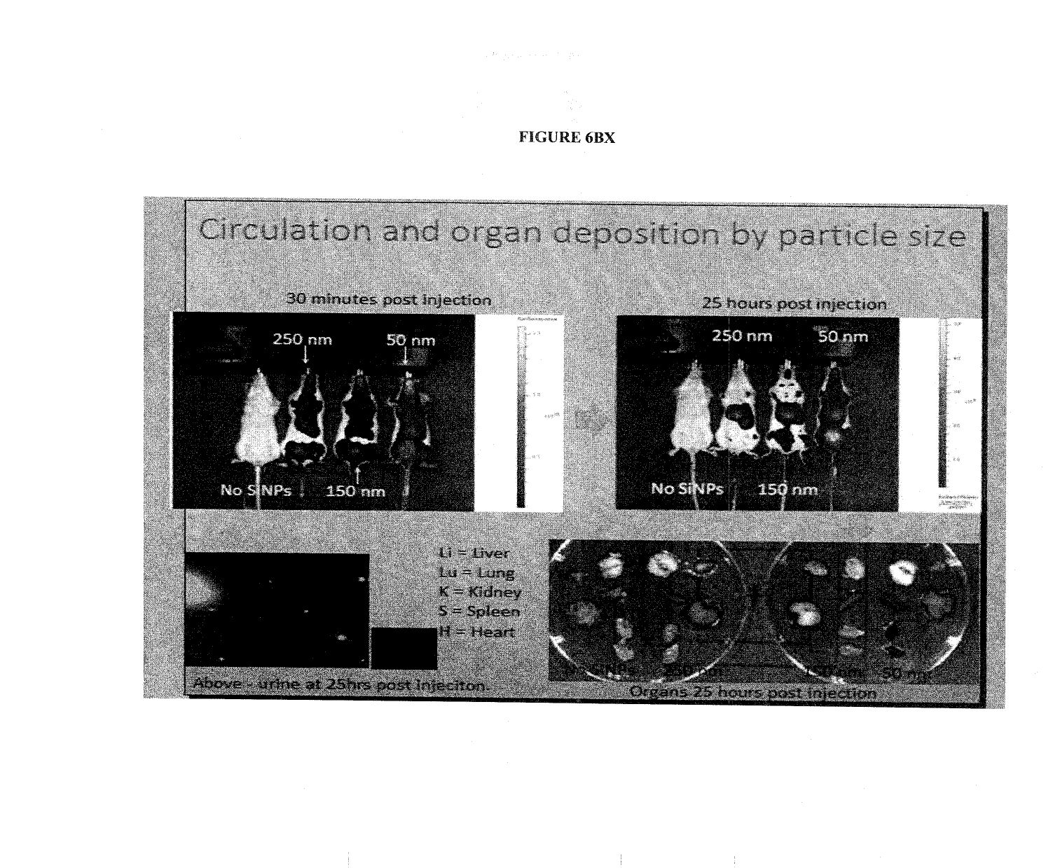

FIGS. 6BX and 7BX also illustrate the relationship of circulation and organ deposition by particle size. As determined in the experiment(s) of Example 4.

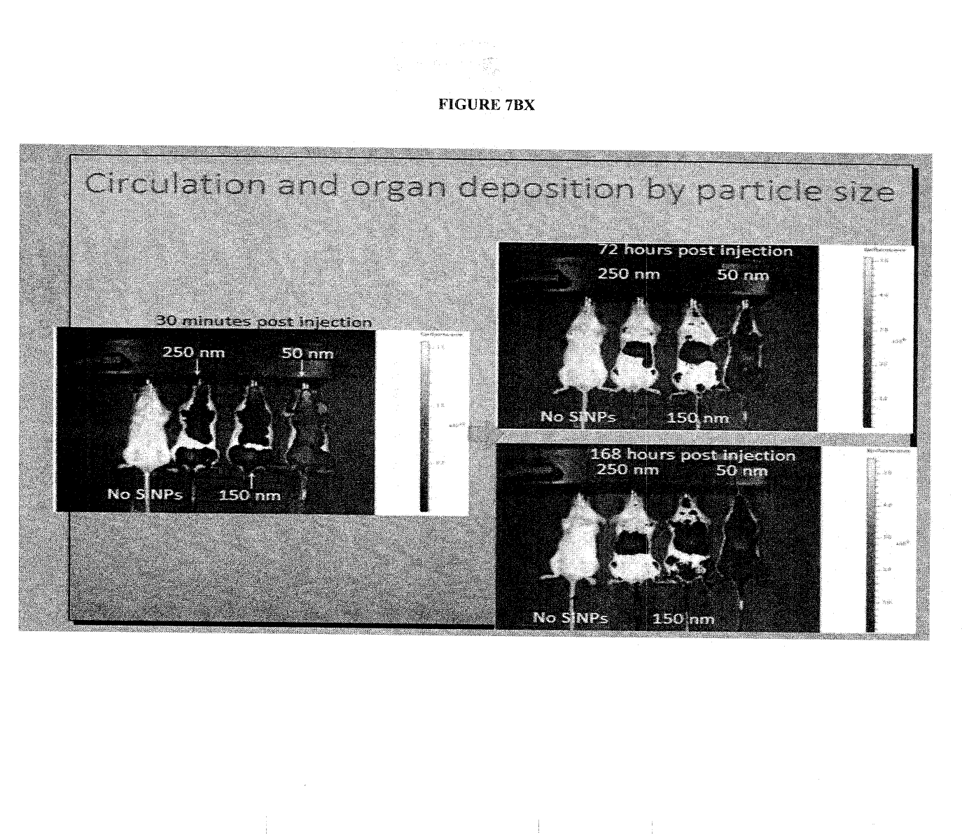

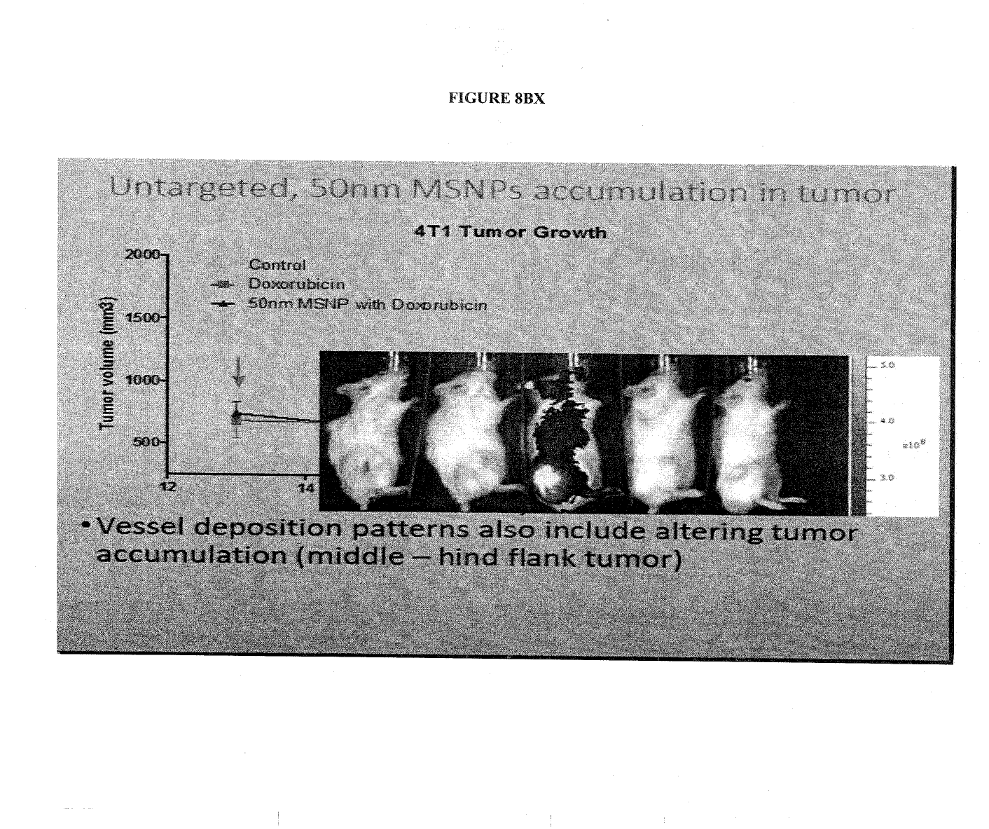

FIG. 8BX shows untargeted 50 nm MSNP accumulation in a tumor. As determined in the experiment(s) of Example 4.

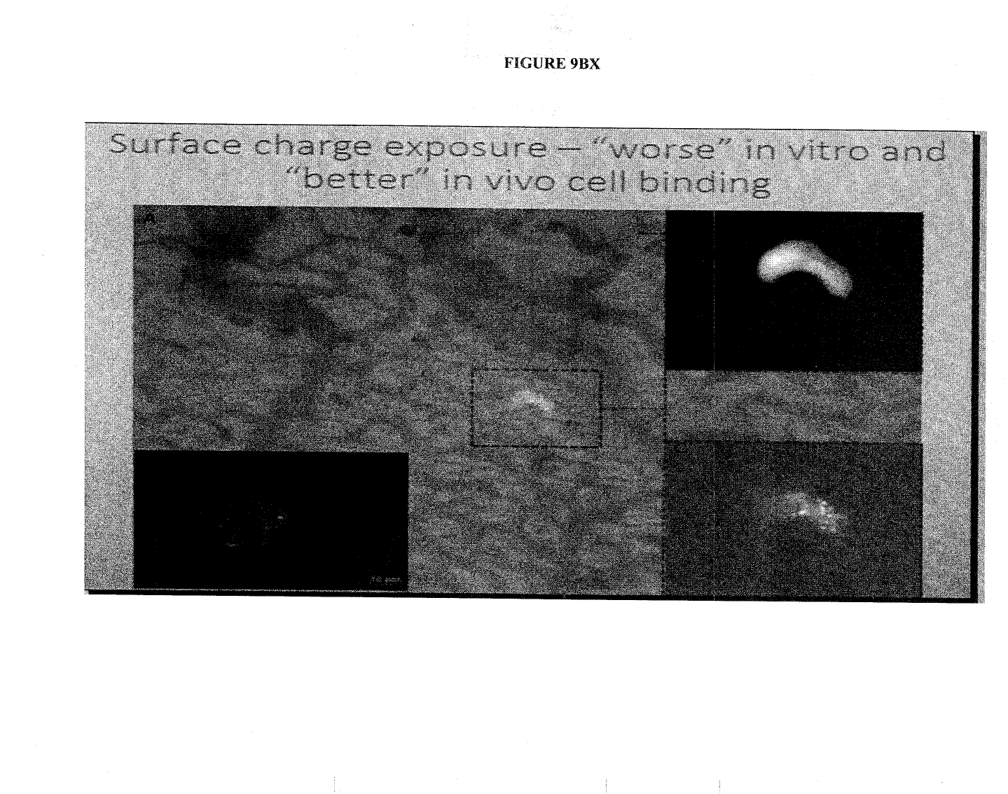

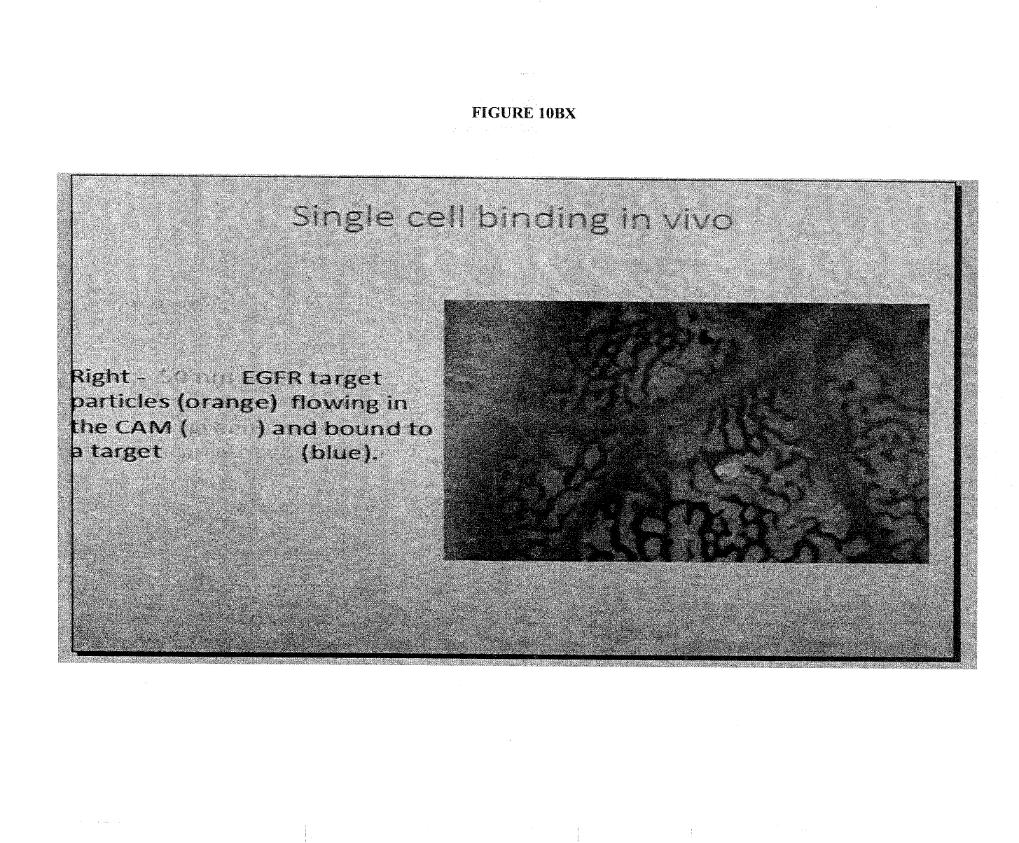

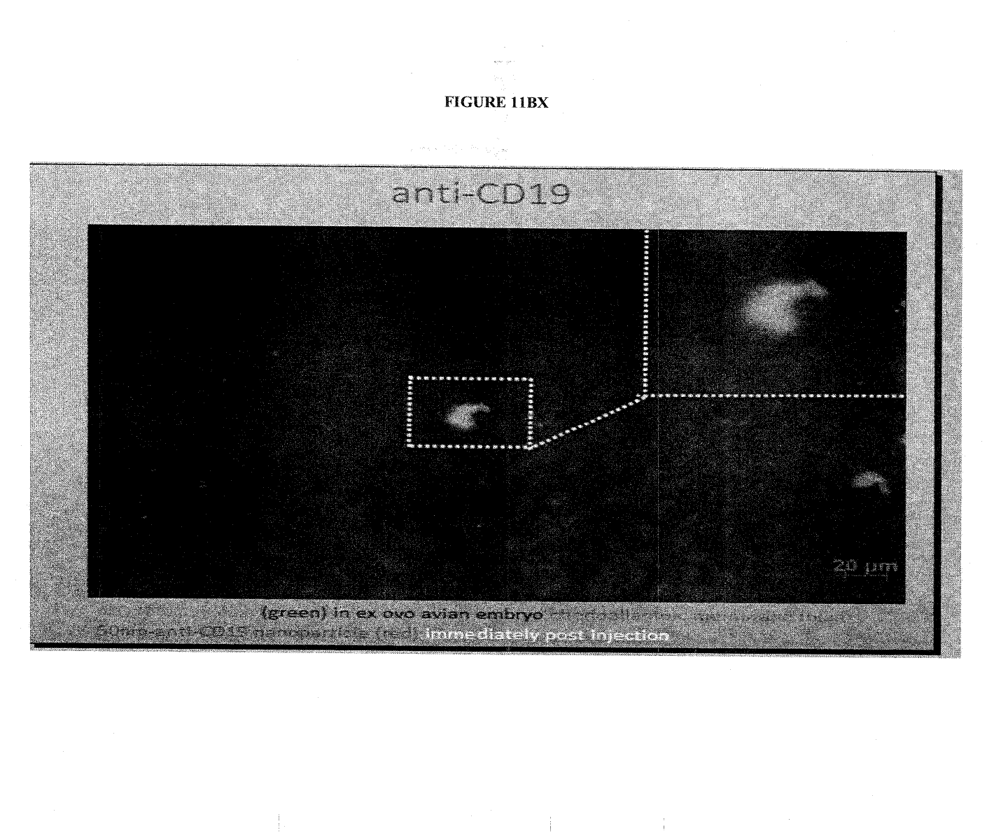

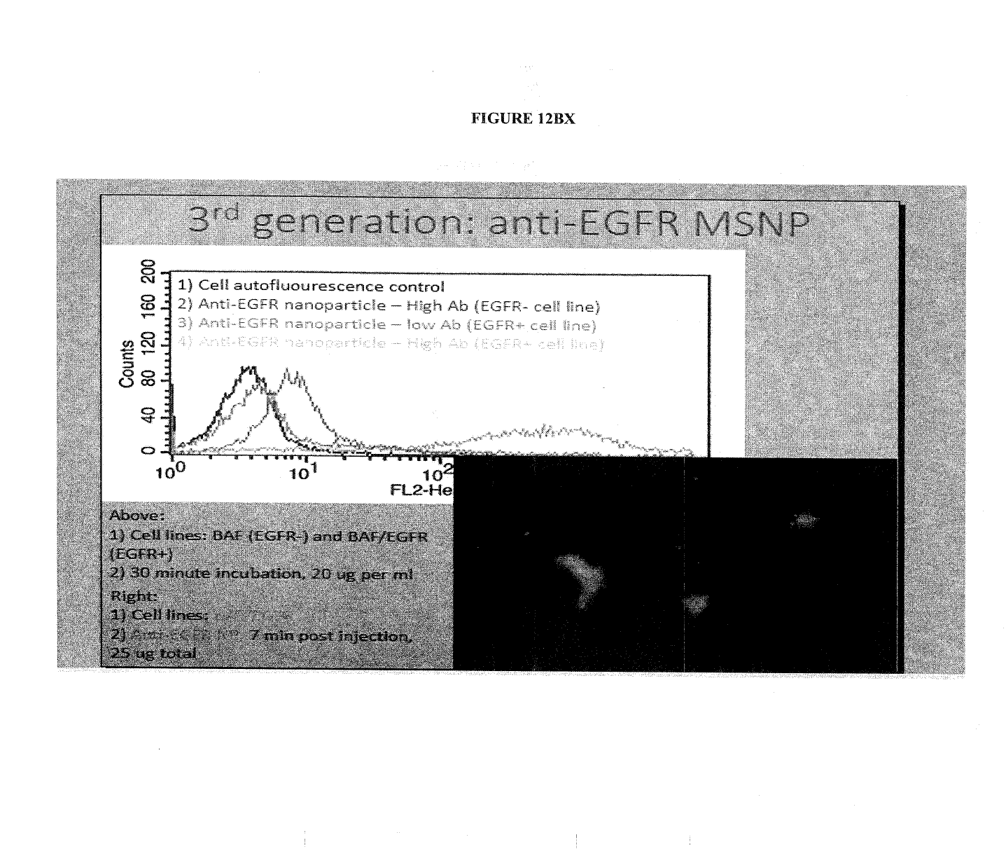

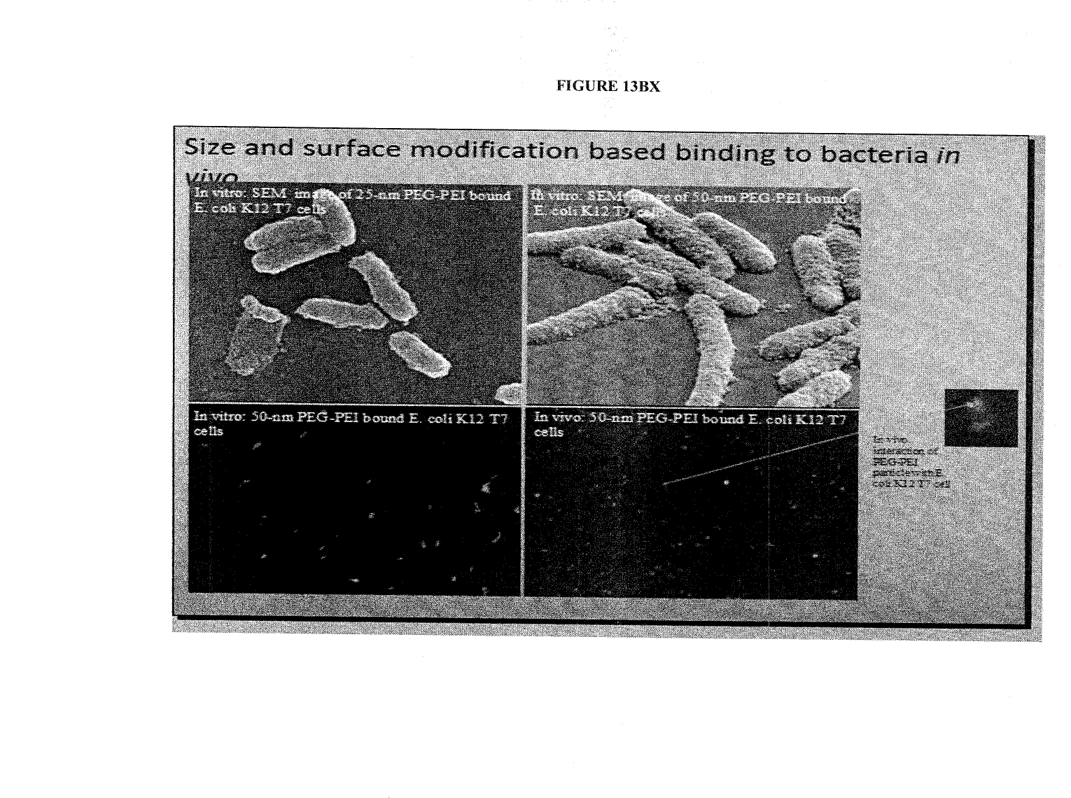

FIG. 9BX depicts specific targeting with a targeted particle modified from the synthesis described in Example 3. As determined in the experiment(s) of Example 4.

FIG. 10BX depicts single cell binding in vivo and shows an observation of the flow of nanoparticles, as well as interactions with both host and xenograft cells. As determined in the experiment(s) of Example 4.

FIG. 11BX illustrates CD19 mediated targeting of leukemia cells in vivo. As determined in the experiment(s) of Example 4.

FIG. 12BX shows particle binding specificity in vitro and in vivo (lower right). As determined in the experiment(s) of Example 4.

FIG. 13BX illustrates that PEG-PEI particles described in Example 3 can also be tuned to binding bacteria in vitro and in vivo. Binding and surface distribution on bacteria is also modified by size. As determined in the experiment(s) of Example 4.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers) or racemic mixtures, pharmaceutically acceptable salts (including alternative pharmaceutically acceptable salts when a pharmaceutically acceptable salt is disclosed) and prodrug forms. The term compound herein refers to stable compounds. Within its use in context, the term compound may refer to a single compound or a mixture of compounds as otherwise described herein. One or more bioactive agent (any agent which produces an intended biological, including pharmacological effect) may be included in MSNPs according to the present invention to provide pharmaceutical compositions hereunder.

The term “mesoporous silica nanoparticles” (MSNPs) is used to describe nanoparticles according to the present invention which are modified to target specific cells (in many instances, cancer cells) in vivo for diagnostic and/or therapeutic purposes.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. In certain preferred aspects of the present invention, where administration via intravenous, intramuscular, intraperitoneal, retro-orbital and subcutaneous injection routes produces long residence times (on the order of at least 12 hours to 2 weeks or more) and greater biodistribution and/or bioavailability, the MSNPs and protocells are monodisperse and generally no greater than about 50 nm in average diameter, often less than about 30 nm in average diameter, as otherwise described herein. The term “D₅₀” refers to the particle size below which 50% of the particles in a multiparticulate fall. Similarly, the term “D₉₀” refers to the particle size below which 90% of the particles in a multiparticulate fall.

The MSNP size distribution, according to the present invention, depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, mesoporous silica nanoparticles can range, e.g., from around 1 nm to around 500 nm in size, including all integers and ranges there between. The size is measured as the longest axis of the particle. In various embodiments, the particles are from around 5 nm to around 500 nm and from around 10 nm to around 100 nm in size. The mesoporous silica nanoparticles have a porous structure. The pores can be from around 0.5 nm to about 25 nm in diameter, often about 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, preferred MSNPs according to the present invention: are monodisperse and range in size from about 25 nm to about 300 nm; exhibit stability (colloidal stability); have single cell binding specification to the substantial exclusion of non-targeted cells; are neutral or cationic for specific targeting (preferably cationic); are optionally modified with agents such as PEI, NMe3+, dye, crosslinker, ligands (ligands provide neutral charge); and optionally, are used in combination with a cargo to be delivered to a targeted cell.

In certain alternative embodiments, the MSNPs are monodisperse and range in size from about 25 nm to about 300 nm. The sizes used preferably include 50 nm (+/−10 nm) and 150 nm (+/−15 nm), within a narrow monodisperse range, but may be more narrow in range. A broad range of particles is not used because such a population is difficult to control and to target specifically.

In certain alternative embodiments, the present invention are directed to MSNPs and preferably, protocells of a particular size (diameter) ranging from about 0.5 to about 30 nm, about 1 nm to about 30 nm, often about 5 nm to about 25 nm (preferably, less than about 25 nm), often about 10 to about 20 nm, for administration via intravenous, intramuscular, intraperitoneal, retro-orbital and subcutaneous injection routes. These MSNPs and/or protocells are often monodisperse and provide colloidally stable compositions. These compositions can be used to target tissues in a patient or subject because of enhanced biodistribution/bioavailability of these compositions, and optionally, specific cells, with a wide variety of therapeutic and/or diagnostic agents which exhibit varying release rates at the site of activity. As noted in the present application, MSNPs and protocells may have a charged surface (zeto potential) which ranges from about −40 EV to +40 EV. MSNPs and protocells according to the present invention may exhibit varied surface charges as a consequence of the componentry used to create the MSNPs. A typical MSNP based upon silica (without amine modification) exhibits a negatively charged surface having a zeta potential often within the range of about −10 EV to about −40 EV. A negative surface charge, or alternatively, a positive surface charge which is presented through use of quaternary amines for MSNPs and protocells pursuant to the present invention are consistent with these particles being less interactive with vascular/endothelial tissue and providing greater distribution to and high residence times in tissue after administration. A positively charged MSNP exhibits a zeta potential of about +10 EV to about +40 EV. A positive surface charge for MSNPs and protocells pursuant to the present invention, especially from primary amines, and to a less extent secondary and tertiary amines, are consistent with these particles being more interactive with vascular tissue and providing limited distribution principally to vascular tissue after administration. It is noted that when secondary and tertiary amines, as opposed to primary amines, are used to provide a more positively charged surface, whether the actual surface charge is negative or positive, these may exhibit non-specific binding to vascular tissue (endothelial tissue), but the effect is substantially less (muted) than the effect is for primary amines. Of course, using mixtures of amines may be used to influence both the surface charge (zeta potential) as well as the non-specific binding of the nanoparticles to vascular tissue along a continuum from very little, if any binding (quaternary amines) to some binding (tertiary and secondary amines) to specific targeting of endothelial cells utilizing primary amines.

The term “uniform surface” is used to describe a surface which contains a uniform surface charge. Uniform surfaces occur for MSNPs (preferably PEgylated) which contain quaternary amines such as the charge is consistently projected on the whole surface of the MSNP without appreciable patches or gaps in the surface charge. A “non-uniform surface” describes a surface of an MSNP which contains patches of charge which are distinguishable from the broader portions of the surface. In the case of MSNPs which are modified with primary amines, the overall surface may be neutral or charged, but the primary amine creates a patch of more positive charge with protruding protonated amines characterizing the patches on the surface of the MSNPs. The surface of the MSNPs, including protocells according to the present invention may be measured and/or identified using cryo-TEM and TEM analysis, among others. These analyses look at the characteristics of the binding of a metal with high electron density—often a heavy metal such as gold, silver, iron and the like- to produce a 3-dimensional spatial arrangement on the nanoparticle. Uniform surfaces tend to be consistent and uniform in their surface charge, whereas non-uniform surfaces tend to have areas of concentrated charge in a patchwork that can often be random.

The term “PEGylated” in its principal use refers to an MSNP which has been produced using PEG-containing silanes or zwitterionic group-containing silanes to form the MSNP. In general, the amount of the PEG-containing silanes and/or zwitterionic-containing silanes which optionally are used to produce MSNPs according to the present invention represent about 0.05% to about 50% (about 0.1% to about 35%, about 0.5% to about 25%, about 1% to about 20%, about 2.5% to about 30%, about 0.25% to about 10%, about 0.75% to about 15%) by weight of these monomers in combination with the silane monomers which are typically used to form MSNPs. A PEG-containing silane is any silane which contains a PEG as one of the substituents and the remaining groups can facilitate the silane reacting with other silanes to produce MSNPs according to the present invention. Preferred PEG-containing silanes and/or zwitterionic-containing silanes which are used in the present invention to create PEGylated MSNPs include 2-[methoxy(polyethyleneoxy)propyl]trimethoxysilane (containing varying molecular weights of PEG ranging from about 100 to 10,000 average molecule weight, often about 200 to 5,000 average molecular weight, about 1,000-2,500 average molecular weight, about 1500-2000 average molecular weight) and 3-{[Dimethoxyl(3-trimethoxysilyl)propyl]ammonio)propane-1-sulfonate and mixtures thereof, among others. The term “PEGylated” may also refer to lipid bilayers which contain a portion of lipids which are PEGylated (from about 0.02% up to about 50%, about 0.1% to about 35%, about 0.5% to about 25%, about 1% to about 15%, about 0.5% to about 7.5%, about 1% to about 12.5% by weight of the lipids used to form the lipid bilayer or multilayer). These lipids often are amine-containing lipids (e.g DOPE and DPPE) which are conjugated or derivatized to contain a PEG group (having an average molecule weight ranging from about 100 to 10,000, about 200 to 5,000, about 1,000-5,000, including 1,000, 2000, 3000 and 3400) and combined with other lipids to form the bilayer/multilayer which encapsulates the MSNP.

The term “non-specific binding” refers to the binding which occurs between a charged surface of the MSNPs according to the present invention and endothelial tissue pursuant to the present invention because the interaction between the particles and the tissue surface are based non-specifically upon the interactions of charges on the particles and the tissue surface rather than a ligand-ligand interaction. In the case of “specific binding” the interaction between the particle and a target is based upon a specific ligand-ligand interaction. It is noted that when a particle exhibits low non-specific binding, that particle may exhibit very little binding (i.e. little specific or non-specific binding) or more specific binding (greater ligand-ligand interaction) depending upon the context of its use.

The terms “targeting ligand” and “targeting active species” are used to describe a compound or moiety (preferably an antigen) which is complexed or preferably covalently bonded to the surface of a MSNPs and/or protocells according to the present invention which binds to a moiety on the surface of a cell to be targeted so that the MSNPs and/or protocells may selectively bind to the surface of the targeted cell and deposit their contents into the cell. The targeting active species for use in the present invention is preferably a targeting peptide as otherwise described herein, a polypeptide including an antibody or antibody fragment, an aptamer, or a carbohydrate, among other species which bind to a targeted cell.

Preferred ligands which may be used to target cells include peptides, affibodies and antibodies (including monoclonal and/or polyclonal antibodies). In certain embodiments, targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells). Other targeting peptides known in the art may also be used.

The charge of the nanoparticle is controlled based on what is to be accomplished (via PEI, NMe3+, dye, crosslinker, ligands, etc.), but for targeting vascular tissue the charge is preferably cationic. In the case of enhanced biodistribution, the charge is preferably anionic, but may be cationic provided that the charge occurs principally from the inclusion of quaternary amines. Charge also changes throughout the process of formation. Initially, in certain embodiments the targeted particles are cationic and are often delivered as cationically charged nanoparticles, however post modification with ligands they are closer to neutral. The ligands which find use in the present invention include peptides, affibodies and antibodies, among others. These ligands are site specific and are useful for targeting specific cells which express peptides to which the ligand may bind selectively to targeted cells.

MSNPs pursuant to the present invention may be used to deliver cargo to a targeted cell, including, for example, cargo component selected from the group consisting of a polynucleotide such as DNA, including double stranded linear DNA or a plasmid DNA, RNA, including small interfering RNA, small hairpin RNA, microRNA, a drug (in particular, an anticancer drug such as a chemotherapeutic agent), an imaging agent, or a mixture thereof.

In protocells of the invention, a PEGylated lipid bi- or multilayer encapsulates a population of MSNPs as described herein and comprises (1) an optionally-thiolated PEG (2) at least one lipid and, optionally (3) at least one targeting ligand which is conjugated to the outer surface of the lipid bi- or multilayer and which is specific against one or more receptors of white blood cells and arterial, venous and/or capillary vessels or combinations thereof, or which is specific against one or more receptors of targets a cancer cell, a bacterium, or a virus.

Protocells of the invention are highly flexible and modular. High concentrations of physiochemically-disparate molecules can be loaded into the protocells and their therapeutic and/or diagnostic agent release rates can be optimized without altering the protocell's size, size distribution, stability, or synthesis strategy. Properties of the supported lipid bi- or multilayer and mesoporous silica nanoparticle core can also be modulated independently, thereby optimizing properties as surface charge, colloidal stability, and targeting specificity independently from overall size, type of cargo(s), loading capacity, and release rate.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, delay in or inhibition of the likelihood of the onset of the disease, etc. In the case of viral infections, these terms also apply to viral infections and preferably include, in certain particularly favorable embodiments the eradication or elimination (as provided by limits of diagnostics) of the virus which is the causative agent of the infection. Treatment can also be used to provide prevention (prophylaxis/reducing the likelihood) of a disease state occurring, but the present invention contemplates a distinction between the treatment of a disease state and/or condition and the prevention (prophylaxis/reducing the likelihood) that a disease state or condition will occur, within the context of such treatment/prevention.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

Treatment, as used herein, may encompass prophylactic and/or therapeutic treatment depending on context, principally of cancer, but also of other disease states, including bacterial and viral infections, (e.g. HIV, HBV and/or HCV) and the treatment of inflammation of the lungs, kidneys and pancreas as well as numerous vascular disorders. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present invention is effective to decrease the severity of the disease or lengthen the lifespan of the mammal so afflicted, as in the case of cancer, or inhibit or even eliminate the causative agent of the disease, as in the case of human immunodeficiency virus (HIV I or II), hepatitis B virus (HBV) and/or hepatitis C virus infections (HCV) infections.

Our novel MSNPs and protocells can also be used to treat a wide variety of bacterial infections including, but not limited to, infections caused by bacteria selected from the group consisting of F. tularensis, B. pseudomallei, Mycobacterium, staphylococcus, streptococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae and P. aeruginosa.

Antibiotic MSNPs and protocells of the invention can contain one or more antibiotics, e.g. “Antibiotics” include, but are not limited to, compositions selected from the group consisting of Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazadime, Ceftibuten, Ceftizoxime Ceftriaxone, Cefepime, Ceftaroline fosamil, Ceftobiprole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidonones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demeclocycline, Doxycycline, Vibramycin Minocycline, Tigecycline, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

The term “anticancer agent” or “additional anticancer agent” (depending on the context of its use) shall mean chemotherapeutic agents such as an agent selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

MSNPs and protocells of the invention also can comprise anti-cancer agents selected from the group consisting of doxorubicin-loaded liposomes that are functionalized by polyethylene glycol (PEG), antimetabolites, inhibitors of topoisomerase I and II, alkylating agents and microtubule inhibitors, adriamycin aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof.

In certain embodiments, MSNPs and protocells of the invention comprise anti-cancer drugs selected from the group consisting of doxorubicin, melphalan, bevacizumab, dactinomycin, cyclophosphamide, doxorubicin liposomal, amifostine, etoposide, gemcitabine, altretamine, topotecan, cyclophosphamide, paditaxel, carboplatin, cisplatin, and taxol.

MSNPs and protocells of the invention can include one or more antiviral agents to treat viral infections, especially including HIV infections, HBV infections and/or HCV infections. Exemplary anti-HIV agents include, for example, nucleoside reverse transcriptase inhibitors (NRTI), non-nucloeoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCHSO3034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

The above compounds/bioactive agents may also be charged to MSNPs, preferably including protocells, having average diameters which are less than about 50 nm, more preferably less than 30 nm for formulating compositions adapted for intravenous, intramuscular, intraperitoneal, retro-orbital and subcutaneous injection routes. In certain embodiments, subcutaneous routes of administration are preferred for administering bioactive agents including vaccines (immunogenic compositions), opiates, hormones (epinephrine, growth hormone, insulin, etc.), synthetic steroidal agents, fertility agents, contraceptive agents and antibodies, especially including therapeutic monoclonal antibodies. Additional specific bioactive agents which may be administered favorably by subcutaneous routes according to the present invention include, for example, alfentanil, cyclizine, dexamethasone, diamorphine, glycopyrronium, haloperidol, hydromorphine, hyoscine butylbromide, hyoscine hydrobromide, ketamine, keterolac, levomepromazine, metoclopramide, midazolam, morphine (morphine sulfate), octreotide, oxycodone and pharmaceutically acceptable salts and/or alternative salts.

MSNPs and protocells of the invention can also be used to diagnose and treat a “vascular disorder”. A “vascular disorder” includes but is not limited to ischemic stroke, hemorrhagic stroke, transient ischemic attack (TIA), vascular inflammation due to meningitis, atherosclerosis, thrombi or emboli resulting from atherosclerosis, arteritis, physical obstruction of arterial blood supply to the brain, lacunar stroke, hypoperfusion emboding diffuse injury caused by non-localized cerebral ischemia, myocardial infarction and arrhythmia, restenosis associated with percutaneous transluminal coronary angioplasty, peripheral vascular disease and cerebral vascular disease, venous occlusive disorders such as deep vein thrombosis, and hypercoagulopathies. Vascular disease treatments include but are not limited to treatment of peripheral artery diseases (e.g. with cholesterol-lowering medications, high blood pressure medications, medication to control blood sugar, medications to prevent blood clots, symptom-relief medications, angioplasty and surgery, thrombolytic therapy and supervised exercise programs), cerebrovascular disorder treatments (e.g. aspirin, TPA, mechanical clot removal, carotid endarterectomy, angioplasty and stents), treatment of atherosclerosis (e.g. cholesterol medications, anti-platelet medications, beta blocker medications, angiotensin-converting enzyme (ACE) inhibitors, calcium channel blockers, water pills (diuretics), angioplasty, endarterectomy, thrombolytic therapy, and bypass surgery).

Compositions according to the present invention may be used to treat a large number of vascular disease states and conditions including one or more of aggressive angiomyxoma, anemic infarct, aneurysm, angiopathy, annuloaortic ectasia, aortitis, aortoiliac occlusive disease, arterial stiffness, arteriosclerosis, arteriolosclerosis, atheroma, atherosclerosis, brain ischemia, thomboangitis obliterans, capillaritis, carotid arter dissection, carotid artery stenosis, carotid bruit, cerebral amyloid angiopathy, cholesterol embolism, chronic cerebrospinal venous insufficiency, circulating endothelial cell, collapsed being, corona phlebectatica, degos disease, diabetic angiopathy, diabetic nephropathy, dural arteriovenous fistula, endothelial dysfunction, endotheliitis, familial aortic dissection, fatty streak, fibromuscular dysplasia, hemorrhagi infarct, hereditary cystatin C amyloid angiopathy, Hollenhorst plaque, hot aches, hyaline arteriosclerosis, hyperplastic arteriolosclerosis, hypotension, inferior vena cava syndrome, intermitten claudication, intradural pseudoaneurysm, jugular vein ecstasia, Kawasaki disease, Loeys-Dietz syndrome, macrovascular disease, metanephric dysplatic hematoma of the sacral region, Monckeberg's arteriosclerosis, oblitering endarteritis, orthostatic hypertension, orthostatic hypotension, orthostatic intolerance, Paget-Schroetter disease, paradoxical embolism, peripheral vasculopathy, phlebitis, phlegmasia alba dolens, phlegmasia cerulea dolens, portocaval anastomosis, portal veing thrombosis, postural orthostatic tachycardia syndrome, pseudothrombophlebitis, pulmonary artery sling, pulmonic regurgitation, pylephlebitis, Rasmussen's aneurysm, Sack-Barabas syndrome, Stewart-Treves syndrome, superiod mesenteric artery syndrome, Susac's syndrome, Takyasu's arteritis, thoracic aortic aneurysm, thrombophlebitis, varicocele, vascular anomaly, vascular disease, vascular malformation, vascular malformation, vaso-occlusive crisis, vegetative-vascular dystonia, venous ulcer, vertebral artery dissection and vertobrovasilar insufficiency, among others.

Compositions according to the present invention may also be used to treat inflammation in the pancreas, lungs and kidneys. These inflammatory disease states and/or conditions include, for example inflammatory diseases (inflammation) of the pancreas, lungs and kidneys caused by any etiology, and include acute and chronic pancreatitis, lung inflammation such as obstructive lung disease (COPD), emphysema, pleurisy, asthma, wet lung inflammation, etc., kidney inflammation such as glomerulonephritis, membrane proliferative glomerulonephritis, Berger's disease, pyelonephritis, lupus nephritis, kidney inflammation related to CKD, Goodpasture's syndrome, Wegener's granulomatosis.

In certain embodiments, the MSNPs and protocells according to the present invention contain an agent for treating peripheral vascular disease as cargo. Such agents include abciximab, alprostadil, prostin, statins, including cerivastatin, simvastatin, clopidrogel, colesevelam, cilostazol, ezetimibe, isosorbide, triclopidine and warfarin, among others. These agents may be targeted to the vasculature of a patient using MSNPs and protocells having a cationic charge.

Typically the MSNPs and protocells according to the present invention are loaded with cargo to a capacity up to about 50 weight % or more (from about 0.01% to about 50%, about 0.02% to about 40%, about 0.2 to about 35%, about 0.5% to about 25%, about 1% to about 25%, about 1.5% to about 15%, about 0.1% to about 10%, about 0.01% to about 5%): defined as (cargo weight/weight of loaded protocell)×100. The optimal loading of cargo is often about 0.01 to 10% but this depends on the drug or drug combination which is incorporated as cargo into the MSNPs. This is generally expressed in μM per 10¹⁰ particles where we have values ranging from 2000-100 μM per 10¹⁰ particles. Preferred MSNPs according to the present invention exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the MSNPs according to one embodiment of the present invention, the surface area is mainly internal as opposed to the external geometric surface area of the nanoparticle.

The term “lipid” is used to describe the components which are used to form lipid bi- or multilayers on the surface of the nanoparticles which are used in the present invention. Various embodiments provide nanostructures which are constructed from nanoparticles which support a lipid bilayer(s). In embodiments according to the present invention, the nanostructures preferably include, for example, a core-shell structure including a porous particle core surrounded by a shell of lipid bilayer(s). The nanostructure, preferably a porous alum nanostructure as described above, supports the lipid bilayer membrane structure.

The lipid bi- or multilayer supported on the porous particle according to one embodiment of the present invention has a lower melting transition temperature, i.e. is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bi- or multilayer may vary significantly in composition. Ordinarily, any lipid or polymer which may be used in liposomes may also be used in MSNPs according to the present invention. Preferred lipids are as otherwise described herein.

In embodiments according to the invention, the lipid bi- or multilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocells and/or a targeted delivery into a cell to maximize an immunogenic response. PEG, when included in lipid bilayers (using PEGylated lipids), can vary widely in molecular weight (although PEG ranging from about 10 to about 100 units of ethylene glycol, about 15 to about 50 units, about 15 to about 20 units, about 15 to about 25 units, about 16 to about 18 units, etc, may be used) and the PEG component which is generally conjugated to a phospholipid through an amine group comprises about 1% to about 20%, preferably about 5% to about 15%, about 10% by weight of the lipids which are included in the lipid bi- or multilayer.

Numerous lipids which are used in liposome delivery systems may be used to form the lipid bi- or multilayer on nanoparticles to provide MSNPS according to the present invention. Virtually any lipid which is used to form a liposome may be used in the lipid bi- or multilayer which surrounds the nanoparticles to form MSNPS according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the present invention given the fact that cholesterol may be an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including immunogenic peptides, proteins and antibodies, RNA and DNA through the amine group on the lipid.

MSNPs and protocells of the invention can be PEGylated with a variety of polyethylene glycol-containing compositions. PEG molecules can have a variety of lengths and molecular weights and include, but are not limited to, PEG 200, PEG 1000, PEG 1500, PEG 4600, PEG 10,000, PEG-peptide conjugates or combinations thereof. Example 3 herein describes the use of 2-[methoxy(polyethyleneoxy)-propyl]trimethoxysilane (MW 550-750, 9-12 EO, PEG-silane) for MSNP PEGylation. Typically, pegylation occurs by using a silyl agent containing a PEG groups (PEG-silane) which is added to the silane mixture in synthesizing MSNPs according to the present invention. Alternatively, a reactive amine group on the surface of the MSNPs may be functionalized by reacting the amine with a PEG containing group to form a PEG group on the amine.

The term “reporter” is used to describe an imaging agent or moiety which is incorporated into the phospholipid bilayer or cargo of MANPS according to an embodiment of the present invention and provides a signal which can be measured. The moiety may provide a fluorescent signal or may be a radioisotope which allows radiation detection, among others. Exemplary fluorescent labels for use in MSNPs and protocells (preferably via conjugation or adsorption to the lipid bi- or multilayer or silica core, although these labels may also be incorporated into cargo elements such as DNA, RNA, polypeptides and small molecules which are delivered to cells by the protocells) include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art.

Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

Pharmaceutical compositions according to the present invention comprise an effective population of MSNPs and/or protocells as otherwise described herein formulated to effect an intended result (e.g. immunogenic result, therapeutic result and/or diagnostic analysis, including the monitoring of therapy) formulated in combination with a pharmaceutically acceptable carrier, additive or excipient. The MSNPs and/or protocells within the population of the composition may be the same or different depending upon the desired result to be obtained. Pharmaceutical compositions according to the present invention may also comprise an addition bioactive agent or drug, such as an antibiotic or antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when different populations of MSNPs and/or protocells are used in the pharmaceutical compositions or when additional bioactive agent(s) are used in combination with one or more populations of protocells as otherwise described herein.

In certain preferred formulation embodiments of the invention include protocells comprised of mesoporous silica nanoparticulates (MSNPs) that (a) are loaded with one or more pharmaceutically-active agents and (b) that are encapsulated by and that support a lipid bilayer, and wherein the protocell has an average diameter of between about 1 nm to about 50 nm, more preferably between about 1 nm to about 30 nm, about 5 nm to about 25 nm, often 10 nm to about 25 nm, about 10 to about 20 nm. It has unexpectedly been discovered that the administration of protocells comprising therapeutic and/or diagnostic agents via intravenous, intramuscular, intraperitoneal, retro-orbital and especially subcutaneous routes of administration at the average diameters indicated above provide enhanced biodistribution, enhanced bioavailability and increased residence time (often at least 12-24 hours to several days up to a week or in certain cases, two weeks to a month or even longer), of these protocells compared to protocells with average diameters which are in excess of 50 nm, often greater than about 100 nm or more (e.g. 200-300 nm). Accordingly, the present compositions and methods of treatment and diagnosis in these routes of administration are greatly facilitated compared to compositions which contain protocells of larger diameters. Compositions according to the present invention may be used to administer cargo as otherwise described herein to a patient or subject through intravenous, intramuscular, intraperitoneal, retro-orbital and subcutaneous routes of administration, with unexpected biodistribution, bioavailability and residence times far exceeding compositions utilizing nanoparticles with average diameters in excess of 50-100 nm or greater (200-250 nm).

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of MSNPs and/or protoells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present invention.

Methods of treating patients or subjects in need for a particular disease state or infection comprise administration an effective amount of a pharmaceutical composition comprising therapeutic MSNPs and/or protocells and optionally at least one additional bioactive (e.g. antiviral) agent according to the present invention.

Diagnostic methods according to the present invention comprise administering to a patient in need an effective amount of a population of diagnostic MSNPs and/or protocells (e.g., MSNPs and/or protocells which comprise a target species, such as a targeting peptide which binds selectively to cancer cells and a reporter component to indicate the binding of the protocells whereupon the binding of protocells to cells as evidenced by the reporter component (moiety) will enable a diagnosis of the existence of a disease state in the patient.

An alternative of the diagnostic method of the present invention can be used to monitor the therapy of a disease state in a patient, the method comprising administering an effective population of diagnostic MSNPs and/or protocells (e.g., MSNPs and/or protocells which comprise a target species, such as a targeting peptide which binds selectively to target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

The present invention also is directed to a process or processes for preparing the MSNPs according to the present invention.

In one embodiment, the invention is directed to a process for making a population of optionally PEGylated, monodisperse mesoporous silica nanoparticles (MSNPs) that exhibit a relatively non-uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60, 55, 50, 45, 40, 35 or 30 nm (more preferably, less than 50 nm, even more preferably, less than 30, 25, 20, 15 or 10 nm)), a pore size of between about 1 nm to about 200 nm or between about 50, 40, 30, 25, 20, 15, 10, 9, 8, 7, 6, 5, 4, 3, 2 or 1 nm, a surface area of between about 100-1,000 m²/g, and a Zeta potential (ζ) of between about −40 mV to about +40 mV (more preferably greater than 0 mV) and that, upon administration in vivo, exhibit non-specific binding to white blood cells and arterial, venous and/or capillary vessels or combinations thereof, the process comprising:

(a) (1) preparing a mesoporous silica colloidal solution comprising:
(1) a solvent solution comprising:
(i) an alkoxysilane selected from the group consisting of tetramethylortho silicate (TMOS), tetraethylortho silicate (TEOS), tetrakis(2-hydroxyethyl)ortho silicate (THEOS), methyldiethoxysilane (MDES), 3-(glycidoxypropyl)triethoxysilane (GPTMS), 3-(trimethyoxysilyl)propylacrylate (TMSPA), N-(3-triethoxysilylpropyl)pynole (TESPP), vinyltriethyoxysilane (VTES), methacryloxypropyltriethoxysilane (TESPM), diglycerylsilane (DGS), methyltriethoxysilane (MTMOS), trimethylmethoxysilane (TMMS), ethyltriethoxysilane (TEES), n-propyltriethoxysilane (TEPS), n-butyltriethyoxysilane (TEBS), 3-aminopropyltriethoxysilane (APTS), 2-(2,4-dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), and triethoxysilyl-terminated poly(oxypropylene)
(ii) a solvent
(iii) optionally, a reporter, and
(2) a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethylammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., most preferably at about 50° C.;
(b) including in said mesoporous silica colloidal solution a composition comprising a primary amine group and, optionally, a PEG-silane compound to produce a nanoparticle containing amine groups; and
(c) hydrothermally treating the aminated nanoparticles produced in step (b) by heating the nanoparticles at a temperature of between about 100° C. to about 150° C. (more preferably about between about 110° C. to about 140° C., still more preferably between about 115° C. to about 135° C., still more preferably at about 120° C.) to yield the optionally PEGylated, monodisperse mesoporous silica nanoparticles (MSNPs);
wherein the process can be one pot or in steps.

In the above process, (a) the alkoxysilane is preferably 3-aminopropyltriethoxysilane (APTS), the solvent is preferably N, N-dimethyl formamide (DMF) and the reporter is preferably rhodamine B isothiocynate (RITC); and the composition comprising a primary amine group is trimethoxysilylpropyl modified polyethyleneimine (50% in isopropanol, M.W. 1500-1800, PEI-silane) and the PEG-silane compound is methoxy(polyethyleneoxy)propyl]trimethoxysilane (Mw 550-750, 9-12 EO, PEG-silane).

In an alternative embodiment, the present invention is directed to a process for making a population of optionally PEGylated, monodisperse mesoporous silica nanoparticles (MSNPs) that exhibit a relatively uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60, 55, 50, 45, 40, 35 or 30 nm (more preferably, less than 50 nm, even more preferably, less than 30, 25, 20, 15 or 10 nm)), a pore size of between about 1 nm to about 200 nm or between about 50, 40, 30, 25, 20, 15, 10, 9, 8, 7, 6, 5, 4, 3, 2 or 1 nm, a surface area of between about 100-1,000 m²/g, and a Zeta potential (ζ) of between about −40 mV to about +40 mV (more preferably less than 0 mV) and that, upon administration in vivo, exhibit minimal non-specific binding and prolonged circulation, the process comprising:

(a) (1) preparing a mesoporous silica colloidal solution comprising:
(1) a solvent solution comprising:
(i) an alkoxysilane selected from the group consisting of tetramethylortho silicate (TMOS), tetraethylortho silicate (TEOS), tetrakis(2-hydroxyethyl)ortho silicate (THEOS), methyldiethoxysilane (MDES), 3-(glycidoxypropyl)triethoxysilane (GPTMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycerylsilane (DGS), methyltriethoxysilane (MTMOS), trimethylmethoxysilane (TMMS), ethyltriethoxysilane (TEES), n-propyltriethoxysilane (TEPS), n-butyltriethyoxysilane (TEBS), 3-aminopropyltriethoxysilane (APTS), dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), and triethoxysilyl-terminated poly(oxypropylene)
(ii) a solvent
(iii) optionally, a reporter, and
(2) a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethylammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., most preferably at about 50° C.;
(b) including in the mesoporous silica colloidal solution (i) a composition that does not comprise a primary amine group and, optionally, (ii) a PEG-silane compound to produce a nanoparticle containing amine groups (preferably quaternary, but also secondary and/or tertiary amine) which are not primary amine groups; and
(c) hydrothermally treating the nanoparticles produced in step (b) by heating the nanoparticles at a temperature of between about 100° C. to about 150° C. (more preferably about between about 110° C. to about 140° C., still more preferably between about 115° C. to about 135° C., still more preferably at about 120° C.) to yield the optionally PEGylated, monodisperse mesoporous silica nanoparticles (MSNPs);
wherein the process can be one pot or in steps.

In the above process, (a) the alkoxysilane is 3-aminopropyltriethoxysilane (APTS), the solvent is N, N-dimethyl formamide (DMF) and the reporter is rhodamine B isothiocynate (RITC); and (b) the composition that does not comprise a primary amine group is N-trimethoxysilylpropyl-N,N,N-trimethyl ammonium chloride (50% in methanol, TMAC-silane) and the PEG-silane compound is methoxy(polyethyleneoxy)propyl]trimethoxysilane (Mw 550-750, 9-12 EO, PEG-silane).

Additional embodiments are directed to MSNPs and/or populations of MSNPs which are produced by the above methods.

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL.

The invention is described further in the following non-limiting examples.

### Example 1

### Synthesis of MSNPs According to the Present Invention

To prepare dye-labeled PEG/PEI MSNPs at a single batch/unit level, 3 mg of RITC (or suitable NHS-Ester terminated dyes) is first dissolved in 2 mL of DMF and 1.5 μL of APTES is then added and the stirred at room temperature for 4 h to prepare the RITC-APTES solution. Then, 150 mL of 2.6-7.9 mM CTAB 0.1-1.5 ammonium hydroxide solution was prepared in a beaker, sealed, and placed in an oil bath at 30-60° C. After continuously stirring for 1 h, 2.5 mL of dilute TEOS solution (prepared in ethanol) was added to the surfactant solution. After another hour stirring, 225-450 μL of PEG-silane and 2.5-400 μL of PEI-silane were added consecutively to the mesoporous silica colloidal solution under stirring. Thirty minutes later, stirring was stopped and the as-synthesized dye-PEG/PEI colloidal solution was aged at 30-60° C. for 10-20 h. After aging, the obtained particle solution (˜50 mL) underwent a hydrothermal treatment (60-120° C. for 12-48 h). Following surfactant removal step, purified MSNPs were either functionalized with an NHS-Ester/PEG/Maleimide (NPM) crosslinker or modified with Trauts reagent. Nanoparticles modified with NPM crosslinker were incubated for at least 30 min at room temperature prior to washing in water. Surface functionalization of NPM particles is completed by addition of cysteine terminated peptides, affibodies or antibodies, although this crosslinking chemistry can be replaced with other generic strategies as required. For particles treated with Trauts reagent, particles are incubated in the reagent for at least 30 min prior to centrifugation and washing with water. Nanoparticles are then modified with a maleimide-PEG-avidin (MPA) crosslinker by incubation for at least 30 min. After centrifugation and washing, MPA functionalized MSNPs can be modified with any targeting moiety to which biotion has been conjugated (antibodies, affibodies and peptides to date). Following at least 5 minutes of incubation followed by washing, particles are ready for use. In addition to 50 nm PEG-PEI MSNPs, this procedure can also be applied to particles between 25 and 300 nm.

FIG. 1 shows the importance of colloidal stability for nanotherapeutics made by syntheses such as that set forth above. FIG. 2 shows the high resolution of cellular-nanoparticle interaction imaging in vivo, and FIG. 3 indicates that targeting of ligands increases the cell specific accumulation of MSNPs in vivo. FIG. 4 shows an in vivo targeting attempt using PEI and EGFR. FIG. 5 shows a cell targeting attempt which evidences that the number or concentration of particles will influence the ability of the MDNP to target cells. FIG. 6, shows targeted particle binding. FIG. 7 shows the in vivo tumor targeting of MSNPs (PEG/NMe₃⁺) pursuant to the present invention. FIG. 8 shows SP94 targeted MSNPs in vivo.

FIG. 9 shows the binding of 50 nm CD19 Ab nanparticles with certain identified cells.

### Example 2

### Synthesis of PEG-PEI, PEG-TMS and PEG-NMe₃⁺

Synthesis of stable fluorescent dye-incorporated PEG/PEI, PEG/TMS and PEG/NMe₃⁺ MSNPs (designated as PEG-PEI, PEG-TMS and PEG-NMe₃⁺): To prepare dye-labeled PEG/PEI MSNPs at a single batch/unit level, 3 mg of RITC (or suitable NHS-Ester terminated dyes) is first dissolved in 2 mL of DMF and 1.5 μL of APTES is then added and the stirred at room temperature for 4 h to prepare the RITC-APTES solution. Then, 150 mL of 2.6-7.9 mM CTAB 0.1-1.5M ammonium hydroxide solution was prepared in a beaker, sealed, and placed in an oil bath at 30-60° C. After continuously stirring for 1 h, 2.5 mL of dilute TEOS solution (prepared in ethanol) was added to the surfactant solution. After another hour stirring, 225-450 μL of PEG-silane and 2.5-400 μL of PEI-silane were added consecutively to the mesoporous silica colloidal solution under stirring. Thirty minutes later, stirring was stopped and the as-synthesized dye-PEG/PEI colloidal solution was aged at 30-60° C. for 10-20 h. After aging, the obtained particle solution (˜50 mL) underwent a hydrothermal treatment (60-120° C. for 12-48 h). The obtained dye-labeled as-synthesized MSNPs were treated with ethanolic ammonia nitrate and ethanolic hydrochloric acid solutions to remove the CTAB surfactants. For dye-labeled PEG-NMe³⁺ MSNP synthesis, the only difference was using TMAC-silane (250 μL) instead of PEI-silane. For large 150-nm-diameter PEG-NMe₃⁺ MSNP synthesis, the concentration of NH₄OH and the amount of TMAC-silane were changed 0.5 M of NH₄OH and 300 μL of TMAC-silane, respectively. Otherwise, the synthesis procedure was similar to 50 nm PEG-PEI and PEG-NMe₃⁺ MSNPs. In addition to 50 and 150 nm PEG-PEI MSNPs, this procedure can also be applied to particles between 25 and 300 nm.

Acetylation of PEG/PEI MSNPs: For acetylation of PEG/PEI MSNPs, 2 mg of PEG/PEI MSNPs was dispersed to 1 mL of DMF by centrifugation. Then, 20 μL of acetic anhydride and 10 μL of pyridine were added to the PEG/PEI NP solution. After 30 min, the acetylated particles were centrifuged and redispersed in 1 mL of DMF. The acetylation reaction on the particles was repeated one time. The acetylated particles were washed with D.I. water 3 more times by centrifugation. Finally, the acetylated PEG-PEI MSNPs were dispersed in DI water at 2 mg/mL.

FIG. 1A shows the importance of size in altering the rate and location of deposition of MSNPs (e.g. made by syntheses such as that described in this example) in blood vessels in vivo. FIG. 2A shows that the cationic charge exposure of the MSNPs modifies the rate and location of the binding of the MSNPs. FIG. 3A shows the charge exposure of the MSNPs relative to the size of the particles. FIG. 4A shows an in vivo targeting attempt using PEI and EGFR.

### Example 3

### Re-Examining the Size/Charge Paradigm: Differing In Vivo Characteristics of Size and Charge-Matched Mesoporous Silica Nanoparticles

Here we demonstrate that two types of mesoporous silica nanoparticles (MSNP) that are matched in terms of primary and hydrodynamic particle size, shape, pore structure, colloidal stability, and zeta potential, but differ in the spatial arrangement and relative exposure of surface amines, have profoundly different interactions with cells and tissues when evaluated in vitro and in vivo. While both particles are ˜50 nm in diameter, PEGylated, and positively charged (ζ=+40 mV), PEG-PEI (MSNPs modified with exposed polyamines), but not PEG-NMe₃⁺ (MSNP modified with distributed, obstructed amines) rapidly bind serum proteins, diverse cells types in vitro, and endothelial and white blood cells in vivo (ex ovo chick embryo model). This finding helps elucidate the relative role of surface exposure of charged molecules vs zeta potential in otherwise physicochemically matched MSNP and highlights protein corona neutrality as an important design consideration when synthesizing cationic NPs for biological applications.

Nanoparticle (NP)-cell interactions, particularly in complex in vivo microenvironments, are regulated by an intricate spatiotemporal interplay of numerous biological and NP characteristics. Multiple NP physicochemical properties including, at the most basic level, material composition, size, shape, surface charge, and surface chemistry, have all been reported to play significant roles.¹⁻³ However, the relative importance of these diverse NP physicochemical properties in regulating interactions with various biological systems remains incompletely understood.¹ As such, achieving or avoiding cell-type-specific interactions in vivo requires an improved understanding of the relative roles of these diverse NP properties, as well an ability to exert a high level of control over these properties during NP synthesis. While the existing paradigm dictates that decreased size, neutral or negative zeta potential, and polyethylene glycol (PEG) coating (PEGylation) are correlated with increased circulation time (i.e. reduced interaction with host cells),⁴ the manner in which these combined physicochemical properties conspire to direct in vivo cellular interactions has not been elucidated through careful systematic studies, and the nature of these interactions is likely to vary significantly by particle formulation and cell type. As amination of particles is commonly used in various particle modification schemes to enable labeling or targeting, enhance binding and internalization⁵, etc., here we attempt to further elucidate how the exposure of surface amines affects interaction of PEGylated, colloidally stable MSNP with diverse cell types both in vitro and in vivo. In order to directly reveal the influence of amine accessibility on cellular interactions, we synthesized two types of MSNPs with nearly identical size, shape, pore structure, colloidal stability, PEGylation and zeta potential, but differing in exposure and spatial distribution of amines on their surfaces.

It was found that despite the high degree of similarity between the particles (essentially indistinguishable by commonly employed transmission electron microscope (TEM), dynamic light scattering (DLS), and zeta-potential measurements) even very low amounts of exposed surface primary amines dominated the NP-cell interaction with cells in vitro and resulted in rapid clearance from circulation in vivo by interaction with endothelial and white blood cells. Indeed, in vivo, amine accessibility (not zeta potential) was found to alter circulation and vascular binding properties to a significantly greater extent than NP size.

Amorphous MSNPs are widely studied and of broad interest for biomedical applications due to their unique combination of properties including high surface area, tunable pore structure and surface chemistry, high cargo capacity and diversity, and relative lack of toxicity in vivo when administered intravenously.⁶ Additionally, the ability to control size, shape, colloidal stability, and solubility establish MSNPs as an ideal and well-controlled system in which to elucidate the relative role of various particle physicochemical properties on cellular interactions.⁷

To address the relative effect of cationic molecule (amine) exposure on NP interaction with cells, fluorescently labeled MSNPs matched for size, shape, pore structure, PEGylation, and zeta potential (FIG. 2AX and Table 1) were synthesized by modification of a hydrothermal-assisted method described previously⁸ (see SI) using trimethoxy-silylpropyl-modified polyethyleneimine (MW=1500-1800, PEI-silane, its chemical structure was identified by recent work⁹) or N-trimethoxysilylpropyl-N,N,N-trimethyl ammonium chloride (TMAC-silane, MW 258) as the amination reagents (FIG. 1AX) and 2-[methoxy(polyethyleneoxy)-propyl]trimethoxysilane (MW 550-750, 9-12 EO, PEG-silane) for MSNP PEGylation. Compared to alternative colloidal MSNP procedures,¹⁰ this hydrothermal process drives more extensive condensation of silanes, minimizing surface exposure of silanol groups that on their own drive strong NP/cellular interactions.¹¹ By balancing the relative proportions of the branched, higher molecular weight PEI-silane and lower molecular weight TMAC-silane used in the respective synthesis procedures, we were able to prepare size- and charge-matched particles wherein, for PEG-PEI, we expect that the higher molecular weight of the branched PEI to expose primary amines beyond the PEG layer, while for PEG-NMe₃⁺ MSNPs, the quaternary amine of the hydrolyzed TMAC-silane is expected to be more uniformly distributed and partially obstructed within the PEG layer (FIG. 1AX).

TEM imaging and DLS measurements revealed that the basic particle size, shape, pore structure, hydrodynamic size in various solutions, and colloidal stability were nearly identical between PEG-PEI and PEG-NMe₃⁺ MSNPs (FIG. 2AX, Table 1, and FIG. S1). Additionally, zeta potential measurements indicated the particles to be cationic and charge matched (ζ=+40 mV and +20 mV measured in DI water and 10 mM NaCl_{(aq)}, respectively (Table 1 and FIG. S2)). Pore size and total surface area as measured by nitrogen sorption for both particles were ˜2 nm and ˜500 m²/g, respectively (FIG. S3 and Table S1). Measurement of hydrodynamic diameter by DLS in various solutions over time revealed that, while the particles were colloidally stable and nearly identical in diameter when measured in water, PBS, and cell culture medium, an instantaneous ˜20 nm increase in hydrodynamic diameter was observed for only PEG-PEI particles when incubated in medium containing serum (FIG. 2AXc, FIG. S4, and Table 1).

TABLE 1
Physicochemical Properties of PEG-PEI, PEG-NMe₃⁺, and PEG-PEI-ace MS NPs.

| | | | | | | | ζ after |
|---|---|---|---|---|---|---|---|
| | | | | Dₕ in | | | DMEM + |
| | Dₕ in DI | Dₕ in | Dₕ in | DMEM + | ζ in DI | | 10% FBS |
| | water | PBS | DMEM | 10% FBS | water | ζ in 10 mM | incubation |
| Sample | (nm) | (nm) | (nm) | (nm) | (mV) | NaCl_{(aq)}/(mV) | (mV) |
| PEG-PEI | 59.0 ± 1.5 | 59.5 ± 2.3 | 59.5 ± 2.2 | 82.3 ± 3.8 | +40.1 ± 3.0 | +20.3 ± 2.8 | −8.0 ± 3.1 |
| PEG-NMe₃⁺ | 60.1 ± 1.3 | 59.4 ± 1.5 | 59.9 ± 2.0 | 54.4 ± 1.9 | +40.2 ± 2.1 | +20.4 ± 3.5 | +20.6 ± 3.5 |
| PEG-PEI-ace | 59.2 ± 2.4 | 58.4 ± 2.9 | 58.6 ± 1.8 | 54.9 ± 1.8 | +17.8 ± 3.6 | +5.5 ± 3.3 | +7.8 ± 4.3 |

This increase in diameter and surface charge change (+40 to −8 mV, table 1) is attributed to the formation of a protein corona and indicates that, despite their nearly indistinguishable physicochemical parameters routinely measured to assess and predict biomolecular interactions, size-, shape- and charge-matched particles can behave differently in even the most simplified mimic of biological conditions. Given that the combined size and charge of a NP are thought to be predominant factors dictating NP cellular interactions, such as non-specific binding and internalization,⁴ and that cationic NPs in particular show high degrees of non-specific binding and in some cases toxicity,³ we exposed multiple cell types to PEG-PEI and PEG-NMe₃⁺ MSNP. As evident in fluorescence microscopy images in FIG. 3a-c, PEG-PEI particles bind strongly to A549 (human lung carcinoma), A431 (human epithelial cancer), Hep3B (human hepatocellular carcinoma), and human hepatocytes following 30 min exposure (10 μg/mL) under normal cell culture conditions. In contrast, and unexpectedly for a cationic NP, PEG-NMe₃⁺ particles exhibit minimal binding to all cell types under the same conditions (FIG. 3AXb,d and S5). NP binding observed via fluorescence microscopy was confirmed and quantified by flow cytometry (FIG. 3AXd and S5). Although in all cases PEG-PEI MSNPs demonstrated significantly increased binding relative to PEG-NMe₃+ particles, it should be noted that, at the same concentration of NPs, hepatocytes exhibited decreased binding to PEG-PEI particles relative to other cell lines (FIG. S5).

While our primary goal was to elucidate the effect of amine exposure on NP/cellular interactions, this observation that would typically be classified as “non-specific” (i.e. no specific targeting ligand and based on generally ubiquitous charge or chemical interactions) and could be a result of variance in cellular membrane potential as recently described.¹² As the exposed primary amines on the PEG-PEI particles were hypothesized to be responsible for the greatly enhanced binding of otherwise size-, charge-, and PEG-matched particles, primary amines on the PEG-PEI particle were neutralized by titration with acetic anhydride (SI). The acetylated PEG-PEI MSNPs is designated as PEG-PEI-ace. As expected, acetylation of the primary amines reduced the zeta potential, measured to be +20 mV in DI water and +5 mV in 10 mM NaCl_{(aq)}(Table 1). However the particles were shown to be colloidally stable and exhibited no increase in size when introduced into various solutions, including medium with serum (Table 1 and FIG. S4). Furthermore, we observed that elimination of primary amines completely inhibited non-specific particle binding to various cell types (FIG. 3c,d and S5). In all cases, no toxicity was observed with any cell/particle combination after 24 h incubation at various concentrations (12.5-200 μg/mL) as assessed via water-soluble tetrazolium salt (WST-8), lactate dehydrogenase (LDH), (FIG. S6) and hemolysis assays (FIG. S7) conducted with human red blood cells. These results indicate both PEG-PEI and PEG-NMe₃⁺ are highly biocompatible.

While it was anticipated that cationic NPs would bind most cell types,^{2,3,14,15} the relative importance of the exposed amines in otherwise physicochemically matched particles is not generally recognized Indeed, the lack of binding of cationic PEG-NMe₃⁺ (+40 mV) was unanticipated despite the presence of PEG. However, the NP binding studies reported in FIG. 3AX were conducted in vitro without the multitude of competing biological factors found in most in vivo systems. To investigate whether this dramatic differential cellular binding occurs in a complex in vivo system (with blood proteins and cells, endothelial cells, sinusoidal tissue, fenestrated capillaries, a mononuclear phagocyte system, velocity changes, shear forces etc.), we injected 50 μg of 50 nm PEG-PEI, PEG-NMe₃⁺ or PEG-PEI-ace NPs into veins of ex ovo chick embryos as described previously.¹³ This model allows for high resolution, real time in vivo imaging of particle interactions with various cell types and tissues observed within the chorioallantoic membrane (CAM), including white and red blood cells and endothelial cells. A representative video of CAM blood flow and vascular architecture is provided as Video file S1, and representative videos of PEG-PEI and —NMe₃⁺ circulation and binding are provided in Video Files S2 and s3, respectively. Co-injection of PEG-PEI and PEG-NMe₃⁺ particles revealed a dramatic alteration in fate of these size and charge matched particles (FIG. 4AX). PEG-PEI MSNPs (orange) were observed to bind to endothelial cells and stationary and circulating white blood cells, immediately following particle injection, as apparent by the fluorescence intensity on the perimeter of the capillary vessels (endothelial cells) as well more globular/punctate features (white blood cells). In contrast, PEG-NMe₃⁺ MSNPs (FIG. 4AXa-green) remained in circulation for >>6 h post injection (FIG. S8c). Additionally, the location of vascular deposition shifted, with PEG-PEI particles primarily observed in capillaries and bound to white blood cells, while PEG-NMe₃⁺αparticles accumulated very slowly in venules and were taken up more slowly by white blood cells (FIG. S8c). PEG-PEI-ace particles exhibited circulation and accumulation patterns similar to PEG-NMe₃⁺ particles, with minimal immediate or delayed deposition within the capillary bed (FIG. S8d).

This preferential binding of PEG-PEI particles to the capillary endothelium and uptake by white blood cells necessarily reduces the concentration of circulating NPs and diminishes binding to cancer cells in vivo (FIG. 5AX) relative to that observed in vitro (FIG. 3AX). FIG. 5AX shows capillary vascular and white blood cell binding of PEG-PEI with no apparent binding (relative to background) observed to fluorescently labeled A431 cancer cells injected 30 min prior to particle injection. This highlights that in vitro binding—with no competing factors—is not a reliable predictor of in vivo behavior and that PEG-PEI particles bind preferentially to the capillary endothelium and, presumably due to immediate protein corona formation are rapidly scavenged by white blood cells. Similar to the reduction of PEG-PEI particle binding observed on hepatocytes relative to other cell types in vitro, neither PEG-PEI nor PEG-NMe₃⁺ showed non-specific binding to red blood cells as confirmed ex vivo by imaging of isolated red blood cells (FIG. S9). Our findings emphasize the complexity in regulation of even non-specific binding in vivo.

To establish the relative importance of size vs exposed charge on biodistribution and immediate endothelial binding, 150-nm RITC labeled PEG-NMe₃⁺ (TEM images, hydrodynamic size and zeta potential shown in FIG. S10 and Table S2) were synthesized and co-injected with charge-matched 50-nm PEG-PEI particles. Despite the tripling in diameter and ˜27 fold increase in mass, 150-nm PEG-NMe₃⁺ exhibited relatively little immediate binding to the capillary endothelium or scavenging by WBC when compared to 50 nm PEG-PEI particles (FIG. 6AX). These results demonstrate that accessible primary amines play a significant role in regulating NP-cell interactions, overwhelming the role of particle size.

Through synthesis of two types of MSNPs that are matched in terms of primary and hydrodynamic particle size, shape, pore structure, colloidal stability, and zeta potential, our in vitro and in vivo studies have elucidated the relative importance of charged molecule exposure and spatial arrangement vs zeta potential and/or particle size as determinants of non-specific binding and biodistribution. Uniform spatial distribution of charge presented within a PEG background for PEG-NMe₃⁺ confers both colloidal stability and protein corona neutrality (and potentially opsonization neutrality), as evidenced by DLS, which in turn correlate with minimal non-specific binding in vivo and prolonged circulation. Such NP characteristics are expected to be ideal for maximizing the enhanced permeability and retention (EPR) effect or for binding and delivery to targeted circulating cells. In contrast, charge-matched PEG-PEI particles displaying surface-exposed branched amines, although colloidally stable, immediately form a protein corona and exhibit rapid non-specific binding to endothelial and white blood cells and arrest within the CAM. These characteristics are of potential interest for in vivo WBC and vascular labeling. It is also apparent that the combination of size and charge alone are poor predictors of in vivo behavior, and we suggest charge exposure and its effect on protein corona formation and white blood cell scavenging should be additionally considered when designing NPs for in vivo applications.

### 1. Experimental Details

1.1 Chemicals and Reagents.

All chemicals and reagents were used as received. Acetic anhydride, 3-aminopropyltriethoxysilane (98%, APTES), ammonium nitrate (NH₄NO₃), n-cetyltrimethylammonium bromide (CTAB), N,N-dimethyl formamide (DMF), dimethyl sulfoxide (DMSO), rhodamine B isothiocynate (RITC), tetraethyl orthosilicate (TEOS), and trimethylchloromethyl silane (TMS) were purchased from Sigma-Aldrich (St. Louis, Mo.). Ammonium hydroxide (NH₄OH, 28-30%) was obtained from VWR (West Chester, Pa.). Hydrochloric acid (36.5-38%, HCl), and pyridine were purchased from EMD Chemicals (Gibbstown, N.J.). Absolute ethanol was obtained from Pharco-Aaper (Brookfield, Conn.). Trimethoxysilylpropyl modified polyethyleneimine (50% in isopropanol, M.W. 1500-1800, PEI-silane), N-trimethoxysilylpropyl-N,N,N-trimethyl ammonium chloride (50% in methanol, TMAC-silane), and 2-[methoxy(polyethyleneoxy)propyl]trimethoxysilane (Mw 550-750, 9-12 EO, PEG-silane) were purchased from Gelest Inc. (Morrisville, Pa.). Fetal bovine serum (FBS), 10× Phosphate buffered silane (PBS) and penicillin streptomycin (PS) were purchased from Gibco (Logan, Utah). Dulbecco's Modification of Eagle's Medium with 4.5 g/L glucose, L-glutamine and sodium pyruvate (DMEM) was obtained from CORNING cellgro (Manassas, Va.).

1.2 Mesoporous Silica Nanoparticle (MSNP) Synthesis.

Synthesis of stable RITC-incorporated PEG/PEI and PEG/NMe₃⁺ MSNPs (designated as PEG-PEI and PEG-NMe₃⁺): To prepare RITC labeled PEG/PEI MSNPs, 3 mg of RITC was first dissolved in 2 mL of DMF and 1.5 μL of APTES was then added and the stirred at room temperature for 4 h to prepare the RITC-APTES solution. Then, 150 mL of 5.3 mM CTAB ammonium hydroxide solution was prepared in a 250 mL beaker, sealed with parafilm, and placed in an oil bath at 50° C. After continuously stirring for 1 h, 2.5 mL of dilute TEOS solution (prepared in ethanol) was added to the surfactant solution. After another hour stirring, 450 μL of PEG-silane and 20 μL of PEI-silane were added consecutively to the mesoporous silica colloidal solution under stirring. Thirty minutes later, stirring was stopped and the as-synthesized RITC-PEG/PEI colloidal solution was aged at 50° C. for 10 h. After aging, the obtained particle solution (˜50 mL) underwent a hydrothermal treatment reported by Haynes and co-workers.¹ The further NP purification procedure followed a previously reported method.² For RITC labeled PEG-NMe³⁺ MSNP synthesis, the only difference was using TMAC-silane (250 μL) instead of PEI-silane. For large 150 nm PEG-NMe₃⁺ MSNP synthesis, the concentration of NH₄OH and the amount of TMAC-silane were changed 0.512 M of NH₄OH and 300 μL of TMAC-silane, respectively. Otherwise, the synthesis procedure was similar to 50 nm PEG-PEI and PEG-NMe₃⁺ MSNPs.

Acetylation of PEG/PEI MSNPs: For acetylation of PEG/PEI MSNPs, 2 mg of PEG/PEI MSNPs was dispersed to 1 mL of DMF by centrifugation. Then, 20 μL of acetic anhydride and 10 μL of pyridine were added to the PEG/PEI NP solution. After 30 min, the acetylated particles were centrifuged and redispersed in 1 mL of DMF. The acetylation reaction on the particles was repeated one time. The acetylated particles were washed with D.I. water 3 times by centrifugation. Finally, the acetylated PEG-PEI MSNPs were dispersed in DI water at 2 mg/mL.

1.4 Material Characterization.

Nitrogen adsorption-desorption isotherms: The sorption isotherms of RITC labeled PEG-PEI and PEG-NMe³⁺ MSNPs were obtained from on a Micromeritics ASAP 2020 (Norcross, Ga.) at 77 K. Samples were degassed at 120° C. for 12 h before measurements. The surface area and pore size was calculated from the Brunauer-Emmet-Teller (BET) equation and standard Barrett-Joyer-Halenda (BJH) method. Hydrodynamic size and zeta potential measurements: The hydrodynamic size and zeta potential of MSNPs were measured on a Malvern Zetasizer Nano-ZS equipped with a He—Ne laser (633 nm) and Non-Invasive Backscatter optics (NIBS). The samples for dynamic light scattering (DLS) measurements were suspended in various media (DI, PBS, DMEM, and DMEM+10% FBS) at 1 mg/mL. All the hydrodynamic size and zeta potential measurements were carried out at 25° C. Each sample for DLS measurements was obtained at least three runs. The hydrodynamic size of all samples was reported using a z-average diameter. For zeta potential measurements, each sample was at least 100 runs. All the reported values correspond to the average of at least three independent samples.

1.5 Cell Culture.

A-431 (ATCC CRL-1555) human adenocarcinoma, A549 (ATCC CCL-185) human lung carcinoma, Hep3B (ATCC HB-8064) human hepatocellular carcinoma, and human hepatocyte (ATCC CRL-11233) cells from American Type Culture Collection (Manassas, Va.) were maintained in high glucose Dulbecco's Modified Eagle Medium (DMEM) (CORNING Cellgro, Va.) containing 10% fetal bovine serum (FBS) at 37° C. and 5% CO₂.

1.6 Flow Cytometry.

To study the binding difference of PEG-PEI, PEG-NMe₃⁺, and PEG-PEI-ace MSNPs to various types of cells, 5×10⁵ suspension cells (A549, A431, Hep3B, and hepatocyte) were incubated with 10 μg/mL of NPs at 37° C. under 5% CO₂ for 30 min. After NP exposure, cells were washed with PBS twice and then fixed in 1 mL of 3.7% formaldehyde PBS solution for 15 min). After fixation, cells were further washed with PBS one time before flow cytometry measurements. All flow cytometry based measurements were performed in 1×PBS, pH 7.4 buffer (Gibco Life Technologies, Grand Island, N.Y.). Becton-Dickinson FACScalibur flow cytometer (Sunnyvale, Calif.) interfaced to a Power PC Macintosh using the CellQuest software package was used. The instrument is equipped with a 15 mW air-cooled argon ion laser. The laser output is fixed at 488 nm. The signal detection system of the cytometer uses five detectors: a) high performance solid state silicon detector with 488-nm band pass filter for forward scatter detection; b) high performance photomultiplier using Brewster angle beam splitter in the emission optical train for side scatter detection and c) four photomultiplier tubes for fluorescence detection at fixed wavelengths of 530, 585, 670 and 661-nm (FL1, FL2, FL3 and FL4 respectively). Fixed band pass filters are used for wavelength selection and are optimized for the desired emission. For measurements on the flow cytometer, mammalian cell samples were transferred to a tube suitable (Product No. 352008, BD Bioscience, San Jose, Calif.) for flow cytometry and were diluted at least 10-fold in 1×PBS. The dilution step was not only necessary to ensure discrimination between cell-associated fluorescence and background fluorescence arising from unbound NPs, but also ensured that sufficient sample volume for the measurement was used. The required fluorescence signal was detected and recorded on the FL2 channel. Raw data obtained from the flow cytometer were processed using FlowJo software (Tree Star, Inc. Ashland, Oreg.).

1.7 Cell Viability Assay.

Water-soluble tetrazolium salt (WST-8) assay: The cell viability of A431 and A549 after NP incubation was determined using a cell counting kit-8 (CCK-8, Dojindo, Rockville, Md.). First, 6×10⁴ cells were seeded in a 96-well plate and cultured in DMEM+10% FBS at 37° C. under 5% CO₂ for 24 h. Then the cells were incubated with 100 μL of different concentrations of NP solutions. After 24 h incubation, the NP-treated cells were washed with serum-free DMEM two times. After washing, 10 μL of CCK-8 solution was added to each well containing 100 μL of serum-free DMEM. After another 1 h incubation at 37° C. (5% CO₂), the absorbance of the mixture medium was measured at 450 nm using a BioTek microplate reader. The viability was calculated using the following equation.

$$Viability from WST - 8 assay ( \% ) = ( \frac{sample {abs}_{450 nm}}{control {abs}_{450 nm}} ) \times 100$$

Lactate dehydrogenase (LDH) assay: The cell membrane integrity of A549 and A431 cells after NP exposure was assessed by a commonly used LDH assay. The procedure of NP exposure to cells was the same as described in the previous WST-8 assay section. The LDH activity in the cell culture medium was measured using a BioVision LDH cytotoxicity assay kit (Milpitas, Calif.). The percent LDH release from NP treated cells was calculated using the following equation. Cells without NP exposure and lysed cell (by surfactant addition) were used as the negative and positive control, respectively.

$$Percent LDH Release ( \% ) = ( \frac{\begin{matrix}sample {abs}_{455 nm} - \\ negative control\end{matrix}}{\begin{matrix}positive control {abs}_{455 nm} - \\ negative control {abs}_{455 nm}\end{matrix}} ) \times 100$$

1.9 Hemolysis Assay.

Human whole blood (sodium EDTA stabilized) was purchased from Innovative Research (Novi, Mich.). The washed human red blood cells (hRBCs) were obtained by following a published procedure.¹ The washed hRBCs were exposed to PEG-PEI and PEG-NMe₃⁺ MSNPs at different concentrations. All the samples were placed on a rocking shaker in an incubator at 37° C. for 2 h. D.I. water (+hRBCs) and PBS (+hRBCs) was used as the positive control and negative control, respectively. Then the absorbance of hemoglobin in the supernatant was measured by a BioTek microplate reader (Winooski, Vt.) at 541 nm. The hemolysis percentage of each NP treated sample was determined using the following equation.

$$Percent hemolysis ( \% ) = ( \frac{\begin{matrix}sample {abs}_{541 nm} - \\ negative control {abs}_{541 nm}\end{matrix}}{\begin{matrix}positive control {abs}_{541 nm} - \\ negative control {abs}_{541 nm}\end{matrix}} ) \times 100$$

1.8 Ex Ovo Avian Embryos.

The ex ovo chick embryo procedure followed has been described previously³ and was conducted following institutional approval (Protocol 11-100652-T-HSC). Briefly, eggs were acquired from East Mountain Hatchery (Edgewood, N. Mex.) and placed in a GQF 1500 Digital Professional inbubator (Savannah, Ga.) for 3-4 days. Embryos were then removed from shells by cracking into 100 ml polystyrene weigh boats (VWR). Ex ovo embryos were covered and returned to incubator with constant humidity (˜70%) and temperature (˜39° C.). 50 μg (at 1 mg/ml) of NPs in PBS were injected into secondary or tertiary veins via pulled glass capillary needles. Embryo chorioallantoic membrane (CAM) vasculature was imaged using a customized avian embryo chamber (designed by Leong and Lewis) and a Zeiss AxioExaminer upright microscope with heated stage.

TABLE S1
Total surface area and pore size
of PEG-PEI and PEG-NMe₃⁺ MSNPs

| Sample | BET surface area (m²/g) | BJH pore size (nm) |
|---|---|---|
| PEG-PEI | 507 | 2.3 |
| PEG-NMe₃⁺ | 473 | 2.1 |

### Example 4

### Particle Control—Size, Mass, Shape and Surface Chemistry can all be Controlled to Modify Vascular and Single Cell Interaction In Vivo

FIG. 1BX shows monodisperse, colloidally stable MSNPs of the invention having a variety of sizes and charges. FIG. 2BX depicts (1) polydisperse, non-colloidally stable particle shown arresting in vasculature in upper left image (and settled blue pellet in tube in upper right).

(2) (Lower right) Stabilized particles (orange) are observed blowing within the blood vessels and binding to target cancer cells (green). FIG. 2BX shows the highly stable nature of colloidal particles of the invention. The results obtained in this experiment could not be achieved without:
monodispersity; colloidal stability, including in vivo colloidal stability; and control of size, shape, mass (at fixed size) and surface chemistry control.

Size, shape, mass, particle size and surface chemistry can all be modified to alter the type of vessel in which the particles ultimately arrest.

FIG. 3BX shows a comparison of 250 and 50 nm particle separation in blood flow (left) and the almost exclusive deposition of 50 nm particles in venous vasculature 48 hrs post injection (white spots). Next slide demonstrates capillary arrest of 150 nm NPs. Note the deposition of nanoparticles (white dots) almost exclusively in venules. As seen in FIG. 4BX, particle size alters the rate and location of MSNPs deposition. Deposition of 150 nm particles in the capillary bed (right) is depicted at 24 hrs post injection. The type of vasculature in which particles deposit influences ultimate in vivo biodistribution (different tissue and organ types based on vascular structure). Larger particle with same chemistry as previous slide arrested mostly in capillary bed, some in venules and none in arteries.

FIG. 5BX illustrates circulation and organ deposition by particle size. The slides depicted use anionic MSNPs injected intravenously. Not sustained systemic distribution in 50 nm mice. Same observed with 25 nm.

FIGS. 6BX and 7BX also illustrate the relationship of circulation and organ deposition by particle size. The noted pattern continues with: 250 nm particles cleared from bladder and visible primarily in liver, spleen and femur; 150 nm particles similar to 250 nm, with difference primarily in bladder and more in femur; 50 nm particles appear to accumulate more in the liver and bladder, but still distributed systemically; confirmed by ex vivo organ imaging; particles in different states of aggregation (not free dye) visible in the urine.

FIG. 8BX shows untargeted 50 nm MSNP accumulation in a tumor. The particle used here is 50 nm and cationic. Particle described as PEG-PEG-Acetyl in Example 3. Acetylated or quaternary amine modified particles exhibit unusually high tumor uptake (10%+). FIG. 9BX depicts specific targeting with a targeted particle modified from the synthesis described in Example 3. FIG. 10BX depicts single cell binding in vivo and shows an observation of the flow of nanoparticles, as well as interactions with both host and xenograft cells. FIG. 11BX illustrates CD19 mediated targeting of leukemia cells in vivo.

FIG. 12BX shows particle binding specificity in vitro and in vivo (lower right). Particles were synthesized using a method modified from the synthesis described in Example 3. PEG-PEI particles described in Example 3 can also be tuned to binding bacteria in vitro and in vivo, as shown in FIG. 13BX. Binding and surface distribution on bacteria is also modified by size.

### REFERENCES FOR BACKGROUND OF THE INVENTION AND EXAMPLE 3

(1) Nel, A. E.; Madler, L.; Velegol, D.; Xia, T.; Hoek, E. M. V.; Somasundaran, P.; Klaessig, F.; Castranova, V.; Thompson, M. Nat. Mater 2009, 8, 543.
(2) Albanese, A.; Tang, P. S.; Chan. W. C. W. Annu. Rev. Biomed. Eng. 2012, 14, 1.
(3) Dobrovolskaia, M. A.; Aggarwal, P.; Hall, J. B.; McNeil, S. E. Mol. Pharmaceut. 2008, 5, 487.
(4) Wang, J.; Byrne, J. D.; Napier, M. E.; DeSimone, J. M. Small, 2011, 14, 1919.
(5) Rosenholm, J. M.; Meinander, A.; Peuhu, M.; Niemi, R.; Eriksson, J. E.; Sahlgren, C.; Linden, M. ACS Nano. 2009, 3, 197.
(6) Tarn, D.; Ashley, C. E.; Xue, M.; Carnes, E. C.; Zink, J. I.; Brinker, C. J. Acc. Chem. Res. 2013, 46, 792.
(7) Lin, Y.-S.; Hurley, K. R.; Haynes, C. L. J. Phys. Chem. Lett. 2012, 3, 364.
(8) Lin, Y.-S.; Abadeer, N.; Hurley, K. R.; Haynes, C. L. J. Am. Chem. Soc. 2011, 133, 20444.
(9) Dacarro, G; Cucca, L.; Grisoli, P.; Pallavicini, P.; Patrini, M.; Taglietti, A. Dalton Trans. 2012, 41, 2456.
(10) Lai, C.-Y.; Trewyn, B. G; Jeftinija, D. M.; Jeftinija, K.; Xu, S.; Jeftinija, S.; Lin, V. S.-Y. J. Am. Chem. Soc. 2003, 125, 4451.
(11) Zhang, H.; Dunphy, D. R.; Jiang, X.; Meng, H.; Sun, B.; Tarn, D.; Xue, M.; Wang, X.; Lin, S.; Ji, Z.; Li, R.; Garcia, F. L.; Yang, J.; Kirk, M. L.; Xia, T.; Zink, J. I.; Nel, A. E.; Brinker, C. J. J. Am. Chem. Soc. 2012, 134, 15790.
(12) Shin, E. H.; Li, Y.; Kumar, U.; Sureka, H. V.; Zhang, X.; Payne, C. K.; Nanoscale. 2013, 5, 5879.
13) Leong, H. S.; Steinmetz, N. F.; Ablack A.; Destito, G.; Zijlstra, A.; Stuhlmann, H.; Manchester, M; Lewis, J. D. Nat. Protoc. 2010, 5, 1406.
(14) Slowing, I.; Trewyn, B. G; Lin, V. S. Y. J. Am. Chem. Soc. 2006, 128, 14792.
(15) Chung, T. H.; Wu, S. H.; Yao, M.; Lu, C. W.; Lin, Y. S.; Hung, Y; Mou, C. Y.; Chen, Y. C.; Huang D. M. Biomaterials. 2007, 28, 2059.

1. A population of optionally PEGylated, monodisperse mesoporous silica nanoparticles (MSNPs) that are aminated with a composition comprising a primary amine group and that exhibit a non-uniform surface charge distribution and colloidal stability, wherein the MSNPs have a diameter ranging from about 1 nm to about 300 nm, a pore size of between about 1 nm to about 200 nm, a surface area of between about 100-1,000 m²/g, and a Zeta potential (ζ) of between about −40 mV to about +40 mV and wherein upon administration in vivo, the MSNPs exhibit non-specific binding to white blood cells and arterial, venous and/or capillary vessels or combinations thereof, or exhibits minimal non-specific binding and prolonged circulation.

2. The population of MSNPs of claim 1 wherein the MSNPs have a Zeta potential of at least 0 mV.

3. (canceled)

4. The population of MSNPs of any of claims 1-3, wherein the MSNPs comprise one or more components selected from the group consisting of (1) a targeting ligand, and (2) a reporter.

5. (canceled)

6. The population of MSNPs of claim 1, wherein the MSNPs comprise one or more targeting ligands which target white blood cells and/or arterial, venous and/or capillary vessels or targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type ii epithelial cells), and the SP94 peptide (which binds to unknown receptor(s) on hepatocyte-derived cells).

7. The population of MSNPs of claim 1
further comprising a PEGylated lipid bi- or multilayer which encapsulates the population of MSNPs and which comprises (1) at least one lipid and, optionally (2) an optionally-thiolated PEG containing moiety and further optionally (3) at least one targeting ligand which is conjugated to the outer surface of the lipid bi- or multilayer and which is specific against one or more receptors of white blood cells and arterial, venous and/or capillary vessels or combinations thereof.

8. (canceled)

9. The population of claim 7, wherein the lipid bi- or multilayer comprises:
(a) at least one zwitterionic lipid selected from the group consisting of 1, 2-dioleoyl-sn-glycero-3-phosphocholine (DOPC), 1, 2-dioleoyl-sn-glycero-3-phosphoethanolamine (DOPE) and 1,2-di stearoyl-sn-glycero-3-phosphocholine (DSPC); and
(b) optionally, one or more additional electrically charged or neutral lipids selected from the group consisting of 1,2-dipalmitoyl-sn-glycero-3-phosphocholine (DPPC), dioleylglycero triethyleneglycyl iminodiacetic acid (DOIDA), distearylgtycerotdethyleneglycyl iminodiacetic acid (DSIDA), 1,2-dioleoyl-sn-glycero-3-[phosphorserine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

10. The population of claim 7, wherein the targeting ligand is selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type 11 epithelial cells), and the SP94 peptide (which binds to unknown receptor(s) on hepatocyte-derived cells).

11. (canceled)

12. The population of PEGylated MSNPs of claim 1, wherein the MSNPs are aminated with a composition selected from the group consisting of trimethoxysilyl propyl modified polyethyleneimine (MW=1500-1800), (3-aminopropyl)triethoxysilane, (3-aminopropyl)trimethoxy silane, 3-aminopropylmethyl diethoxysilane, 3-aminopropyl dimethoxysilane, or mixtures thereof, or N trimethoxysilylpropyl-N,N,N-trimethyl ammonium chloride (TMAC-silane, MW 258), N¹-(3-Trimethoxylsilylpropyl)diethylenetriamine, 3-(trimethoxysilyl)propyl-di-n-octylmethyl-ammonium chloride; 3-(trimethoxysilyl)propyl-n-octyldimethyl-ammonium chloride; 3-(trimethoxysilyl)propyl-di-n-nonylmethyl-ammonium chloride; 3-(trimethoxysilyl)propyl-n-nonyl dimethyl-ammonium chloride; 3-(trimethoxysilyl)propyl-di-decylmethyl-ammonium chloride; 3-(trimethoxysilyl)propyl-di-n-undecylmethyl-ammonium chloride; 3-(trimethoxysilyl)propyl-n-undecyldimethyl-ammonium chloride; 3-(trimethoxysilyl)propyldi-n-dodecylmethyl-ammonium chloride; 3-(trimethoxysilyl)propyl-n-dodecyldimethyl-ammonium chloride; 3-(trimethoxysilyl)propyl-di-n-tridecyldimethyl-ammonium chloride; 3-(trimethoxysilyl)propyl-n-tridecyldimethyl-ammonium chloride; 3-(trimethoxysilyl)propyl-di-n-tetradecylmethyl-ammonium chloride; 3-(trimethoxysilyl)propyl-n-tetradecyldimethyl-ammonium chloride; 3-(triethoxysilyl)propyl-di-n-octylmethyl-ammonium chloride; 3-(triethoxysilyl)propyl-n-octyldimethyl-ammonium chloride; 3-(triethoxysilyl)propyl-di-n-nonylmethyl-ammonium chloride; 3(triethoxysilyl)propyl-n-nonyldimethyl-ammonium chloride; 3-(triethoxysilyl)propyl-di-n-decylmethyl-ammonium chloride; 3-(triethoxysilyl)propyl-n-decyldimethyl-ammonium chloride; 3-(triethoxysilyl)propyl-di-n-undecylmethyl-ammonium chloride; 3-(triethoxysilyl)propyl-n-undecyldimethyl-ammonium chloride; 3-(triethoxysilyl)propyl-di-n-dodecylmethyl-ammonium chloride; 3-(triethoxysilyl)propyl-n-dodecyldimethyl-ammonium chloride; 3-(triethoxysilyl)propyl-di-n-tridecylmethyl-ammonium chloride; 3-(triethoxysilyl)propyl-n-tridecyldimethyl-ammonium chloride; 3-(triethoxysilyl)propyl-di-n-tetradecylmethyl-ammonium chloride; 3-(triethoxysilyl)propyl-n-tetradecyldimethyl-ammonium chloride; 3-(tripropoxysilyl)propyl-di-n-octylmethyl-ammonium chloride; 3-(tripropoxysilyl)propyl-n-octyldimethyl-ammonium chloride; 3-(tripropoxysilyl)propyl-di-n-nonylmethyl-ammonium chloride; 3-(tripropoxysilyl)propyl-n-nonyldimethyl-ammonium chloride; 3-(tripropoxysilyl)propyl-di-n-decylmethyl-ammonium chloride; 3-(tripropoxysilyl)propyl-n-decyldimethyl-ammonium chloride; 3-(tripropoxysilyl)propyl-di-n-undecylmethyl-ammonium chloride; 3-(tripropoxysilyl)propyl-n-undecyldimethyl-ammonium chloride; 3-(tripropoxysilyl)propyl-di-n-dodecylmethyl-ammonium chloride; 3-(tripropoxysilyl)propyl-n-dodecyldimethyl-ammonium chloride; 3-(tripropoxysilyl)propyl-di-n-tridecylmethyl-ammonium chloride; 3-(tripropoxysilyl)propyl-n-tridecyldimethyl-ammonium chloride; 3-(tripropoxysilyl)propyl-di-n-tetradecylmethyl-ammonium chloride; 3-(tripropoxysilyl)propyl-n-tetradecyldimethyl-ammonium chloride; 3-(tributoxysilyl)propyl-di-n-octylmethyl-ammonium chloride; 3-(tributoxysilyl)propyl-n-octyldimethyl-ammonium chloride; 3-(tributoxysilyl)propyl-di-n-nonylmethyl-ammonium chloride; 3-(tributoxysilyl)propyl-n-nonyldimethyl-ammonium chloride; 3-(tributoxysilyl)propyl-di-n-decylmethyl-ammonium chloride; 3-(tributoxysilyl)propyl-n-decyldimethyl-ammonium chloride; 3-(tributoxysilyl)propyl-di-n-undecylmethyl-ammonium chloride; 3-(tributoxysilyl)propyl-n-undecyldimethyl-ammonium chloride; 3-(tributoxysilyl)propyl-di-n-dodecylmethyl-ammonium chloride; 3-(tributoxysilyl)propyl-n-dodecyldimethyl-ammonium chloride; 3-(tributoxysilyl)propyl-di-n-tridecylmethyl-ammonium chloride; 3-(tributoxysilyl)propyl-n-tridecyldimethyl-ammonium chloride; 3-(tributoxysilyl)propyl-di-n-tetradecylmethyl-ammonium chloride; 3-(tributoxysilyl)propyl-n-tetradecyldimethyl-ammonium chloride and mixtures thereof.

13. (canceled)

14. The population of claim 1 wherein said MSNPs have an average diameter of less than about 30 nm.

15-18. (canceled)

19. The population of claim 1 wherein the MSNP is aminated with a composition selected from the group consisting of trimethoxy-silylpropyl-modified polyethyleneimine (MW=1500-1800, PEI-silane), (3-aminopropyl)triethoxysilane, (3-Aminopropyl)trimethoxysilane, 3-Aminopropylmethyldiethoxysilane, 3-Aminopropyldimethylethoxysilane and mixtures thereof.

20-25. (canceled)

26. A pharmaceutical composition comprising the population of MSNPs of claim 1 loaded with a cargo comprising at least one bioactive agent, in combination with a pharmaceutically acceptable carrier, additive and/or excipient.

27. The pharmaceutical composition of claim 26 wherein the MSNPs have an average diameter ranging from about 1 nm to about 50 nm, loaded with a cargo comprising at least one bioactive agent, in combination with a pharmaceutically acceptable carrier, additive and/or excipient.

28-30. (canceled)

31. The composition according to claim 30 wherein said bioactive agent is at least one vaccine, opiate, hormone, synthetic steroidal agent, fertility agent, contraceptive agent, antibody, or is alfentanil, cyclizine, dexamethasone, diamorphine, glycopyrronium, haloperidol, hydromorphine, hyoscine butylbromide, hyoscine hydrobromide, ketamine, keterolac, levomepromazine, metoclopramide, midazolam, morphine (morphine sulfate), octreotide, oxycodone, a pharmaceutically acceptable salt and/or alternative salt thereof, or a mixture thereof.

32. (canceled)

33. A pharmaceutical composition which is useful in the treatment of one or more disorders selected from the group consisting of a cancer, a bacterial infection, a viral disorder or inflammation of the lungs, kidneys or pancreas and which comprises:
(a) a therapeutically effective amount of a population of PEGylated, monodisperse mesoporous silica nanoparticles (MSNPs) that are aminated with a composition that does not comprise a primary amine group and that exhibit a uniform surface charge distribution and colloidal stability, wherein the MSNPs (1) are loaded with a therapeutically effective amount of one or more active agents that are useful in the treatment of a cancer, a bacterial infection, a viral disorder or inflammation of the lungs, kidneys or pancreas, and (2) have a diameter ranging from about 25 nm to about 300 nm (more preferably, less than 50 nm, even more preferably, less than 30 nm), a pore size of between about 1 nm to about 200 nm, a surface area of between about 100-1,000 m²/g, and a Zeta potential (ζ) of between about −40 mV to about +40 mV (more preferably less than 0 mV) and wherein upon administration in vivo, the MSNPs exhibit minimal non-specific binding and prolonged circulation;
(b) optionally, a reporter; and
(c) optionally, a pharmaceutically acceptable excipient.

34. The composition according to claim 33 wherein said composition that does not comprise a primary amine group is a composition which comprises a quaternary amine group.

35. The composition according to claim 33 wherein said composition that does not comprise a primary amine group is a composition which comprises a secondary and/or a tertiary amine group.

36. (canceled)

37. The composition of claim 33, wherein the MSNPs are encapsulated within a an optionally PEGylated lipid bi- or multilayer which comprises (1) at least one lipid, (2) at least one targeting ligand which is conjugated to the outer surface of the lipid bi- or multilayer and which is specific against one or more receptors of (1) a cancer cell (2) a bacterium (3) a virus, (4) lung tissue, (5) kidney tissue or (6) pancreatic tissue and optionally, (3) at least one PEGylated lipid which is optionally thiolated.

38-39. (canceled)

40. A method of treating cancer, a bacterial infection, a viral disorder, a vascular disorder or inflammation of the lungs, kidneys or pancreas, the method comprising administering to a subject in need thereof the pharmaceutical composition of claim 26.

41. (canceled)

42. The method according to claim 40 wherein said vascular disorder is ischemic stroke, hemorrhagic stroke, transient ischemic attack (TIA), vascular inflammation due to meningitis, atherosclerosis, thrombi or emboli resulting from atherosclerosis, arteritis, physical obstruction of arterial blood supply to the brain, lacunar stroke, hypoperfusion embodying diffuse injury caused by non-localized cerebral ischemia, myocardial infarction and arrhythmia, restenosis associated with percutaneous transluminal coronary angioplasty, peripheral vascular disease and cerebral vascular disease, venous occlusive disorders such as deep vein thrombosis, and hypercoagulopathies, peripheral artery diseases, thrombosis and cerebrovascular disorders, or is aggressive angiomyxoma, anemic infarct, aneurysm, angiopathy, annuloaortic ectasia, aortitis, aortoiliac occlusive disease, arterial stiffness, arteriosclerosis, arteriolosclerosis, atheroma, atherosclerosis, brain ischemia, thomboangitis obliterans, capillaritis, carotid arter dissection, carotid artery stenosis, carotid bruit, cerebral amyloid angiopathy, cholesterol embolism, chronic cerebrospinal venous insufficiency, circulating endothelial cell, collapsed being, corona phlebectatica, degos disease, diabetic angiopathy, diabetic nephropathy, dural arteriovenous fistula, endothelial dysfunction, endotheliitis, familial aortic dissection, fatty streak, fibromuscular dysplasia, hemorrhagi infarct, hereditary cystatin C amyloid angiopathy, Hollenhorst plaque, hot aches, hyaline arteriosclerosis, hyperplastic arteriolosclerosis, hypotension, inferior vena cava syndrome, intermitten claudication, intradural pseudoaneurysm, jugular vein ecstasia, Kawasaki disease, Loeys-Dietz syndrome, macrovascular disease, metanephric dysplatic hematoma of the sacral region, Monckeberg's arteriosclerosis, oblitering endarteritis, orthostatic hypertension, orthostatic hypotension, orthostatic intolerance, Paget-Schroetter disease, paradoxical embolism, peripheral vasculopathy, phlebitis, phlegmasia alba dolens, phlegmasia cerulea dolens, portocaval anastomosis, portal veing thrombosis, postural orthostatic tachycardia syndrome, pseudothrombophlebitis, pulmonary artery sling, pulmonic regurgitation, pylephlebitis, Rasmussen's aneurysm, Sack-Barabas syndrome, Stewart-Treves syndrome, superiod mesenteric artery syndrome, Susac's syndrome, Takyasu's arteritis, thoracic aortic aneurysm, thrombophlebitis, varicocele, vascular anomaly, vascular disease, vascular malformation, vascular malformation, vaso-occlusive crisis, vegetative-vascular dystonia, venous ulcer, vertebral artery dissection and vertobrovasilar insufficiency.

43-53. (canceled)
